SEQUENCE LISTING (98 sequences)

SEQ ID NO: 1 - protein, 371 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNSKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPL

SEQ ID NO: 2 - protein, 371 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATGSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPL

SEQ ID NO: 3 - protein, 173 aa, Homo sapiens
HRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEQRPAVMTSPL

SEQ ID NO: 4 - protein, 371 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPL

SEQ ID NO: 5 - protein, 284 aa, Homo sapiens
MWSWKCLLFWAVLVTATLCTARPSPTLPEQDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPL

SEQ ID NO: 6 - protein, 282 aa, Homo sapiens
MWSWKCLLFWAVLVTATLCTARPSPTLPEQDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPL

SEQ ID NO: 7 - protein, 373 aa, Homo sapiens
MWSWKCLLFWAVLVTATLCTARPCPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYAQILKTAGVNTTDKEMEVLHLRNVSFEDAGEYTCLAGNSIGLSHHSAWLTVLEALEERPAVMTSPL

SEQ ID NO: 8 - protein, 373 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPL

SEQ ID NO: 9 - protein, 62 aa, Homo sapiens
AKTVKFKCPSSGTPNPTLRWLKNSKEFKPDHRIGGYKVRYATWSIIMDSVVPSDKGNYTCIV

SEQ ID NO: 10 - protein, 62 aa, Homo sapiens
AKTVKFKCPSSGTPNPTLRWLKNGKEFKPDHRIGGYKVRYATGSIIMDSVVPSDKGNYTCIV

SEQ ID NO: 11 - protein, 56 aa, Homo sapiens
GDLLQLRCRLRDDVQSINWLRDGVQLAESNRTRITGEEVEVQDSVPADSGLYACVT

SEQ ID NO: 12 - protein, 62 aa, Homo sapiens
AKTVKFKCPSSGTPNPTLRWLKNGKEFKPDHRIGGYKVRYATWSIIMDSVVPSDKGNYTCIV

SEQ ID NO: 13 - protein, 74 aa, Homo sapiens
GSNVEFMCKVYSDPQPHIQWLKHIEVNGSKIGPDNLPYVQILKTAGVNTTDKEMEVLHLRNVSFEDAGEYTCLA

SEQ ID NO: 14 - protein, 74 aa, Homo sapiens
GSNVEFMCKVYSDPQPHIQWLKHIEVNGSKIGPDNLPYAQILKTAGVNTTDKEMEVLHLRNVSFEDAGEYTCLA

SEQ ID NO: 15 - protein, 278 aa, Homo sapiens
MGLTSTWRYGRGPGIGTVTMVSWGRFICLVVVTMATLSLARPSFSLVEDTTLEPEGAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPD

SEQ ID NO: 16 - protein, 262 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVAPGREKEITASPD

SEQ ID NO: 17 - protein, 375 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPD

SEQ ID NO: 18 - protein, 377 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKVLKAAGVNTTDKEIEVLYIRNVTFEDAGEYTCLAGNSIGISFHSAWLTVLPAPGREKEITASPD

SEQ ID NO: 19 - protein, 374 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPD

SEQ ID NO: 20 - protein, 286 aa, Homo sapiens
MVSWGRFICLVVVTMATLSLARPSFSLVEDTTLEPEDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPD

SEQ ID NO: 21 - protein, 285 aa, Homo sapiens
MVSWGRFICLVVVTMATLSLARPSFSLVEDTTLEPEDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPD

SEQ ID NO: 22 - protein, 260 aa, Homo sapiens
MVSWGRFICLVVVTMATLSLARPSFSLVEDTTLEPEGAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPD

SEQ ID NO: 23 - protein, 55 aa, Homo sapiens
GESLEVRCLLKDAAVISWTKDGVHLGPNNRTVLIGEYLQIKGATPRDSGLYACTA

SEQ ID NO: 24 - protein, 62 aa, Homo sapiens
ANTVKFRCPAGGNPMPTMRWLKNGKEFKQEHRIGGYKVRNQHWSLIMESVVPSDKGNYTCVV

SEQ ID NO: 25 - protein, 72 aa, Homo sapiens
GGDVEFVCKVYSDAQPHIQWIKHVEKNGSKYGPDGLPYLKVLKHSGINSSNAEVLALFNVTEADAGEYICKV

SEQ ID NO: 26 - protein, 77 aa, Homo sapiens
GGDVEFVCKVYSDAQPHIQWIKHVEKNGSKYGPDGLPYLKVLKVLKAAGVNTTDKEIEVLYIRNVTFEDAGEYTCLA

SEQ ID NO: 27 - protein, 74 aa, Homo sapiens
GGDVEFVCKVYSDAQPHIQWIKHVEKNGSKYGPDGLPYLKVLKAAGVNTTDKEIEVLYIRNVTFEDAGEYTCLA

SEQ ID NO: 28 - protein, 339 aa, Homo sapiens
VPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGI

SEQ ID NO: 29 - protein, 341 aa, Homo sapiens
VPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKSWISESVEADVRLRLANVSERDGGEYLCRATNFIGVAEKAFWLSVHGPRAAEEELVEADEAGSVYAGI

SEQ ID NO: 30 - protein, 376 aa, Homo sapiens
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGI

SEQ ID NO: 31 - protein, 378 aa, Homo sapiens
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKSWISESVEADVRLRLANVSERDGGEYLCRATNFIGVAEKAFWLSVHGPRAAEEELVEADEAGSVYAGI

SEQ ID NO: 32 - protein, 58 aa, Homo sapiens
GDAVELSCPPPGGGPMGPTVWVKDGTGLVPSERVLVGPQRLQVLNASHEDSGAYSCRQ

SEQ ID NO: 33 - protein, 62 aa, Homo sapiens
ANTVRFRCPAAGNPTPSISWLKNGREFRGEHRIGGIKLRHQQWSLVMESVVPSDRGNYTCVV

SEQ ID NO: 34 - protein, 74 aa, Homo sapiens
GSDVEFHCKVYSDAQPHIQWLKHVEVNGSKVGPDGTPYVTVLKTAGANTTDKELEVLSLHNVTFEDAGEYTCLA

SEQ ID NO: 35 - protein, 73 aa, Homo sapiens
GSDVEFHCKVYSDAQPHIQWLKHVEVNGSKVGPDGTPYVTVLKSWISESVEADVRLRLANVSERDGGEYLCRA

SEQ ID NO: 36 - protein, 369 aa, Homo sapiens
MRLLLALLGILLSVPGPPVLSLEASEEVELEPCLAPSLEQQEQELTVALGQPVRLCCGRAERGGHWYKEGSRLAPAGRVRGWRGRLEIASFLPEDAGRYLCLARGSMIVLQNLTLITGDSLTSSNDDEDPKSHRDLSNRHSYPQQAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLERSPHRPILQAGLPANTTAVVGSDVELLCKVYSDAQPHIQWLKHIVINGSSFGADGFPYVQVLKTADINSSEVEVLYLRNVSAEDAGEYTCLAGNSIGLSYQSAWLTVLPEEDPTWTAAAPEARYTD

SEQ ID NO: 37 - protein, 127 aa, Homo sapiens
QRSPHRPILQAGLPANTTAVVGSDVELLCKVYSDAQPHIQWLKHIVINGSSFGAVGFPYVQVLKTADINSSEVEVLYLRNVSAEDAGEYTCLAGNSIGLSYQSAWLTVLPEEDPTWTAAAPEARYTD

SEQ ID NO: 38 - protein, 369 aa, Homo sapiens
MRLLLALLGVLLSVPGPPVLSLEASEEVELEPCLAPSLEQQEQELTVALGQPVRLCCGRAERGGHWYKEGSRLAPAGRVRGWRGRLEIASFLPEDAGRYLCLARGSMIVLQNLTLITGDSLTSSNDDEDPKSHRDPSNRHSYPQQAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLERSPHRPILQAGLPANTTAVVGSDVELLCKVYSDAQPHIQWLKHIVINGSSFGADGFPYVQVLKTADINSSEVEVLYLRNVSAEDAGEYTCLAGNSIGLSYQSAWLTVLPEEDPTWTAAAPEARYTD

SEQ ID NO: 39 - protein, 369 aa, Homo sapiens
MRLLLALLGVLLSVPGPPVLSLEASEEVELEPCLAPSLEQQEQELTVALGQPVRLCCGRAERGGHWYKEGSRLAPAGRVRGWRGRLEIASFLPEDAGRYLCLARGSMIVLQNLTLITGDSLTSSNDDEDPKSHRDPSNRHSYPQQAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLERSPHRPILQAGLPANTTAVVGSDVELLCKVYSDAQPHIQWLKHIVINGSSFGAVGFPYVQVLKTADINSSEVEVLYLRNVSAEDAGEYTCLAGNSIGLSYQSAWLTVLPEEDPTWTAAAPEARYTD

SEQ ID NO: 40 - protein, 39 aa, Homo sapiens
HWYKEGSRLAPAGRVRGWRGRLEIASFLPEDAGRYLCLA

SEQ ID NO: 41 - protein, 62 aa, Homo sapiens
GNTVKFRCPAAGNPTPTIRWLKDGQAFHGENRIGGIRLRHQHWSLVMESVVPSDRGTYTCLV

SEQ ID NO: 42 - protein, 72 aa, Homo sapiens
GSDVELLCKVYSDAQPHIQWLKHIVINGSSFGADGFPYVQVLKTADINSSEVEVLYLRNVSAEDAGEYTCLA

SEQ ID NO: 43 - protein, 95 aa, Homo sapiens
PTLPEQAQPWGAPVEVESFLVHPGDLLQLRCRLRDDVQSINWLRDGVQLAESNRTRITGEEVEVQDSVPADSGLYACVTSSPSGSDTTYFSVNVS

SEQ ID NO: 44 - protein, 84 aa, Homo sapiens
SPEKMEKKLHAVPAAKTVKFKCPSSGTPNPTLRWLKNGKEFKPDHRIGGYKVRYATWSIIMDSVVPSDKGNYTCIVENEYGSIN

SEQ ID NO: 45 - protein, 103 aa, Homo sapiens
PILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLT

SEQ ID NO: 46 - protein, 95 aa, Homo sapiens
TTLEPEEPPTKYQISQPEVYVAAPGESLEVRCLLKDAAVISWTKDGVHLGPNNRTVLIGEYLQIKGATPRDSGLYACTASRTVDSETWYFMVNVT

SEQ ID NO: 47 - protein, 84 aa, Homo sapiens
NTEKMEKRLHAVPAANTVKFRCPAGGNPMPTMRWLKNGKEFKQEHRIGGYKVRNQHWSLIMESVVPSDKGNYTCVVENEYGSIN

SEQ ID NO: 48 - protein, 101 aa, Homo sapiens
PILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLT

SEQ ID NO: 49 - protein, 97 aa, Homo sapiens
RVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVT

SEQ ID NO: 50 - protein, 84 aa, Homo sapiens
RPERMDKKLLAVPAANTVRFRCPAAGNPTPSISWLKNGREFRGEHRIGGIKLRHQQWSLVMESVVPSDRGNYTCVVENKFGSIR

SEQ ID NO: 51 - protein, 103 aa, Homo sapiens
PILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLV

SEQ ID NO: 52 - protein, 90 aa, Homo sapiens
SEEVELEPCLAPSLEQQEQELTVALGQPVRLCCGRAERGGHWYKEGSRLAPAGRVRGWRGRLEIASFLPEDAGRYLCLARGSMIVLQNLT

SEQ ID NO: 53 - protein, 84 aa, Homo sapiens
HPQRMEKKLHAVPAGNTVKFRCPAAGNPTPTIRWLKDGQAFHGENRIGGIRLRHQHWSLVMESVVPSDRGTYTCLVENAVGSIR

SEQ ID NO: 54 - protein, 101 aa, Homo sapiens
PILQAGLPANTTAVVGSDVELLCKVYSDAQPHIQWLKHIVINGSSFGADGFPYVQVLKTADINSSEVEVLYLRNVSAEDAGEYTCLAGNSIGLSYQSAWLT

SEQ ID NO: 55 - protein, 64 aa, Homo sapiens
MPVAPYWTSPEKMEKKLHAVPAAKTVKFKCPSSGTPNPTLRWLKNGKEFKPDHRIGGYKVRYAT

SEQ ID NO: 56 - protein, 38 aa, Homo sapiens
EYGSINHTYQLDVVERSPHRPILQAGLPANKTVALGSN

SEQ ID NO: 57 - protein, 27 aa, Homo sapiens
KTVALGSNVEFMCKVYSDPQPHIQWLK

SEQ ID NO: 58 - protein, 30 aa, Homo sapiens
PYVQILKTAGVNTTDKEMEVLHLRNVSFED

SEQ ID NO: 59 - protein, 28 aa, Homo sapiens
PYVQILKHSGINSSDAEVLTLFNVTEAQ

SEQ ID NO: 60 - protein, 20 aa, Homo sapiens
TCLAGNSIGLSHHSAWLTVL

SEQ ID NO: 61 - protein, 20 aa, Homo sapiens
VCKVSNYIGEANQSAWLTVT

SEQ ID NO: 62 - protein, 64 aa, Homo sapiens
NKRAPYWTNTEKMEKRLHAVPAANTVKFRCPAGGNPMPTMRWLKNGKEFKQEHRIGGYKVRNQH

SEQ ID NO: 63 - protein, 38 aa, Homo sapiens
EYGSINHTYHLDVVERSPHRPILQAGLPANASTVVGGD

SEQ ID NO: 64 - protein, 27 aa, Homo sapiens
ASTVVGGDVEFVCKVYSDAQPHIQWIK

SEQ ID NO: 65 - protein, 30 aa, Homo sapiens
PYLKVLKAAGVNTTDKEIEVLYIRNVTFED

SEQ ID NO: 66 - protein, 30 aa, Homo sapiens
PYLKVLKHSGINSSNKEAEVLALFNVTEAQ

SEQ ID NO: 67 - protein, 20 aa, Homo sapiens
TCLAGNSIGISFHSAWLTVL

SEQ ID NO: 68 - protein, 20 aa, Homo sapiens
ICKVSNYIGQANQSAWLTVL

SEQ ID NO: 69 - protein, 61 aa, Homo sapiens
APYWTRPERMDKKLLAVPAANTVRFRCPAAGNPTPSISWLKNGREFRGEHRIGGIKLRHQQ

SEQ ID NO: 70 - protein, 38 aa, Homo sapiens
KFGSIRQTYTLDVLERSPHRPILQAGLPANQTAVLGSD

SEQ ID NO: 71 - protein, 27 aa, Homo sapiens
QTAVLGSDVEFHCKVYSDAQPHIQWLK

SEQ ID NO: 72 - protein, 30 aa, Homo sapiens
PYVTVLKTAGANTTDKELEVLSLHNVTFED

SEQ ID NO: 73 - protein, 31 aa, Homo sapiens
PYVTVLKSWISESVEADVRLRLANVSERDGG

SEQ ID NO: 74 - protein, 20 aa, Homo sapiens
TCLAGNSIGFSHHSAWLVVL

SEQ ID NO: 75 - protein, 26 aa, Homo sapiens
RDGGEYLCRATNFIGVAEKAFWLSVH

SEQ ID NO: 76 - protein, 61 aa, Homo sapiens
APYWTHPQRMEKKLHAVPAGNTVKFRCPAAGNPTPTIRWLKDGQAFHGENRIGGIRLRHQH

SEQ ID NO: 77 - protein, 38 aa, Homo sapiens
AVGSIRYNYLLDVLERSPHRPILQAGLPANTTAVVGSD

SEQ ID NO: 78 - protein, 27 aa, Homo sapiens
TTAVVGSDVELLCKVYSDAQPHIQWLK

SEQ ID NO: 79 - protein, 28 aa, Homo sapiens
PYVQVLKTADINSSEVEVLYLRNVSAED

SEQ ID NO: 80 - protein, 20 aa, Homo sapiens
TCLAGNSIGLSYQSAWLTVL

SEQ ID NO: 81 - DNA, 22 bp, Artificial Sequence
  Description of Artificial Sequence Synthetic primer
tgctcaagtcctggatcagtga

SEQ ID NO: 82 - DNA, 20 bp, Artificial Sequence
  Description of Artificial Sequence Synthetic primer
gtgaacgctcagccaaaagg

SEQ ID NO: 83 - DNA, 15 bp, Artificial Sequence
  Description of Artificial Sequence Synthetic probe
tgtgtcggagcggga

SEQ ID NO: 84 - DNA, 25 bp, Artificial Sequence
  Description of Artificial Sequence Synthetic primer
acaaggagctagaggttctctcctt

SEQ ID NO: 85 - DNA, 25 bp, Artificial Sequence
  Description of Artificial Sequence Synthetic primer
gcagagtgatgagaaaacccaatag

SEQ ID NO: 86 - DNA, 17 bp, Artificial Sequence
  Description of Artificial Sequence Synthetic probe
cacctttgaggacgccg

SEQ ID NO: 87 - protein, 19 aa, Homo sapiens
MWSWKCLLFWAVLVTATLC

SEQ ID NO: 88 - protein, 21 aa, Homo sapiens
MWSWKCLLFWAVLVTATLCTA

SEQ ID NO: 89 - protein, 23 aa, Homo sapiens
MWSWKCLLFWAVLVTATLCTARP

SEQ ID NO: 90 - protein, 21 aa, Homo sapiens
MVSWGRFICLVVVTMATLSLA

SEQ ID NO: 91 - protein, 23 aa, Homo sapiens
MVSWGRFICLVVVTMATLSLARP

SEQ ID NO: 92 - protein, 15 aa, Homo sapiens
MGAPACALALCVAVA

SEQ ID NO: 93 - protein, 18 aa, Homo sapiens
MGAPACALALCVAVAIVA

SEQ ID NO: 94 - protein, 20 aa, Homo sapiens
MGAPACALALCVAVAIVAGA

SEQ ID NO: 95 - protein, 22 aa, Homo sapiens
MGAPACALALCVAVAIVAGASS

SEQ ID NO: 96 - protein, 13 aa, Homo sapiens
MRLLLALLGILLS

SEQ ID NO: 97 - protein, 15 aa, Homo sapiens
MRLLLALLGILLSVP

SEQ ID NO: 98 - protein, 16 aa, Homo sapiens
MRLLLALLGILLSVPG

(19) United States
(12) Patent Application Publication
(10) Pub. No.: US 2007/0248605 A1
(43) Pub. Date: Oct. 25, 2007

(54) Fibroblast Growth Factor Receptors 1,2,3, and 4 as Targets for Therapeutic Intervention

(75) Inventors: Kevin Hestir, Kensington, CA (US); Kristen Pierce, Burlingame, CA (US); Lewis Thomas Williams, Burlingame, CA (US); Lorianne Masuoka, Oakland, CA (US); Justin G.P. Wong, Oakland, CA (US); Keting Chu, Woodside, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Five Prime Therapetutics, Inc., South San Francisco, CA (US)

(21) Appl. No.: 10/583,146

(22) PCT Filed: Dec. 17, 2004
(86) PCT No.: PCT/US04/42163

§ 371 Date: Mar. 2, 2007

Related U.S. Application Data
(60) Provisional application No. 60/531,230, filed on Dec. 19, 2003.
(60) Provisional application No. 60/538,349, filed on Jan. 21, 2004.
(60) Provisional application No. 60/616,749, filed on Oct. 6, 2004.

(51) Int. Cl. A61K 39/395; C07K 16/22; G01N 33/53

(52) U.S. Cl. 424/139.1; 435/7.1; 530/387.9

(57) ABSTRACT

A composition is provided that contains a polypeptide and a modulator or a cell comprising the polypeptide and a modulator, where the modulator specifically interferes with the activity of the polypeptide, and the polypeptide is either FGFR3 or FGFR4, inclusive of all polymorphic forms and variants thereof. The modulator can be an antibody or active fragments thereof, a small molecule drug, an RNAi molecule, an antisense molecule or a ribozyme. A method of treatment of tumors in a subject is also provided where an antagonist of FGFR3 or FGFR4 is administered to the subject.

### PRIORITY CLAIM

This application claims the benefit of the following provisional applications filed in the United States Patent and Trademark Office, the disclosures of which are hereby incorporated by reference:

| Application | | |
|---|---|---|
| Number | Title | Filing Date |
| 60/531,230 | Fibroblast Growth Factor Receptors 3 and | Dec. 19, |
| | 4 as Targets for Therapeutic Intervention | 2003 |
| 60/538,349 | Fibroblast Growth Factor Receptors Polypep- | Jan. 21, |
| | tides and Modulators thereof | 2004 |
| Pending | Fibroblast Growth Factor Receptors 1 and | Oct. 6, |
| | 2 as Targets for Therapeutic Intervention | 2004 |

### DESCRIPTION OF THE INVENTION

1. Field of the Invention

The present invention relates to molecules directed to fibroblast growth factor receptors 1, 2, 3, and 4 (“FGFR1, FGFR2, FGFR3, and FGFR4”). Specifically, the invention relates to antibodies directed to FGFR1, FGFR2, FGFR3, and FGFR4 for therapeutic intervention. Additionally, the invention includes methods of treatment, prevention, and diagnosis of diseases, such as proliferative diseases, using the antibodies of the invention.

This application also relates to the field of polypeptides that are over-expressed in cancer, including breast cancer, such as intraductal carcinoma, and lung cancer, such as in adenocarcinomas and/or squamous cell carcinomas, as well as to polynucleotides encoding these polypeptides, extracellular fragments thereof, and antibodies thereto that specifically bind to the polypeptides or specifically modulate the activity of such polypeptides. The invention further relates to diagnostics, cancer vaccines, targets for therapeutic intervention, and methods and compositions for the prophylaxis, treatment, or diagnosis of diseases or conditions, such as proliferative diseases such as cancer, inflammatory diseases, and metabolic disorders.

2. Background of the Invention

Fibroblast growth factors (FGFs) are a family of proteins that interact with heparin sulfate glycosaminoglycans and the extracellular domains of FGF cell surface receptors (FGFRs) to trigger receptor activation and biological responses, as described in Olsen, S. K. et al. (2003), J. Biol. Chem. 278(36): 34226-36 (Epub 2003 June). Other factors, known as FGF homologous factors (FHF1-FHF4, also known as FGF11-FGF14) are related to the FGFs by substantial sequence homology, and by their ability to bind heparin with high affinity, but fail to activate any of the seven principal FGFRs. FGFs are also called heparin binding growth factors (HBGF). Expression of different members of these proteins is found in various tissues and is under particular temporal and spatial control. These proteins are generally potent mitogens for a variety of cell types, such as those of mesodermal, ectodermal, and endodermal origin including, for example, fibroblasts, corneal and vascular endothelial cells, granulocytes, adrenal cortical cells, chondrocytes, myoblasts, vascular smooth muscle cells, lens epithelial cells, melanocytes, keratinocytes, oligodendrocytes, astrocytes, osteoblasts, and hematopoietic cells.

Each member of the FGF family has its unique spectrum of functions as well as functions that overlap with other members of the family or that require interaction with other members of the family. For example, two of the family members, FGF1 and FGF2, have been characterized under many names, but most often as acidic and basic fibroblast growth factor, respectively. The normal gene products influence the general proliferative capacity of the majority of mesodenn and neuroectoderm-derived cells. They are capable of inducing angiogenesis in vivo and may play important roles in early development, as described in Burgess, W. H. and Maciag, T., Ann. Rev. Biochem., 58:575-606 (1989). Further, both FGF1 and FGF2 have the ability to stimulate proliferation and chemotaxis of vascular endothelial cells.

In addition, based on certain studies, both FGF1 and FGF2 have the capacity to stimulate angiogenesis. For example, a eukaryotic expression vector encoding a secreted form of FGF1 has been introduced by gene transfer into porcine arteries. These studies define gene function in the arterial wall in vivo. FGF1 expression induced intimal thickening in porcine arteries 21 days after gene transfer (Nabel, E. G., et al., Nature, 362:844-6 (1993)). They also both have the ability to promote wound healing.

Many other members of the FGF family share similar activities with FGF1 and FGF2, such as promoting angiogellesis and wound healing. Several members of the FGF family have been shown to induce mesoderm formation and to modulate differentiation of neuronal cells, adipocytes, and skeletal muscle cells.

In addition, certain FGFs have been implicated in promoting tumorigenesis in carcinomas and sarcomas by promoting tumor vascularization and as transforming proteins when their expression is deregulated. For example, Pickles, J. O. and Chir, B. (2002), Audiol. Neurootol. 7(1): 36-9, described the activities of FGFs in inner ear development including: the activity of FGF19 in inducing the otocyst followed by the activity of FGF3 in inducing further development of the otocyst; the activities of FGF1 and FGF2, acting as trophic factors for the developing cochlear nerve fibers; and the activities of FGF3 and FGF10 in the development of the walls of the cochlear spaces.

FGF4 has been reported to be active in vitro in maintaining trophoblast stem cells and was found to be required for periimplantation mouse development, as described in Goldin, S. N. and Papaioannou, V. E. (2003), Genesis 36(1): 40-7. Additionally, FGF4 has been found to promote angiogenesis, as described in, for example, Kasahara, H. et al. (2003), J. Am. Coll. Cardiol. 41(6): 1056-62.

Clase, K. L. et al. (2000), Dev. Dyn. 219(3): 368-80 expressed FGF5 ectopically and found that it significantly stimulated proliferation and expansion of tenascin-expressing, connective tissue fibroblast lineage throughout the developing hind limb. The authors suggest that FGF5 acts as a mitogen to stimulate the proliferation of mesenchymal fibroblasts that contribute to the formation of connective tissues and inhibits development of differentiated skeletal muscle.

FGF6 was found to accumulate almost exclusively in the myogenic lineage. Injection of a single dose of recombinant FGF6 was found to upregulate the expression of cyclin D1 mRNA, increase the expression of differentiation markers such as CdkIs, MHCI, and TnI, and accelerate cellular differentiation, as described in Armand, A. S. (2003), Biochim. Biobphys. Acta 1642(1-2): 97-105. FGF7 was found to interact exclusively with one isoform of the FGFR family, FGFR2 IIIb, through interaction between the FGFR2 IIIb unique exon and the beta4/beta5 loop of FGF7, as described in Sher, I. et al. (2003), FEBS Lett. 552(2-3): 150-4. Kinkl, N. et al. (2003), Mol. Cell Neurosci. 23(1): 39-53, examined the effects of FGFR3 and its preferred ligand, FGF9 on survival of adult mammalian retinal ganglion cells (“RGC”) and neurite outgrowth and suggested that the ligand-receptor couple might function to promote survival of adult mammalian RGC.

Hart, A., Papadopoulou, S., and Edlund, H. (2003), Dev. Dyn. 228(2): 185-93, suggested a role for FGF10 and FGFR2b signaling in regulation of pancreatic cell proliferation and differentiation. FGF12 and FGF13 RNAs were detected in the developing central nervous system in mice in cells outside the proliferating ependymal layer. FGF13 RNA was found throughout the peripheral nervous system. FGF12 was found to be expressed in developing soft connective tissue of the limb skeleton of mice. Both genes were found expressed in the myocardium of the heart, with FGF12 RNA found only in the atrial chamber and FGF13 RNA detected in both atrium and ventricle, as described in Hartung, H. et al. (1997), Mech. Dev. 64(1-2): 31-9. Moreover, Leung, K. H. et al. (1998), Biochem. Biophys. Res. Commun. 250(1): 137-42, found that FGF13 induced cell growth of human lung fibroblasts and aortic smooth muscle cells but had no effect on dermal vascular endothelial cells. In contrast, FGF2 induced cell growth in all three cell types.

Many of the above-identified members of the FGF family also bind to the same receptors and elicit a second message through binding to these receptors. Fibroblast growth factors, such as basic FGF, have further been implicated in the growth of Kaposi's sarcoma cells in vitro, Huang, Y. Q., et al., J. Clin. Invest., 91:1191-1197 (1993). Also, the cDNA sequence encoding human basic fibroblast growth factor has been cloned downstream of a transcription promoter recognized by the bacteriophage T7 RNA polymerase. Basic fibroblast growth factors so obtained have been shown to have biological activity indistinguishable from human placental fibroblast growth factor with respect to mitogenicity, synthesis of plasminogen activator, and angiogenesis; Squires, C. H., et. al., J. Biol. Chem., 263:16297 16302 (1988).

U.S. Pat. No. 5,155,214 discloses substantially pure mammalian basic fibroblast growth factors and their production. The amino acid sequences of bovine and human basic fibroblast growth factor are disclosed, as well as the DNA sequence encoding the polypeptide of the bovine species. Newly-discovered FGF9 has approximately 30% sequence similarity to other members of the FGF family. Two cysteine residues and other consensus sequences in family members were also well-conserved in the FGF9 sequence. FGF9 was found to have no typical signal sequence in its N terminus, such as those observed in acidic and basic FGF. However, FGF9 was found to be secreted from cells after synthesis, despite its lack of a typical FGF signal sequence; Miyamoto, M. et al., Mol. and Cell. Biol., 13(7):4251-4259 (1993). Further, FGF9 was found to stimulate the cell growth of oligodendrocyte type 2 astrocyte progenitor cells, BALB/c 3T3, and PC-12 cells, but not growth of human umbilical vein endothelial cells, Naruo, K., et al., J. Biol. Chem., 268:2857-2864 (1993).

Basic FGF and acidic FGF are potent modulators of cell proliferation, cell motility, differentiation, and survival and act on cell types from ectoderm, mesoderm, and endoderm. These two FGFs, along with keratinocyte growth factor (KGF) and androgen induced growth factor (AIGF), were identified by protein purification. However, FGF3, FGF4, FGF5, and FGF6 were isolated as oncogenes, expression of which was restricted to embryogenesis and certain types of cancers. FGF9 was demonstrated to be a mitogen against glial cells. Members of the FGF family are reported to have oncogenic potency. FGF9 has shown transforming potency when transformed into BALB/c 3T3 cells, Miyamoto, M., et. al., Mol. Cell. Biol., 13(7):4251-4259 (1993).

AIGF, also known as FGF8, was purified from a conditioned medium of mouse mammary carcinoma cells (SC-3) simulated with testosterone. AIGF is a distinctive FGF-like growth factor, having a putative signal peptide and sharing 30-40% homology with known members of the FGF family. Mammalian cells transformed with AIGF show a remarkable stimulatory effect on the growth of SC-3 cells in the absence of androgen. Therefore, AIGF mediates androgen-induced growth of SC-3 cells, and perhaps other cells, since it is secreted by the tumor cells themselves; Tanaka, A., et al., Proc. Natl. Acad. Sci. 89(19):8928-3892 (1992).

FGF16 has been identified as a polypeptide containing 207 amino acids, Miyake et al., Biochem. Biophys. Res. Commun., 243(1):148-152 (1998), and appears to have some similarity to FGF9, approximately 73% amino acid identity. The authors found that although the predicted FGF16 amino acid sequence lacked a typical signal sequence, recombinant rat FGF16 was efficiently secreted by Sf9 cells infected with recombinant baculovirus containing cDNA. Additional analysis by Danilenko et al., Arch. Biochem. Biophys., 361(1):34-36 (1999), revealed that the FGF16 protein had a distinct tertiary structure that consisted primarily of beta-strands, had a weak tendency to self-associate, and was fairly extended. Biologic assays showed that d34 rFGF16 induced oligodendrocyte proliferation in vitro, and induced hepatocellular proliferation with increased liver weight in vivo.

In a comparison of the activities of FGF10, FGF16, FGF17, and FGF18 on the human embryonal carcinoma derived cell line Tera-2, it was observed that all four of these FGFs enchanced the survival rate of Tera-2 cells by counteracting apoptosis at concentrations in the interval of approximately 1-10 ng/ml (Engstrom, Anticancer Res., 20(5B):3527-31 (2000)). Higher concentrations of all four of these FGFs exhibited a preferential effect on cell motility was observed.

Fibroblast growth factor receptors (“FGFRs”) bind fibroblast growth factors as ligands and may participate in signaling pathways. At present, over twenty FGFs have been discovered, but only four FGFR genes are known. They are FGFR1-FGFR4. Nevertheless, because of alternative splicing, multiple receptor variants have been found (Johnson, D & Williams, L, Adv. Cancer Res., 60:1 (1993); McKeehan et al., Prog. Nucleic Acid Res. Mol. Biol., 59:135 (1998)). Each receptor appears to have a different ligand-binding capacity and tissue distribution (Orr-Urtreger et al., Dev. Biol., 158:475 (1993); (Peters et al., Dev. Biol., 155:423 (1993); (Partanen et al., Mol. Cell Biol., 12:1698 (1992)). FGFRs have been found to contain an extracellular portion that consists of two or three immunoglobulin-like domains, and a transmembrane element that extends to a cytoplasmic tyrosine kinase. Two extracellular immunoglobulin-like domains (loops 2 and 3) typically comprise the ligand-binding domain. Upon binding of a ligand, FGFR-ligand complexes can dimerize, e.g., in conjunction with a heparan sulphate moiety resulting in tyrosine kinase activation through autophosphorylation (Plotnikov et al., Cell, 98:641 (1999)). These events have been reported to facilitate the binding of second messenger proteins, which in turn can activate various intracellular signaling pathways. It should be noted, however, that additional alternative splicing, that does not alter the FGF-binding domain, generates several other FGFR forms that are assumed to serve some as yet undefined function. For example, it is common to find FGFRs with only the second and third immunoglobulin-like domains, which may or may not extend to the very acidic region (acid box) that lies between immunoglobulin loops 1 and 2.

The C-terminal region of FGFR Ig domain III has been shown to be important for ligand binding and shows specificity toward different ligands. For example, specific mutations in this region in FGFR2 can decrease the binding of FGF2 without affecting the binding of FGF1 or FGF7 (Gray et al., Biochemistry, 34:10325 (1995)). The “b” splice form of FGFR3 (“FGFR3b”) also has unique properties in that it can only be activated by FGF1, which shows little specificity toward any receptor, and FGF9, which shows no activity toward FGFR1b and FGFR2b (Hecht et al., Growth Factors, 12:223 (1995)); (Santos-Ocampo et al., J. Biol. Chem., 271:1726 (1996)).

In PCT publication WO 00/68424, which relates to a means for detecting and treating pathologies linked to FGFR3 and/or to the FGFR3 pathway, FGFR3-IIIb gene mutations in primary tumors are shown (FIGS. 1A-1B), and methods for detecting carcinomas and screening for the FGFR3 mutations are set forth. WO 00/68424 reported several FGFR3 gene mutations in bladder and cervical cancers.

There are also a few reports that, in some breast cancers, FGFR genes are amplified, with amplification of FGFR1 (approximately 20%) and FGFR4 (approximately 30%) observed in a significant number of cases (Theillet et al., Genes Chromosomes Cancer, 7:219 (1993); Adnane et al., Oncogene, 6:659(1991)). In addition, elevated expression of FGFRs was detected using ligand-binding studies with iodinated FGF2 and immunolocalization with an antibody to FGFR1 (Blanckaert et al., Clin. Cancer Res. 4:2939 (1998)).

At present, although there are some intriguing correlations between the expression of FGFs or their receptors in, for example, breast cancer, findings as to the role they play is not fully appreciated. A study by Cappellen et al., Nature Genet., 23:18 (1999), found that a significant proportion of bladder and cervical carcinomas harbor point mutations in FGFR3 that are similar to those that underlie thanatophoric dysplasia, a rare but severe skeletal abnormality of newborn children. Analysis of the mutant receptors has shown that they have acquired ligand independent activity (Neilson, K M & Friesel, R, J. Biol. Chem., 271:25049 (1996); Naski et al., Nature Genet., 13:233 (1996), Webster, M K & Donoghue, D J, EMBO J., 15:520 (1996)). Activating mutations of FGFR1, FGFR2 and FGFR3 have also been found in some craniosynostosis syndromes. Thus, at present, the roles FGFRs play in disease are not fully appreciated. It is desirable to clarify these roles and design methods and compositions that are useful to address FGFR-associated diseases.

### SUMMARY OF THE INVENTION

It is one of the objects of the present invention to provide FGFR polypeptides, polynucleotides encoding such, and agonistic and antagonistic antibodies directed to such. The invention provides the use of such polypeptides, polynucleotides, and antibodies for treatment of diseases, including proliferative diseases, inflammatory diseases, and metabolic disorders.

The antibodies of the invention can be produced by standard techniques known in the art, described herein. These include the culturing and isolation of hybridomas from the spleens of animals immunized with epitopes of FGFR, which secrete antibodies to one or more particular epitopes.

The invention further provides a method for determining the presence of an overexpressed FGFR gene by allowing a polynucleotide or an antibody of the invention to contact a patient sample, and detecting specific binding between the polynucleotide or antibody and any interacting molecule in the sample to determine whether the subject overexpresses the particular gene product. This can be useful for diagnosing a particular disease or disorder, or the propensity to develop a particular disease or disorder, in a subject.

The invention further provides a kit comprising one or more of a polynucleotide, polypeptide, or antibody, which may include instructions for its use. Such kits are useful in therapeutic or diagnostic applications, for example, to detect the presence and/or level of a polypeptide in a biological sample by specific antibody interaction or for treatment of diseases.

### BRIEF DESCRIPTION OF THE TABLES AND DRAWINGS

Table 1 identifies the antibody targets of the invention. Each of the sequences of the invention is identified by an internal reference number (FP ID). Table 1 correlates this reference number with each of the sequences of the invention, as shown in the Sequence Listing. Each sequence is identified by its FP ID number, a SEQ ID NO. corresponding to a polypeptide sequence (SEQ ID NO. (P1)), and a Source ID designation for the source of each antibody target clone and/or fragment thereof. The Source ID combines a protein identification number from publicly-available databases with a designation of the region of the FGFR in which the amino acid sequence is located. Table 1 also designates the FGFR classification as FGFR1, FGFR2, FGFR3, FGFR4. Table 1 further designates the source of the clone as a TM prediction; the Pfam database (described in greater detail below); as an immunoglobin (Ig) domain according to Swiss Prot database or as a contact point between the ligand and receptor; and the region of the FGFR covered by the clone.

Table 2 sets forth the particular FGFR clones that encompass the three Ig domains of each of FGFR1-4: column 1 shows the FP ID; column 2 shows the particular FGFR covered by the clone, i.e., FGFR1-4; column 3 shows the first amino acid coordinate of the relevant Ig domain; column 4 shows the last amino acid coordinate of the relevant Ig domain; and column 5 shows the particular Ig domain covered by the clone, i.e., IgI, IgII, or IgIII.

Table 3 correlates the Source ID of Table 1 with a polynucleotide ID from publicly-available databases. The polypeptide ID correlates with the Source ID of Table 1. Each is further correlated with FGFR1, FGFR2, FGFR3, or FGFR4.

Table 4 sets forth the Pfam coordinates of the polypeptides of the invention: Each is identified by the FP ID, the Source ID, the Pfam designation, ig in the case of each of the clones represented, and the Pfam coordinates for each of the clones.

Table 5 shows the expression of FGFR1 in various malignant tumors found in the GeneLogic proprietary database: column 1 shows the total number of tumors searched; column 2 shows the percentage of those tumors searched that expressed FGFR1; column 3 shows the number of tumors that expressed FGFR1; column 4 shows the site of the tumors; and column 5 shows the particular pathology/morphology of each tumor.

Table 6 shows the expression of FGFR2 in various malignant tumors found in the GeneLogic proprietary database: column 1 shows the total number of tumors searched; column 2 shows the percentage of those tumors searched that expressed FGFR2; column 3 shows the number of tumors that expressed FGFR2; column 4 shows the site of the tumors; and column 5 shows the particular pathology/morphology of each tumor.

Table 7 shows the expression of FGFR3 in various malignant tumors found in the GeneLogic proprietary database: column 1 shows the total number of tumors searched; column 2 shows the percentage of those tumors searched that expressed FGFR3; column 3 shows the number of tumors that expressed FGFR3; column 4 shows the site of the tumors; and column 5 shows the particular pathology/morphology of each tumor.

Table 8 shows the expression of FGFR4 in various malignant tumors found in the GeneLogic proprietary database: column 1 shows the total number of tumors searched; column 2 shows the percentage of those tumors searched that expressed FGFR4; column 3 shows the number to tumors that expressed FGFR4 out of the total searched; column 4 shows the site of the tumors; and column 5 shows the particular pathology/morphology of each tumor.

Table 9 shows the reaction mixtures and thermocycler conditions for the reverse transcription and PCR procedures described in Example 3.

FIG. 1 shows the sequence alignment of each of the clones constituting the extracellular domains of each of FGFR1-4. The left portion of the alignment figure shows the FP ID of each corresponding clone. The middle portion of the figure shows the sequences being aligned. The right portion of the figure shows the codon number where the sequence alignment ends.

FIG. 2 shows the internal control of gene expression used to control for measurements of FGFR3 IIIb and IIIc. Internal 18s rRNA and GAPDH was used to act as internal controls for normalizing gene expression of the FGFR3 isoforms. The values for gene expression were shown numerically in 2(a) and graphically in 2(b). The consistency of the controls for both the 18s rRNA and GAPDH in the each of the three samples depicted demonstrates the reliability of the FGFR3 IIIb and IIIc expression data.

FIG. 3 shows the relative gene expression of FGFR3 IIIb and IIIc in different normal and malignant tissue types. Tissues tested were normal breast, malignant breast, heart, kidney, liver, and lung.

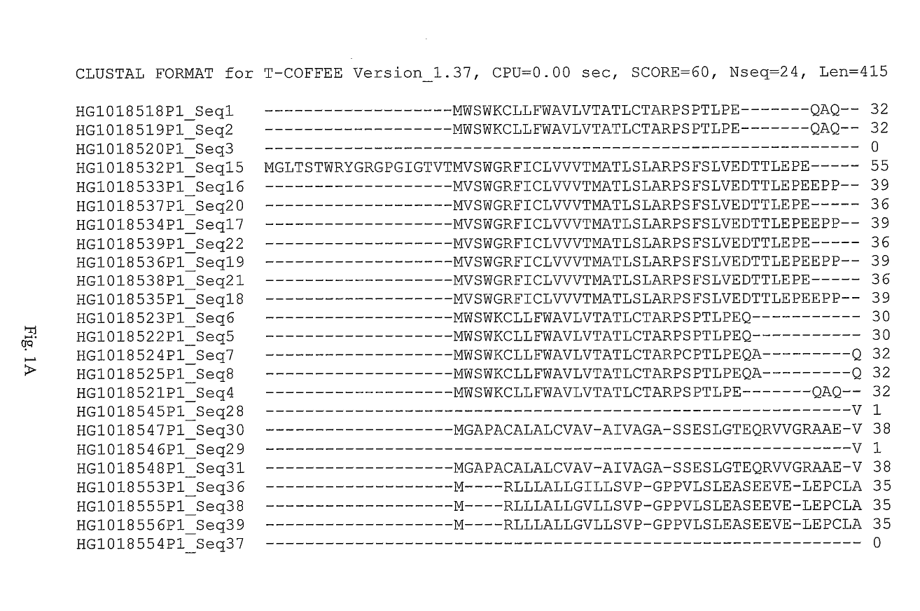

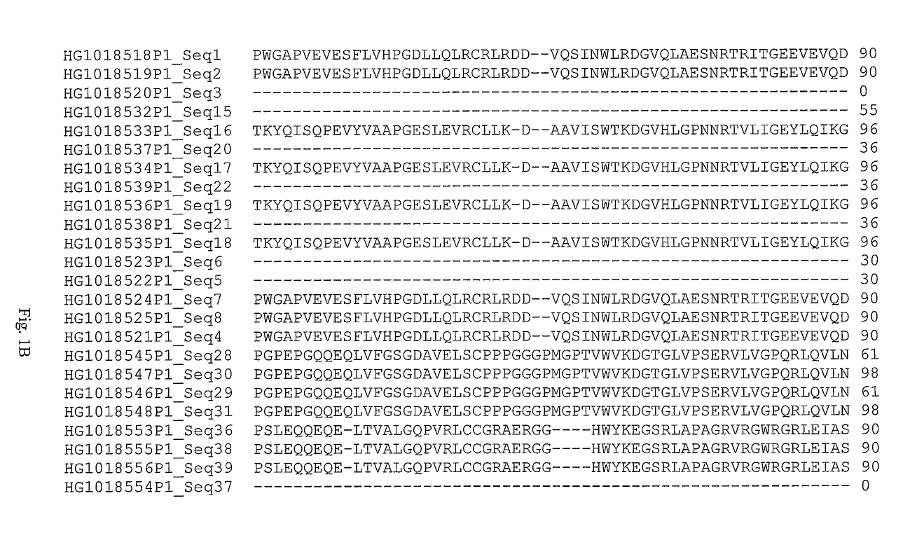

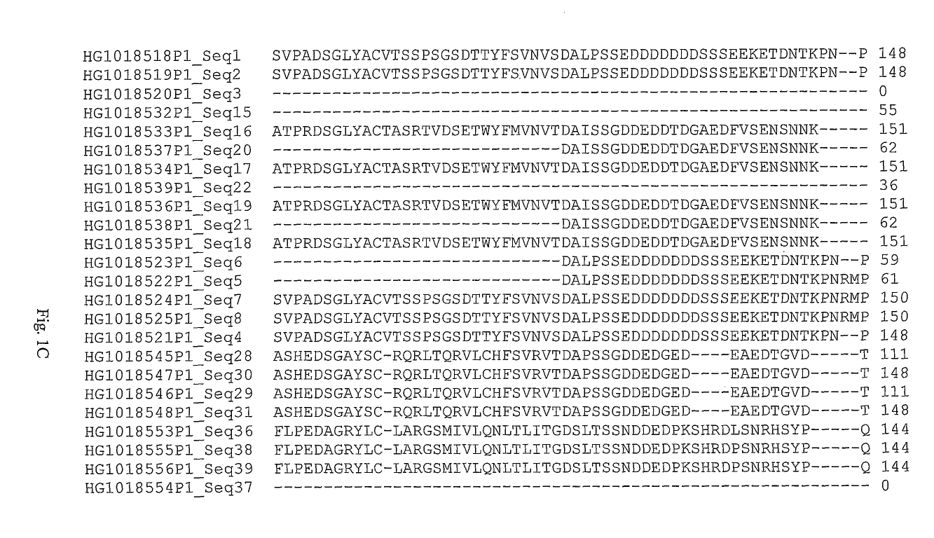

### DETAILED DESCRIPTION OF THE INVENTION

Definitions

“Fibroblast growth factor receptor (FGFR)” refers to any polypeptide that specifically binds one or more fibroblast growth factor. A FGFR typically comprises a transmembrane domain and an extracellular domain with immunoglobulin-like regions. FGFR can include all, any portion or fragment thereof and/or any mutation of such a polypeptide, including soluble fragments of the polypeptide, as well as polymorphic forms and splice variants. “Cell surface FGFR” refers to the extracellular domain, i.e., the portion of the molecule extending outside the cell. Cell surface FGFRs are typically single transmembrane proteins (STM), i.e., they extend into or through the plasma membrane lipid bilayer and span the membrane once. They are numbered herein on the basis of distance from the N-terminus, with the first amino acid residue at the N-terminus as number 1.

An “active fragment” is one having structural, regulatory, or biochemical functions of a naturally occurring molecule or any function related to or associated with a metabolic or physiological process. For example, a fragment demonstrates activity when it participates in a molecular interaction with another molecule, when it has therapeutic value in alleviating a disease condition, or when it has prophylactic value in inducing an immune response to the molecule. Active polypeptide fragments include those exhibiting activity similar, but not necessarily identical, to an activity of a polypeptide set forth herein. The activity may include an improved desired activity, or a decreased undesired activity.

The term “antibody” refers to protein generated by the immune system that is capable of recognizing and binding to a specific antigen. Antibodies, and methods of making antibodies, are commonly known in the art.

An “epitope” is the site of an antigenic molecule to which an antibody binds.

An “agonist antibody” is one that mimics, enhances, stimulates, or activates the function of a molecule with which the agonist interacts.

An “antagonist antibody” is one that competes, inhibits, or interferes with the activity of a molecule with which the antagonist interacts. For example, an antagonist antibody may bind to the receptor without inducing an active response.

The “Fragment antigen binding fragment (Fab fragment) is a disulfide-linked heterodimer, each chain of which contains one immunoglobulin constant region (C) domain and one variable region (V) domain; the juxtaposition of the V domains forms the antigen-binding site. The two Fab fragments of an intact immunoglobulin molecule correspond to its two arms, which typically contain light chain regions paired with the V and C1 domains of the heavy chains.

The “Fragment crystallizable fragment (Fc fragment) is the portion of an antibody molecule that interacts with effector molecules and cells. It comprises the carboxy-terminal portions of the immunoglobulin heavy chains. The functional differences between heavy-chain isotypes lie mainly in the Fc fragment.

The “constant region” of an antibody is its effector region, and determines the functional class of the antibody. The constant region of a heavy or light chain is located at or near the carboxyl terminus.

The “variable region” of an antibody is the region that binds to the antigen; it provides antibody specificity. The variable region of a heavy or light chain is located at or near the amino terminus.

A “polyclonal antibody” a mixture of antibodies of different specificities, as in the serum of an animal immunized to various antigens or epitopes.

A “monoclonal antibody” is an antibody composition having a homogeneous antibody population. The term is not limited with regard to the species or source of the antibody, nor by the manner in which it is made. The term encompasses whole immunoglobulins and immunoglobulin fragments.

A “hybridoma” is a cell hybrid between a lymphocyte and a myeloma cell line.

A “single chain antibody” is an Fab fragment comprising only the V domain of a heavy chain linked by a peptide to a V domain of a light chain.

The “complementarity-determining region (CDR)” is the three dimensional structure of an antibody that provides antigenic specificity.

A “humanized” antibody is an antibody that contains mostly human immunoglobulin sequences. This term is generally used to refer to a non-human immunoglobulin that has been modified to incorporate portions of human sequences, and may include a human antibody that contains entirely human immunoglobulin sequences.

A “primatized” antibody is an antibody that contains mostly primate immunoglobulin sequences. This term is generally used to refer to a non-human immunoglobulin that has been modified to incorporate portions of primate sequences, and may include a primate antibody that contains entirely primate immunoglobulin sequences.

An “isolated,” “purified,” or “substantially isolated” antibody is one that is substantially free of other antibodies and other substances with which it is associated in nature.

A “framework region” is that region of the variable domain that contains relatively invariant sequences and lies between the hypervariable regions. Framework regions provide a protein scaffold for the hypervariable regions.

“Antibody-dependent cell cytotoxicity (ADCC)” is a form of lymphocyte-mediated cytotoxicity in which an effector cell, such as a lymphocyte, mediates the killing of a cell to which an antibody is attached.

The terms “polynucleotide,” “nucleic acid molecule,” and “nucleic acid” are used interchangeably herein to refer to polymeric forms of nucleotides of any length. The nucleic acid molecules can contain deoxyribonucleotides, ribonucleotides, and/or their analogs. Nucleotides can have any three-dimensional structure, and can perform any function, known or unknown. The terms include single-stranded, double-stranded, and triple helical molecules.

A “gene” is an open reading frame encoding a specific protein and/or polypeptide, for example, an mRNA, cDNA, or genomic DNA; it also may or may not include intervening introns, or adjacent 5′ and 3+ non-coding nucleotide sequences involved in the regulation of expression.

A “nucleic acid hybridization reaction” is one in which single strands of DNA or RNA randomly collide with one another, and bind to each other only when their nucleotide sequences have some degree of complementarity. The solvent and temperature conditions can be varied in the reactions to modulate the extent to which the molecules can bind to one another. Hybridization reactions can be performed under different conditions of “stringency.” The “stringency” of a hybridization reaction as used herein refers to the conditions (e.g., solvent and temperature conditions) under which two nucleic acid strands will either pair or fail to pair to form a “hybrid” helix.

The terms “polypeptide” and “protein” refer to a polymer of amino acid residues and are not limited to a minimum length of the product. Thus, peptides, oligopeptides, dimers, and multimers are included within the definition, as are full-length proteins and fragments thereof. The terms also include post-expression modifications of the polypeptide, for example, glycosylation, acetylation, phosphorylation, and the like. Furthermore, for purposes of the present invention, a “polypeptide” refers to a protein which includes modifications, such as deletions, additions, and/or sub substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

A “ligand” is any molecule that binds to a specific site on another molecule.

“Specifically binds,” in the context of antibody binding, refers to high avidity and/or high affinity binding of an antibody to a specific polypeptide, i.e., to an epitope of a polypeptide. Antibody binding to a specific epitope on a polypeptide can be stronger than binding of the same antibody to any other epitopes, particularly other epitopes that can be present in molecules in association with, or in the same sample as the polypeptide of interest. For example, when an antibody binds more strongly to one epitope than to another, adjusting the binding conditions can result in antibody binding almost exclusively to the specific epitope and not to any other epitopes on the same polypeptide, and not to any other polypeptide which does not comprise the epitope. Antibodies that bind specifically to a subject polypeptide may be capable of binding other polypeptides at a weak, yet detectable, level (e.g., 10% or less of the binding shown to the polypeptide of interest). Such weak binding, or background binding, is readily discernible from the specific antibody binding to a subject polypeptide, e.g. by use of appropriate controls. In general, antibodies of the invention bind to a specific polypeptide with a binding affinity of 10-7 M or greater (e.g., 10-8 M, 10-9 M, 10-10, 10-11, etc.).

“Cell proliferation” is an increase in cell number via the growth and reproduction of similar cells.

“Cell repair” means replacing a lost, missing, or defective cellular function, or stimulating an inefficient cellular process.

The terms “subject,” “patient,” and “individual,” used interchangeably herein, refer to a mammal, including, but not limited to, humans, murines, simians, felines, canines, equines, bovines, porcines, ovines, caprines, avians, mammalian farm animals, mammalian sport animals, and mammalian pets.

A “disease” is a pathological, abnormal, and/or hanrful condition of an organism. The term includes conditions, syndromes, and disorders. A “proliferative disease” is a disease or disorder that involves abnormal cell proliferation, including, but not limited to, cancer, psoriasis, and scleroderma.

“Treatment” is the application or administration of remedies or intended remedies for a disease in a subject.

A “pharmaceutically acceptable carrier or excipient” refers to a non-toxic solid, semisolid, or liquid filler, diluent, encapsulating material, or formulation auxiliary of any conventional type. A pharmaceutically acceptable carrier is non-toxic to recipients at the dosages and concentrations employed and is compatible with other ingredients of the formulation.

Antibodies

The present invention provides an antibody that specifically binds to a cell surface FGFR or interferes with binding to a cell surface FGFR. The antibody is either an agonist antibody, which activates the cell surface FGFR, or an antagonist antibody, which interferes with the function of the cell surface FGFR. The cell surface FGFR can be FGFR1, FGFR2, FGFR3, FGFR4, or active fragments of these polypeptides. Furthermore, the antibody can specifically bind to an epitope of FGFR1, FGFR2, FGFR3, or FGFR4, generally in the extracellular domain of the polypeptides. In particular, the antibody will bind to the epitope sequences listed among any of SEQ ID NOs. 1-80. SEQ ID NOs. 1-14, 43-45, and 55-60 are FGFR1 polypeptide sequences. SEQ ID NOs. 15-27, 46-48, and 61-68 are FGFR2 polypeptide sequences. SEQ ID NOs. 28-35, 49-51, and 69-75 are FGFR3 polypeptide sequences. SEQ ID NOs. 36-42, 52-54, and 76-80 are FGFR4 polypeptide sequences.

As noted above, the antibody of the present invention can be an antagonist antibody. Such an antibody may interfere with the binding of other ligands to the receptor. Alternatively, as described above, the antibody can be an agonist antibody. Such an antibody can elicit a functional response from the receptor. The agonist antibody can stimulate cell proliferation or cell repair. The antagonist or agonist antibody can comprise at least one Fab fragment derived from a first antibody that is linked to an F, fragment derived from a second antibody. Furthermore, the first and second antibodies can specifically bind to different epitopes.

Antibodies of the invention can be generated using the entire extracellular domains of any of FGFR1, FGFR2, FGFR3, and/or FGFR4. Animals immunized with the entire extracellular domain can produce polyclonal antibody populations that can be screened for a monoclonal antibody to a selected isotope. Alternatively, animals may be immunized with one or more fragments such as those specified in the Tables and Sequence Listing. The animals herein include mouse, rat, sheep, goat, rabbit, pig, horse, chicken, cow, non-human primate, etc, whether in their native form or “humanized,” as conventional in the art.

Hybrid antibodies of the invention can be developed, as described herein, which do not induce ADCC. For example, as described above, the Fc portion of an IgG1 or IgG2 antibody, which have effector regions that do not induce ADCC, can be combined with Fab regions directed to the amino acid sequences described herein. Antibodies of the invention include all known heavy and light chain isotypes.

Antagonist antibodies of the invention, by inhibiting FGFR function, can inhibit growth of cancer cells. Tumor tissues that overexpress FGFRs are more susceptible to the inhibitory effects of these antibodies than normal cells, which have redundant systems that bypass the growth inhibitory effects of these antibodies. Antagonist antibodies of the invention may also block angiogenesis, thus depriving tumor tissue of oxygen and nutrients.

Agonist antibodies of the invention may stimulate cell growth. They may find use in regenerative medicine and/or treating metabolic diseases. For example, stimulating FGFR function can stimulate or maintain the growth of pancreatic islet cells in the treatment of diabetes, stimulate osteoblasts to treat osteoporosis, stimulate chondrocytes to treat osteoarthritis, and similar uses.

Antibodies of the invention encompasses polyclonal, monoclonal, and single chain antibody preparations, as well as preparations including hybrid antibodies, altered antibodies, chimeric antibodies, and humanized antibodies, as well as hybrid (chimeric) antibody molecules (see, for example, Winter et al., Nature 349:293-299 (1991)); and U.S. Pat. No. 4,816,567); F(ab)₂ and F(ab)₂ fragments; Fv molecules (noncovalent heterodimers, see, for example, Inbar et al., Proc Natl Acad Sci USA 69:2659-2662 (1972)); and Ehrlich et al. (1980) Biochem 19:4091-4096); single-chain Fv molecules (sFv) (see, e.g., Huston et al., Proc Natl Acad Sci USA 85:5879-5883 (1980)); dimeric and trimeric antibody fragment constructs; minibodies (see, e.g., Pack et al., Biochem 31:1579-1584 (1992); Cumber et al., J. Immunology 149B: 120-126 (1992)); humanized antibody molecules (see, e.g., Riechmann et al., Nature 332:323-327 (1988); Verhoeyan et al., Science 239:1534-1536 (1988)); and, any functional fragments obtained from such molecules, wherein such fragments retain specific-binding. Functional fragments of antibodies can include F_{ab} fragments, Fc fragments, cdr fragments, V_{H} fragments, V_{C} fragments, and/or framework fragments.

Antibody molecules of the invention include immunoglobulin molecules, which are typically composed of heavy and light chains, each of which have constant regions that display similarity with other immunoglobulin molecules and variable regions that convey specificity to particular antigens. Most immunoglobulins can be assigned to classes, e.g., IgG, IgM, IgA, IgE, and IgD, based on antigenic determinants in the heavy chain constant region; each class plays a different role in the immune response.

Immunoglobulins are characterized by a structural motif, the imnunoglobulin (ig) domain, which is approximately one hundred amino acids long, is involved in protein-protein and protein-ligand interactions, and includes a conserved intradomain disulfide bond (http://pfam.wustl.edu/cgi-bin/getdesc? name=ig). It is one of the most common domains found among all known proteins, and is present in hundreds of proteins with diverse functions. Proteins with the ig domain comprise the immunoglobulin superfamily; members include antibodies, T-cell receptors, major histocomptability proteins, the CD4, CD8, and CD28 co-receptors, most of the invariant polypeptide chains associated with B and T cell receptors, leukocyte F_{c} receptors, the giant muscle kinase titin, and receptor tyrosine kinases (Janeway et al., 2001; Alberts, et al., 1994).

Antibodies can be used to modulate biological activity, either by increasing or decreasing a stimulation, inhibition, or blockage in the measured activity when compared to a suitable control.

Antibody modulators include antibodies that specifically bind a subject polypeptide and activate the polypeptide, such as receptor-ligand binding that initiates signal transduction; antibodies that specifically bind a subject polypeptide and inhibit binding of another molecule to the polypeptide, thus preventing activation of a signal transduction pathway; antibodies that bind a subject polypeptide to modulate transcription; and antibodies that bind a subject polypeptide to modulate translation. An antibody that modulates a biological activity of a subject polypeptide or polynucleotide increases or decreases the activity or binding at least about 10%, at least about 15%, at least about 20%, at least about 25%, at least about 50%, at least about 100%, or at least about 2-fold, at least about 5-fold, or at least about 10-fold or more when compared to a suitable control. In one embodiment, a modulator of the invention specifically interferes with the activity of a polypeptide, for example, FGFR1, FGFR2, FGFR3, and/or FGFR4. More specifically, the antibody specifically binds to the extracellular domain of FGFR1, FGFR2, FGFR3, or FGFR4.

The antibody can be isolated. In addition, the antibody can bind to or be used to purify any of the polypeptides among the sequences in SEQ ID NOs. 1-80. In some embodiments, the antibody will not be cytotoxic to kidney cells. The antibody can be generated by immunizing an animal with an epitope of the cell surface FGFR and isolating an antibody from a hybridoma derived from a spleen cell that produces an antibody that specifically binds to or interferes with the function of the cell surface FGFR, or mimics, enhances, stimulates, or activates the cell surface FGFR.

The invention provides antibodies that are specific for a subject polypeptide. Suitable antibodies can be produced in a variety of ways conventional in the art, as polyclonal antibodies, monoclonal antibodies, single chain antibodies, and antibody fragments (Harlow et al., 1998; Harlow and Lane, 1988). The antibodies herein include human antibodies, non-human animal antibodies, such as non-human primate antibodies, mouse antibodies, rat antibodies, sheep antibodies, goat antibodies, rabbit antibodies, pig antibodies, cow antibodies, etc., whether in their native form or “humanized,” as conventional in the art. The antibodies herein also include primatized and chimeric antibodies. Further, the present invention includes any such antibodies that are modified to contain a fibronectin backbone or a T-cell receptor backbone.

The antibodies herein can be obtained by immunizing a host animal with polypeptides, or nucleotides encoding polypeptides, comprising all or a portion of the target protein (“immunogen”). Suitable host animals include mouse, rat, sheep, goat, hamster, rabbit, horse, cattle, etc. The host animal may, in certain embodiments, be a different species than the immunogen, e.g., a human protein can be used to immunize mice, etc.

Preferred immunogens comprise all or a part of one of the subject proteins that contain the post-translational modifications, such as glycosylation, found on the native target protein. Immunogens comprising the extracellular domain are produced in a variety of ways known in the art, e.g., expression of cloned genes using conventional recombinant methods, isolation from tumor cell culture supernatants, etc.

Polyclonal antibodies will be provided using conventional techniques, in brief, by in vivo immunization of a host animal with the target protein or immunogen, where the target protein will preferably be in substantially pure form, comprising less than about 1% contaminant. The immunogen may comprise the complete target protein, fragments or derivatives thereof. To increase the immune response of the host animal, the target protein may be combined with an adjuvant, where suitable adjuvants include alum, dextran, sulfate, large polymeric anions, oil & water emulsions, e.g., Freund's adjuvant, Freund's complete adjuvant, and the like. The target protein may also be conjugated to a carrier protein or antigen. A variety of hosts may be immmuized to produce the polyclonal antibodies. Such hosts include rabbits, rodents, e.g., rats, mice, and guinea pigs, sheep, goats, horses, rabbits, chickens, cattle and the like. The target protein is administered to the host, with an initial dosage, with or without the use of adjuvants, followed by one or more, usually at least two, additional booster dosages. Following immunization, the blood from the host will be collected, followed by separation of the serum from the blood cells. The Ig present in the resultant antiserum may be further fractionated using known methods, such as ammonium salt fractionation, DEAE chromatography, and the like.

The method of producing polyclonal antibodies can be varied in some embodiments of the present invention. For example, instead of using a single substantially pure or substantially isolated polypeptide of the present invention as an immunogen, the immunogen composition may contain a number of different immunogens for injection into one animal for simultaneous production of a variety of antibodies to a number of immunogens.

In another embodiment of the present invention, in place of protein immunogens, the immunogens can be nucleic acids that encode the proteins, with or without (such as “naked” DNA) the use of facilitating agents, such as liposomes, microspheres, etc. Such nucleic acids may be in the form of plasmids or vectors.

In a further embodiment of the present invention, polyclonal antibodies can be prepared using phage displayed libraries, conventional in the art. In such a method, a collection of bacteriophages displaying antibody properties on their surfaces are placed in contact with polypeptides of the present invention, whether full length or fragments. Bacteriophages containing antibody properties that specifically recognize the present polypeptides are selected, amplified, for example, in E. coli, and harvested. Such a method typically produces single chain antibodies.

Monoclonal antibodies can also be produced by conventional techniques, such as from hybridomas made from fusing an immortal cell with an antibody producing plasma cell. Generally, the spleen and/or lymph nodes of an immunized host animal provide a source of plasma cells. The plasma cells can be immortalized by fusion with myeloma cells to produce hybridoma cells. Culture supernatant from individual hybridomas can be screened using standard techniques to identify those producing antibodies with the desired specificity. Suitable animals for production of monoclonal antibodies to the human protein include mouse, rat, hamster, etc. To raise antibodies against the mouse protein, the animal will generally be a hamster, guinea pig, rabbit, etc. The antibody may be purified from the hybridoma cell supernatants or ascites fluid by conventional techniques, e.g., affinity chromatography using protein according to the subject invention bound to an insoluble support, protein A sepharose, etc.

The antibody may be produced as a single chain, instead of the normal multimeric stricture (Jost et al., J. Biol. Chem., 269:26267 (1994)). DNA sequences encoding parts of the immunoglobulin, such as for example, the variable region of the heavy chain and the variable region of the light chain or that of two heavy chains or two light chains are ligated to a spacer, such as one encoding at least about 4 amino acids of small neutral amino acids, for example, glycine and/or serine. The protein encoded by this fusion allows assembly of a functional variable region that retains the specificity and affinity of the original antibody.

Other conventional methods of producing antibodies are included in the present invention, such as other methods of producing “artificial” antibodies, e.g., antibodies and antibody fragments produced and selected in vitro. In some embodiments, such antibodies are displayed on the surface of a viral particle. In many embodiments, such artificial antibodies are present as fusion proteins with a viral or bacteriophage structural protein, including, but not limited to, M13 gene III protein. Methods of producing such artificial antibodies are well known in the art. See, e.g., U.S. Pat. Nos. 5,516,637; 5,223,409; 5,658,727; 5,667,988; 5,498,538; 5,403,484; 5,571,698; and 5,625,033.

The present invention includes making antibodies using a library approach. For example, the spleens of immunized animals may be used for extraction of mRNA to isolate the messages that encode antibodies from the immunized animal. Such mRNA may be used to make cDNA libraries. Such a cDNA library may be normalized and subtracted in a manner conventional in the art, for example, to subtract out cDNA hybridizing to mRNA of non-immunized animals. The remaining cDNA may be used to create proteins and for selection of antibody molecules or fragments that specifically bind to the immunogen. The cDNA clones of interest, or fragments thereof, can be introduced into an in vitro expression system that will produce the antibodies. These expression systems can be prokaryotic or eucaryotic. They can be cell-free systems, and can include bacterial, fungal, i.e., yeast, plant, insect, or mammalian cell expression systems. Expression vectors suitable for use in making antibodies include plasmids, retroviruses, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody may be joined to any strong promoter, including retroviral LTRs, e.g., SV-40 early promoter, (Okayama and Berg, 1983), Rous sarcoma virus LTR (Gorman et al., 1982), and moloney murine leukemia virus LTR (Grosschedl and Baltimore, 1985), native Ig promoters, etc.

The present invention includes administration of antibodies into mammals, particularly, humans for therapeutic and/or diagnostic purposes. For in vivo use, particularly for injection into humans, it is desirable to decrease the antigenicity of the antibody. An immune response of a recipient against the antibody will potentially decrease the period of time that the therapy is effective. Thus, antibodies for human use are preferably human antibodies, including those made in animals that have been manipulated to carry human immunoglobulin genes, and those non-human animal antibodies that have been “humanized” by conventional procedures.

Monoclonal antibodies made in non-human animals may be “humanized” prior to administration to humans. Methods of humanizing antibodies are known in the art. The humanized antibody which is the product of an animal having transgenic human immunoglobulin constant region genes is described in, for example, Grosveld and Kolias, 1992; Murphy and Carter, 1993; Pinkert, 1994; WO 90/10077 and WO 90/04036. Alternatively, the antibody of interest may be engineered by recombinant DNA techniques to substitute the CH1, CH2, CH3, hinge domains, and/or the framework domain with the corresponding human sequence, as described in, for example, WO 92/02190.

The present invention also includes chimeric antibodies. The use of Ig cDNA for construction of chimeric imnunoglobulin genes is known in the art (Liu et al., 1987a; Liu et al., 1987b). In this method, mRNA can be isolated from a hybridoma or other cell producing the antibody and is used to produce cDNA. The cDNA of interest may be amplified by the polymerase chain reaction using specific primers, as described in U.S. Pat. Nos. 4,683,195 and 4,683,202. Alternatively, a library can be made and screened to isolate the sequence of interest. The DNA sequence encoding the variable region of the antibody can then be fused to human constant region sequences. The sequences of human constant region genes may be found in Kabat et al., 1991. Human C region genes are readily available from known clones. The choice of isotype will be guided by the desired effector functions, such as the desire to avoid antibody-dependent cellular cytotoxicity. Either of the human light chain constant regions, kappa or lambda, may be used. The chimeric, humanized antibody can then be expressed by conventional methods.

In yet other embodiments, the antibodies may be fully human antibodies. For example, xenogenic antibodies which are identical to human antibodies may be employed. By xenogenic human antibodies is meant antibodies that are the same as human antibodies, i.e., they are fully human antibodies, with the exception that they are produced using a non-human host which has been genetically engineered to express human antibodies, as described in WO 98/50433; WO 98/24893 and WO 99/53049.

Antibody fragments, such as V, F (ab′)₂ and Fab can be prepared by cleaving the intact immunoglobulin, e.g., by protease or chemical cleavage. These fragments can include heavy and light chain variable regions. Alternatively, a truncated gene is designed. For example, a chimeric gene encoding a portion of the F(ab′)₂ fragment might include DNA sequences encoding the CH1 domain and hinge region of the H chain, followed by a translational stop codon to yield the truncated molecule.

Consensus sequences of heavy (H) chain and light (L) chain J regions may be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

Antibodies of the invention can modulate the activity of target cells with which they have primary interactions. They can also modulate the activity of other cells by exerting secondary effects, i.e., when the primary targets interact or communicate with other cells. Antibody modulation of cell activity can be direct or indirect. It includes modulation of transcription, translation, and signal transduction. Antibody modulation of cell activity can inhibit cell growth, and can result in cell death.

Pfam

The FGFRs of the invention encompass a variety of different types of nucleic acids and polypeptides with different structures and functions. They encode or comprise polypeptides belonging to, inter alia, the ig protein family (Pfam). The Pfam system is an organization of protein sequence classification and analysis, based on conserved protein domains; it can be publicly accessed in a number of ways, for example, at http://pfam.wustl.edu. Protein domains are portions of proteins that have a tertiary structure and sometimes have enzymatic or binding activities; multiple domains can be connected by flexible polypeptide regions within a protein. Pfam domains can comprise the N-terminus or the C-terminus of a protein, or can be situated at any point in between. The Pfam system identifies protein families based on these domains and provides an annotated, searchable database that classifies proteins into families (Bateman, A., et al. Nucleic Acids Research 30:276-280 (2000)).

Molecules of the invention can encode or be comprised of one, or more than one, Pfam. Molecules encompassed by the invention include, the polypeptides and polynucleotides shown in the Sequence Listing and corresponding molecular sequences found at all developmental stages of an organism. Molecules of the invention can comprise genes or gene segments designated by the Sequence Listing, and their gene products, i.e., RNA and polypeptides. They also include variants of those set forth in the Sequence Listing that are present in the normal physiological state, e.g., variant alleles such as SNPs and splice variants, as well as variants that are affected in pathological states, such as disease-related mutations or sequences with alterations that lead to pathology, and variants with conservative amino acid changes.

Diagnostic Kits and Methods

The invention provides a kit comprising one or more polypeptides or polypeptide compositions, such as an antibody or antibody composition. The kit may include instructions for its use, which may be provided in a variety of forms, e.g., printed information, compact disc, or other media. Such kits are useful in diagnostic applications, for example, to detect the presence and/or level of a polypeptide in a biological sample by specific antibody interaction. The kit may optionally provide additional useful components, including, but not limited to, buffers, developing reagents, labels, reacting surfaces, means for detections, control samples, standards, and interpretive information.

A kit, or pharmaceutical pack, of the invention can comprise one or more containers filled with one or more of the ingredients of the pharmaceutical compositions of the invention, as described in more detail below. Associated with such container(s) can be a notice in the form prescribed by a governmental agency regulating the manufacture, use, or sale of pharmaceuticals or biological products, which notice reflects approval by the agency of manufacture, use, or sale for human administration.

Kits of the invention for detecting a subject polypeptide will comprise a moiety that specifically binds to a polypeptide of the invention; the moiety includes, but is not limited to, a polypeptide-specific antibody. The kits of the invention can detect one or more molecules of the invention present in biological samples, including biological fluids such as blood, serum, plasma, urine, cerebrospinal fluid, tears, saliva, lymph, dialysis fluid, lavage fluid, semen, and other liquid samples of biological origin. A biological sample can include cells and their progeny, including cells in situ, cells ex vivo, cells in culture, cell supernatants, and cell lysates. It can include organ or tissue culture derived fluids, tissue biopsy samples, tumor biopsy samples, stool samples, and fluids extracted from cells and tissues. Cells dissociated from solid tissues, tissue sections, and cell lysates are also included. A biological sample can comprise a sample that has been manipulated after its procurement, such as by treatment with reagents, solubilization, or enrichment for certain components, such as polynucleotides or polypeptides. Biological samples suitable for use in the kit also include derivatives and fractions of biological samples.

The kits are useful in diagnostic applications. For example, the kit can be used to detect a specific disorder or disease, i.e., a pathological, abnormal, and/or harmful condition which can be identified by symptoms or other identifying factors as diverging from a healthy or a normal state, including syndromes, conditions, and injuries and their resulting damage, e.g., proliferative diseases, such as cancer, inflammatory diseases, and metabolic diseases. Specifically, a kit of the invention can detect some breast cancers and glioblastomas.

The invention provides a method of diagnosing a disease, disorder, syndrome, or condition chosen from cancer, proliferative, inflammatory, immune, metabolic, genetic, disorders, syndromes, or conditions in a patient by providing an antibody that specifically recognizes, binds to, and/or modulates the biological activity of at least one polypeptide according to SEQ ID NOS.: 1-80, or a biologically active fragment or variant thereof, allowing the antibody to contact a patient sample; and detecting specific binding between the antibody and an antigen in the sample to determine whether the subject has such a disease.

The invention also provides a method of diagnosing cancer, proliferative, inflammatory, immune, or metabolic disorder in a patient, by allowing an antibody specific for a polypeptide or a polypeptide of the invention to contact a patient sample, and detecting specific binding between the antibody and any antigen in the sample to determine whether the subject has cancer, proliferative, inflammatory, immune, or metabolic disorder.

The invention provides diagnostic kits and methods for diagnosing disease states based on the detected presence, amount, and/or biological activity of polynucleotides and/or polypeptides in a biological sample. These detection methods can be provided as part of a kit which detects the presence amount, and/or biological activity of a polynucleotide and/or a polypeptide in a biological sample. Procedures using these kits can be performed by clinical laboratories, experimental laboratories, medical practitioners, or private individuals.

Diagnostic methods in which the level of expression is of interest will typically involve determining whether a specific nucleic acid or amino acid molecule is present, and/or comparing its abundance in a sample of interest with that of a control value to determine any relative differences. These differences can then be measured qualitatively and/or quantitatively, and differences related to the presence or absence of an abnormal expression pattern. A variety of different methods for determining the presence or absence of a nucleic acid or polypeptide in a biological sample are known to those of skill in the art; particular methods of interest include those described by Soares, 1997; Pietu et al., 1996; Stolz and Tuan, 1996; Zhao et al., 1995; Chalifour et al., 1994; Raval, 1994; McGraw, 1984; and Hong, 1982. Also of interest are the methods disclosed in WO 97/27317.

Where the kit provides for mRNA detection, detection of hybridization, when compared to a suitable control, is an indication of the presence in the sample of a subject polynucleotide. Appropriate controls include, for example, a sample which is known not to contain subject polynucleotide mRNA, and use of a labeled polynucleotide of the same “sense” as a subject polynucleotide mRNA. Conditions which allow hybridization are known in the art and described in greater detail above. Detection can be accomplished by any known method, including, but not limited to, in situ hybridization, PCR, RT-PCR, and “Northern” or RNA blotting, or combinations of such techniques, using a suitably labeled subject polynucleotide.

Where the kit provides for polypeptide detection, it can include one or more specific antibodies. In some embodiments, the antibody specific to the polypeptide is detectably labeled. In other embodiments, the antibody specific to the polypeptide is not labeled; instead, a second, detectably-labeled antibody is provided that binds to the specific antibody. The kit may further include blocking reagents, buffers, and reagents for developing and/or detecting the detectable marker. The kit may further include instructions for use, controls, and interpretive information.

Detection of specific binding of an antibody, when compared to a suitable control, is an indication that a subject polypeptide is present in the sample. Suitable controls include a sample known not to contain a subject polypeptide; and a sample contacted with an antibody not specific for the subject polypeptide, e.g., an anti-idiotype antibody. A variety of methods to detect specific antibody-antigen interactions are known in the art and can be used in the method, including, but not limited to, standard immunohistological methods, immunoprecipitation, an enzyme immunoassay, and a radioimmunoassay. These methods are known to those skilled in the art (Harlow et al., 1998; Harlow and Lane, 1988).

Where the kit provides for specific antibody detection, it can include one or more polypeptides. In some embodiments, the polypeptide is detectably labeled. In other embodiments, the polypeptide is not labeled; instead, a detectably-labeled ligand or second antibody is provided that specifically binds to the polypeptide. The kit may further include blocking reagents, buffers, and reagents for developing and/or detecting the detectable marker. The kit may further include instructions for use, controls, and interpretive information.

The invention further provides for kits with unit doses of an active agent. These agents are described in more detail below. In some embodiments, the agent is provided in oral or injectable doses. Such kits can comprise a receptacle containing the unit doses and an informational package insert describing the use and attendant benefits of the drugs in treating a condition of interest.

The present invention provides methods for diagnosing disease states based on the detected presence and/or level of polynucleotide or polypeptide in a biological sample, and/or the detected presence and/or level of biological activity of the polynucleotide or polypeptide. These detection methods can be provided as part of a kit. Thus, the invention further provides kits for detecting the presence and/or a level of a polynucleotide or polypeptide in a biological sample and/or or the detected presence and/or level of biological activity of the polynucleotide or polypeptide. Procedures using these kits can be performed by clinical laboratories, experimental laboratories, medical practitioners, or private individuals.

Method of Treatment

The present invention provides a method for treating diseases including proliferative diseases, inflammatory diseases, and metabolic disorders. The method of the invention provides for treating these diseases with antibodies. It also provides for treating these diseases when they have proven refractory to other treatments. For example, the methods of the invention are useful in treating diseases that have proven refractory to treatment with other antibodies. In an embodiment, the invention can be used to treat diseases refractory to treatment with anti-HER2 antibodies or anti-EGFR antibodies. This method includes administering antibodies to epitopes of FGFR1, FGFR2, FGFR3, and/or FGFR4 to a subject. The method of treatment can be for a proliferative disease and in particular, cancer. Cancers that can be treated with antibodies of the invention include melanoma, glioblastoma, carcinoma, breast, pancreatic, ovarian, prostate, bladder, rectal, colon, lung, and stomach. Inflammatory diseases include rheumatoid arthritis, osteoarthritis, psoriasis, inflammatory bowel disease, multiple sclerosis, SLE, myocardial infarction, stroke, and fulminant liver failure. Metabolic disorders include type II diabetes, phosphatemia, and osteoporosis.

The antibody is administered locally or systemically. In addition, the antibody is administered intravenously, intra-peritoneally, sub-cutaneously, topically, or transdermally. Furthermore, the antibody is used in a composition with a pharmaceutically acceptable carrier or excipient. A “pharmaceutically acceptable carrier” or “excipient” is intended to include substances that can be co-administered with the compositions of the invention that allows the composition or active molecule therein to perform its intended function. Examples of such carriers include solutions, solvents, buffers, dispersion media, delay agents, emulsions and the like. Further, any other conventional carrier suitable for use with the described antibodies fall within the scope of the instant invention, such as, for example, phosphate buffered saline. The treatment includes administering a therapeutically effective amount of the antibody composition to the subject.

Method of Detecting FGFR1-4 Gene Products

The invention provides for a method of detecting the presence of an amplified gene encoding cell surface FGFRs in a subject. The method comprises detection of hybridization between a polynucleotide probe and a nucleic acid molecule encoding the cell surface polypeptide obtained from a subject under stringent hybridization conditions. The polynucleotide probe can be chosen from any of the sequences that encode SEQ ID NOs. 1-80.

TABLE 1
Identification of FGFR Antibody Targets

| | | | | | Region |
|---|---|---|---|---|---|
| FP ID | SEQ. ID. NO. (P1) | Source ID | Type | Source type | Type |
| HG1018518 | SEQ. ID. NO. 1 | 182530_ECD | FGFR1 | TM prediction | ECD |
| HG1018519 | SEQ. ID. NO. 2 | 22450878_ECD | FGFR1 | TM prediction | ECD |
| HG1018520 | SEQ. ID. NO. 3 | 558584_ECD | FGFR1 | TM prediction | ECD |
| HG1018521 | SEQ. ID. NO. 4 | NP_056934_ECD | FGFR1 | TM prediction | ECD |
| HG1018522 | SEQ. ID. NO. 5 | NP_075593_ECD | FGFR1 | TM prediction | ECD |
| HG1018523 | SEQ. ID. NO. 6 | NP_075594_ECD | FGFR1 | TM prediction | ECD |
| HG1018524 | SEQ. ID. NO. 7 | NP_075597_ECD | FGFR1 | TM prediction | ECD |
| HG1018525 | SEQ. ID. NO. 8 | NP_075599_ECD | FGFR1 | TM prediction | ECD |
| HG1018526 | SEQ. ID. NO. 9 | 182530_ig2 | FGFR1 | pfam | IgII |
| HG1018527 | SEQ. ID. NO. 10 | 22450878_ig2 | FGFR1 | pfam | IgII |
| HG1018528 | SEQ. ID. NO. 11 | NP_056934_ig1 | FGFR1 | pfam | IgI |
| HG1018529 | SEQ. ID. NO. 12 | NP_056934_ig2 | FGFR1 | pfam | IgII |
| HG1018530 | SEQ. ID. NO. 13 | NP_056934_ig3 | FGFR1 | pfam | IgIII |
| HG1018531 | SEQ. ID. NO. 14 | NP_075597_ig3 | FGFR1 | pfam | IgIII |
| HG1018532 | SEQ. ID. NO. 15 | 25058745_ECD | FGFR2 | TM prediction | ECD |
| HG1018533 | SEQ. ID. NO. 16 | 27260913_ECD | FGFR2 | TM prediction | ECD |
| HG1018534 | SEQ. ID. NO. 17 | NP_075258_ECD | FGFR2 | TM prediction | ECD |
| HG1018535 | SEQ. ID. NO. 18 | NP_075261_ECD | FGFR2 | TM prediction | ECD |
| HG1018536 | SEQ. ID. NO. 19 | NP_075262_ECD | FGFR2 | TM prediction | ECD |
| HG1018537 | SEQ. ID. NO. 20 | NP_075264_ECD | FGFR2 | TM prediction | ECD |
| HG1018538 | SEQ. ID. NO. 21 | NP_075418_ECD | FGFR2 | TM prediction | ECD |
| HG1018539 | SEQ. ID. NO. 22 | NP_075419_ECD | FGFR2 | TM prediction | ECD |
| HG1018540 | SEQ. ID. NO. 23 | NP_075258_ig1 | FGFR2 | pfam | IgI |
| HG1018541 | SEQ. ID. NO. 24 | NP_075258_ig2 | FGFR2 | pfam | IgII |
| HG1018542 | SEQ. ID. NO. 25 | NP_075258_ig3 | FGFR2 | pfam | IgIII |
| HG1018543 | SEQ. ID. NO. 26 | NP_075261_ig3 | FGFR2 | pfam | IgIII |
| HG1018544 | SEQ. ID. NO. 27 | NP_075262_ig3 | FGFR2 | pfam | IgIII |
| HG1018545 | SEQ. ID. NO. 28 | 20452380_ECD | FGFR3 | TM prediction | ECD |
| HG1018546 | SEQ. ID. NO. 29 | 20452381_ECD | FGFR3 | TM prediction | ECD |
| HG1018547 | SEQ. ID. NO. 30 | 4503711_ECD | FGFR3 | TM prediction | ECD |
| HG1018548 | SEQ. ID. NO. 31 | NovelFGFR3_clone021_ECD | FGFR3 | TM prediction | ECD |
| HG1018549 | SEQ. ID. NO. 32 | 4503711_ig1 | FGFR3 | pfam | IgI |
| HG1018550 | SEQ. ID. NO. 33 | 4503711_ig2 | FGFR3 | pfam | IgII |
| HG1018551 | SEQ. ID. NO. 34 | 4503711_ig3 | FGFR3 | pfam | IgIII |
| HG1018552 | SEQ. ID. NO. 35 | 20452381_ig3 | FGFR3 | pfam | IgIII |
| HG1018553 | SEQ. ID. NO. 36 | 2832350_ECD | FGFR4 | TM prediction | ECD |
| HG1018554 | SEQ. ID. NO. 37 | 7018380_ECD | FGFR4 | TM prediction | ECD |
| HG1018555 | SEQ. ID. NO. 38 | NP_002002_ECD | FGFR4 | TM prediction | ECD |
| HG1018556 | SEQ. ID. NO. 39 | proteinkinase113A_ECD | FGFR4 | TM prediction | ECD |
| HG1018557 | SEQ. ID. NO. 40 | NP_002002_ig1 | FGFR4 | pfam | IgI |
| HG1018558 | SEQ. ID. NO. 41 | NP_002002_ig2 | FGFR4 | pfam | IgII |
| HG1018559 | SEQ. ID. NO. 42 | NP_002002_ig3 | FGFR4 | pfam | IgIII |
| HG1018560 | SEQ. ID. NO. 43 | FGFR1_ig1 | FGFR1 | Ig domain | IgI |
| HG1018561 | SEQ. ID. NO. 44 | FGFR1_ig2 | FGFR1 | Ig domain | IgII |
| HG1018562 | SEQ. ID. NO. 45 | FGFR1_ig3 | FGFR1 | Ig domain | IgIII |
| HG1018563 | SEQ. ID. NO. 46 | FGFR2_ig1 | FGFR2 | Ig domain | IgI |
| HG1018564 | SEQ. ID. NO. 47 | FGFR2_ig2 | FGFR2 | Ig domain | IgII |
| HG1018565 | SEQ. ID. NO. 48 | FGFR2_ig3 | FGFR2 | Ig domain | IgIII |
| HG1018566 | SEQ. ID. NO. 49 | FGFR3_ig1 | FGFR3 | Ig domain | IgI |
| HG1018567 | SEQ. ID. NO. 50 | FGFR3_ig2 | FGFR3 | Ig domain | IgII |
| HG1018568 | SEQ. ID. NO. 51 | FGFR3_ig3 | FGFR3 | Ig domain | IgIII |
| HG1018569 | SEQ. ID. NO. 52 | FGFR4_ig1 | FGFR4 | Ig domain | IgI |
| HG1018570 | SEQ. ID. NO. 53 | FGFR4_ig2 | FGFR4 | Ig domain | IgII |
| HG1018571 | SEQ. ID. NO. 54 | FGFR4_ig3 | FGFR4 | Ig domain | IgIII |
| HG1018572 | SEQ. ID. NO. 55 | FGFR1_contactregion_Seq1 | FGFR1 | ContactPoint | in-between |
| HG1018573 | SEQ. ID. NO. 56 | FGFR1_contactregion_Seq2 | FGFR1 | ContactPoint | in-between |
| HG1018574 | SEQ. ID. NO. 57 | FGFR1_contactregion_Seq3 | FGFR1 | ContactPoint | in-between |
| HG1018575 | SEQ. ID. NO. 58 | FGFR1_contactregion_Seq4 | FGFR1 | ContactPoint | in-between |
| HG1018576 | SEQ. ID. NO. 59 | FGFR1_contactregion_Seq5 | FGFR1 | ContactPoint | in-between |
| HG1018577 | SEQ. ID. NO. 60 | FGFR1_contactregion_Seq6 | FGFR1 | ContactPoint | in-between |
| HG1018578 | SEQ. ID. NO. 61 | FGFR1_contactregion_Seq7 | FGFR2 | ContactPoint | in-between |
| HG1018579 | SEQ. ID. NO. 62 | FGFR2_contactregion_Seq1 | FGFR2 | ContactPoint | in-between |
| HG1018580 | SEQ. ID. NO. 63 | FGFR2_contactregion_Seq2 | FGFR2 | ContactPoint | in-between |
| HG1018581 | SEQ. ID. NO. 64 | FGFR2_contactregion_Seq3 | FGFR2 | ContactPoint | in-between |
| HG1018582 | SEQ. ID. NO. 65 | FGFR2_contactregion_Seq4 | FGFR2 | ContactPoint | in-between |
| HG1018583 | SEQ. ID. NO. 66 | FGFR2_contactregion_Seq5 | FGFR2 | ContactPoint | in-between |
| HG1018584 | SEQ. ID. NO. 67 | FGFR2_contactregion_Seq6 | FGFR2 | ContactPoint | in-between |
| HG1018585 | SEQ. ID. NO. 68 | FGFR2_contactregion_Seq7 | FGFR2 | ContactPoint | in-between |
| HG1018586 | SEQ. ID. NO. 69 | FGFR3_contactregion_Seq1 | FGFR3 | ContactPoint | in-between |
| HG1018587 | SEQ. ID. NO. 70 | FGFR3_contactregion_Seq2 | FGFR3 | ContactPoint | in-between |
| HG1018588 | SEQ. ID. NO. 71 | FGFR3_contactregion_Seq3 | FGFR3 | ContactPoint | in-between |
| HG1018589 | SEQ. ID. NO. 72 | FGFR3_contactregion_Seq4 | FGFR3 | ContactPoint | in-between |
| HG1018590 | SEQ. ID. NO. 73 | FGFR3_contactregion_Seq5 | FGFR3 | ContactPoint | in-between |
| HG1018591 | SEQ. ID. NO. 74 | FGFR3_contactregion_Seq6 | FGFR3 | ContactPoint | in-between |
| HG1018592 | SEQ. ID. NO. 75 | FGFR3_contactregion_Seq7 | FGFR3 | ContactPoint | in-between |
| HG1018593 | SEQ. ID. NO. 76 | FGFR4_contactregion_Seq1 | FGFR4 | ContactPoint | in-between |
| HG1018594 | SEQ. ID. NO. 77 | FGFR4_contactregion_Seq2 | FGFR4 | ContactPoint | in-between |
| HG1018595 | SEQ. ID. NO. 78 | FGFR4_contactregion_Seq3 | FGFR4 | ContactPoint | in-between |
| HG1018596 | SEQ. ID. NO. 79 | FGFR4_contactregion_Seq4 | FGFR4 | ContactPoint | in-between |
| HG1018597 | SEQ. ID. NO. 80 | FGFR4_contactregion_Seq5 | FGFR4 | ContactPoint | in-between |

TABLE 2
Immunoglobulin Domain Coordinates

| FP ID | FGFR | Ig Start | Ig Stop | Type |
|---|---|---|---|---|
| HG1018560 | FGFR1 | 25 | 119 | IgI |
| HG1018561 | FGFR1 | 155 | 238 | IgII |
| HG1018562 | FGFR1 | 253 | 355 | IgIII |
| HG1018563 | FGFR2 | 31 | 125 | IgI |
| HG1018564 | FGFR2 | 158 | 241 | IgII |
| HG1018565 | FGFR2 | 256 | 356 | IgIII |
| HG1018566 | FGFR3 | 30 | 126 | IgI |
| HG1018567 | FGFR3 | 155 | 238 | IgII |
| HG1018568 | FGFR3 | 253 | 355 | IgIII |
| HG1018569 | FGFR4 | 25 | 114 | IgI |
| HG1018570 | FGFR4 | 151 | 234 | IgII |
| HG1018571 | FGFR4 | 249 | 349 | IgIII |

TABLE 3
Correlation of Source ID with Nucleotide ID

| Polypeptide ID | Polynucleotide ID | FGFR |
|---|---|---|
| 182530 | 182529 | FGFR1 |
| 22450878 | 22450877 | FGFR1 |
| 558584 | 558583 | FGFR1 |
| NP_056934 | NM_015850 | FGFR1 |
| NP_075593 | NM_023105 | FGFR1 |
| NP_075594 | NM_023106 | FGFR1 |
| NP_075597 | NM_023109 | FGFR1 |
| NP_075599 | NM_023111 | FGFR1 |
| 25058745 | 25058744 | FGFR2 |
| 27260913 | 27260912 | FGFR2 |
| NP_075258 | NM_022969 | FGFR2 |
| NP_075261 | NM_022972 | FGFR2 |
| NP_075262 | NM_022973 | FGFR2 |
| NP_075264 | NM_022975 | FGFR2 |
| NP_075418 | NM_023029 | FGFR2 |
| NP_075419 | NM_023030 | FGFR2 |
| 20452380 | 13112046 | FGFR3 |
| 20452381 | 13112046 | FGFR3 |
| 4503711 | 13112046 | FGFR3 |
| 2832350 | 13112051 | FGFR4 |
| 7018380 | 31371 | FGFR4 |
| NP_002002 | NM_002011 | FGFR4 |
| proteinkinase113A | proteinkinase113B | FGFR4 |

TABLE 4
Pfam Coordinates

| FP Patent ID | Source ID | Pfam | Coordinates |
|---|---|---|---|
| HG1018518 | 182530_ECD | ig | (268-341) |
| HG1018518 | 182530_ECD | ig | (48-103) |
| HG1018518 | 182530_ECD | ig | (169-230) |
| HG1018519 | 22450878_ECD | ig | (268-341) |
| HG1018519 | 22450878_ECD | ig | (169-230) |
| HG1018519 | 22450878_ECD | ig | (48-103) |
| HG1018520 | 558584_ECD | ig | (70-143) |
| HG1018520 | 558584_ECD | ig | (14-32) |
| HG1018521 | NP_056934_ECD | ig | (268-341) |
| HG1018521 | NP_056934_ECD | ig | (169-230) |
| HG1018521 | NP_056934_ECD | ig | (48-103) |
| HG1018522 | NP_075593_ECD | ig | (181-254) |
| HG1018522 | NP_075593_ECD | ig | (82-143) |
| HG1018523 | NP_075594_ECD | ig | (179-252) |
| HG1018523 | NP_075594_ECD | ig | (80-141) |
| HG1018524 | NP_075597_ECD | ig | (270-343) |
| HG1018524 | NP_075597_ECD | ig | (171-232) |
| HG1018524 | NP_075597_ECD | ig | (48-103) |
| HG1018525 | NP_075599_ECD | ig | (270-343) |
| HG1018525 | NP_075599_ECD | ig | (171-232) |
| HG1018525 | NP_075599_ECD | ig | (48-103) |
| HG1018532 | 25058745_ECD | ig | (76-137) |
| HG1018532 | 25058745_ECD | ig | (175-248) |
| HG1018533 | 27260913_ECD | ig | (172-233) |
| HG1018533 | 27260913_ECD | ig | (55-109) |
| HG1018534 | NP_075258_ECD | ig | (172-233) |
| HG1018534 | NP_075258_ECD | ig | (271-342) |
| HG1018534 | NP_075258_ECD | ig | (55-109) |
| HG1018535 | NP_075261_ECD | ig | (172-233) |
| HG1018535 | NP_075261_ECD | ig | (271-347) |
| HG1018535 | NP_075261_ECD | ig | (55-109) |
| HG1018536 | NP_075262_ECD | ig | (172-233) |
| HG1018536 | NP_075262_ECD | ig | (271-344) |
| HG1018536 | NP_075262_ECD | ig | (55-109) |
| HG1018537 | NP_075264_ECD | ig | (83-144) |
| HG1018537 | NP_075264_ECD | ig | (182-253) |
| HG1018538 | NP_075418_ECD | ig | (83-144) |
| HG1018538 | NP_075418_ECD | ig | (182-255) |
| HG1018539 | NP_075419_ECD | ig | (57-118) |
| HG1018539 | NP_075419_ECD | ig | (156-227) |
| HG1018545 | 20452380_ECD | ig | (132-193) |
| HG1018545 | 20452380_ECD | ig | (231-304) |
| HG1018545 | 20452380_ECD | ig | (17-74) |
| HG1018546 | 20452381_ECD | ig | (132-193) |
| HG1018546 | 20452381_ECD | ig | (231-303) |
| HG1018546 | 20452381_ECD | ig | (17-74) |
| HG1018547 | 4503711_ECD | ig | (169-230) |
| HG1018547 | 4503711_ECD | ig | (268-341) |
| HG1018547 | 4503711_ECD | ig | (54-111) |
| HG1018548 | NovelFGFR3_clone021_ECD | ig | (169-230) |
| HG1018548 | NovelFGFR3_clone021_ECD | ig | (268-340) |
| HG1018548 | NovelFGFR3_clone021_ECD | ig | (54-111) |
| HG1018553 | 2832350_ECD | ig | (165-226) |
| HG1018553 | 2832350_ECD | ig | (264-335) |
| HG1018553 | 2832350_ECD | ig | (65-103) |
| HG1018554 | 7018380_ECD | ig | (22-93) |
| HG1018555 | NP_002002_ECD | ig | (165-226) |
| HG1018555 | NP_002002_ECD | ig | (264-335) |
| HG1018555 | NP_002002_ECD | ig | (65-103) |
| HG1018556 | proteinkinase113A_ECD | ig | (165-226) |
| HG1018556 | proteinkinase113A_ECD | ig | (264-335) |
| HG1018556 | proteinkinase113A_ECD | ig | (65-103) |

TABLE 5
Expression of FGFR1 in Human Tumors

| | | Tumors | | |
|---|---|---|---|---|
| Tumors | | Expressing | | |
| Searched | % FGFR1 | FGFR1 | Sample Site | Pathology/Morphology |
| 1 | 100 | 1 | Abdominal lymph node | Cholangiocarcinoma |
| 3 | 100 | 3 | Abdominal lymph node | Diffuse large B-cell |
| | | | | lymphoma |
| 1 | 100 | 1 | Abdominal lymph node | Hodgkin lymphoma, |
| | | | | nodular lymphocyte |
| | | | | predominance |
| 1 | 100 | 1 | Abdominal lymph node | Malignant melanoma |
| 1 | 100 | 1 | Abdominal lymph node | Mantle cell lymphoma |
| 1 | 100 | 1 | Abdominal lymph node | Signet ring cell |
| | | | | carcinoma |
| 1 | 100 | 1 | Adrenal gland | Carcinoma |
| 1 | 100 | 1 | Adrenal gland | Malignant melanoma |
| 1 | 100 | 1 | Adrenal medulla | Neuroblastoma |
| 1 | 100 | 1 | Anus | Malignant melanoma |
| 1 | 100 | 1 | Anus | Squamous cell |
| | | | | carcinoma |
| 1 | 100 | 1 | Appendix | Mullerian mixed tumor |
| 1 | 100 | 1 | Appendix | Neuroendocrine |
| | | | | carcinoma |
| 2 | 100 | 2 | Axillary lymph node | Carcinoma |
| 12 | 100 | 12 | Axillary lymph node | Infiltrating duct |
| | | | | carcinoma |
| 1 | 100 | 1 | Axillary lymph node | Infiltrating lobular |
| | | | | carcinoma |
| 1 | 100 | 1 | Axillary lymph node | Peripheral T-cell |
| | | | | lymphoma |
| 1 | 100 | 1 | Bladder | Carcinoma |
| 1 | 100 | 1 | Bladder | Mucinous |
| | | | | adenocarcinoma |
| 1 | 100 | 1 | Bone marrow | Multiple myeloma |
| 1 | 100 | 1 | Bone structure | Adenocarcinoma |
| 2 | 100 | 2 | Bone structure | Chondrosarcoma |
| 1 | 100 | 1 | Bone structure | Ewing's sarcoma |
| 4 | 100 | 4 | Bone structure | Osteosarcoma |
| 1 | 100 | 1 | Bone structure | Peripheral T-cell |
| | | | | lymphoma |
| 1 | 100 | 1 | Bone structure | Sarcoma |
| 2 | 100 | 2 | Brain | Astrocytoma |
| 2 | 100 | 2 | Brain | Diffuse large B-cell |
| | | | | lymphoma |
| 1 | 100 | 1 | Brain | Glioblastoma with |
| | | | | sarcomatous component |
| 1 | 100 | 1 | Brain | Malignant glioma |
| 3 | 100 | 3 | Brain | Medulloblastoma |
| 1 | 100 | 1 | Brain | Meningioma, malignant |
| 1 | 100 | 1 | Brain | Squamous cell |
| | | | | carcinoma |
| 1 | 100 | 1 | Breast | Angiosarcoma |
| 2 | 100 | 2 | Breast | Carcinoma |
| 2 | 100 | 2 | Breast | Medullary carcinoma |
| 4 | 100 | 4 | Breast | Mucinous |
| | | | | adenocarcinoma |
| 2 | 100 | 2 | Breast | Papillary |
| | | | | adenocarcinoma |
| 1 | 100 | 1 | Cardia of stomach | Adenocarcinoma |
| 1 | 100 | 1 | Cecum | Carcinoma |
| 1 | 100 | 1 | Cecum | Extranodal marginal |
| | | | | zone B-cell lymphoma of |
| | | | | mucosa-associated |
| | | | | lymphoid tissue |
| 1 | 100 | 1 | Cecum | Hepatocellular |
| | | | | carcinoma |
| 5 | 100 | 5 | Cecum | Mucinous |
| | | | | adenocarcinoma |
| 1 | 100 | 1 | Cecum | Papillary serous |
| | | | | adenocarcinoma |
| 1 | 100 | 1 | Cell | Mullerian mixed tumor |
| 1 | 100 | 1 | Cerebellum | Adenocarcinoma |
| 1 | 100 | 1 | Cervical lymph node | Carcinoma, anaplastic |
| 1 | 100 | 1 | Cervical lymph node | Histiocytic sarcoma |
| 1 | 100 | 1 | Cervical lymph node | Hodgkin lymphoma, |
| | | | | lymphocyte depletion |
| 1 | 100 | 1 | Cervical lymph node | Hodgkin lymphoma, |
| | | | | nodular sclerosis |
| 1 | 100 | 1 | Cervical lymph node | Nodal marginal zone B- |
| | | | | cell lymphoma |
| 3 | 100 | 3 | Cervical lymph node | Papillary carcinoma |
| 1 | 100 | 1 | Cervix | Endometrioid |
| | | | | adenocarcinoma |
| 1 | 100 | 1 | Colon | Dedifferentiated |
| | | | | liposarcoma |
| 1 | 100 | 1 | Colon | Diffuse large B-cell |
| | | | | lymphoma |
| 1 | 100 | 1 | Colon | Leiomyosarcoma |
| 1 | 100 | 1 | Colon | Mantle cell lymphoma |
| 2 | 100 | 2 | Colon | Papillary serous |
| | | | | adenocarcinoma |
| 1 | 100 | 1 | Colon | Serous |
| | | | | cystadenocarcinoma |
| 1 | 100 | 1 | Descending colon | Signet ring cell |
| | | | | carcinoma |
| 1 | 100 | 1 | Duodenum | Leiomyosarcoma |
| 1 | 100 | 1 | Duodenum | Mucinous |
| | | | | adenocarcinoma |
| 1 | 100 | 1 | Duodenum | Neuroendocrine |
| | | | | carcinoma |
| 1 | 100 | 1 | Duodenum | Signet ring cell |
| | | | | carcinoma |
| 2 | 100 | 2 | Endometrium | Adenoacanthoma |
| 10 | 100 | 10 | Endometrium | Adenocarcinoma |
| 1 | 100 | 1 | Endometrium | Adenosquamous |
| | | | | carcinoma |
| 1 | 100 | 1 | Endometrium | Carcinoma |
| 2 | 100 | 2 | Endometrium | Clear cell |
| | | | | adenocarcinoma |
| 11 | 100 | 11 | Endometrium | Mullerian mixed tumor |
| 1 | 100 | 1 | Endometrium | Neoplasm, malignant |
| 2 | 100 | 2 | Epithelial cell | Clear cell |
| | | | | adenocarcinoma |
| 1 | 100 | 1 | Epithelial cell | Renal cell carcinoma |
| 1 | 100 | 1 | Exocervix | Adenocarcinoma |
| 1 | 100 | 1 | Gallbladder | Follicular lymphoma |
| 4 | 100 | 4 | Gastroesophageal junction | Adenocarcinoma |
| 2 | 100 | 2 | Glial cell | Astrocytoma |
| 1 | 100 | 1 | Glial cell | Glioblastoma with |
| | | | | sarcomatous component |
| 7 | 100 | 7 | Glial cell | Malignant glioma |
| 1 | 100 | 1 | Heart | Fibromyxosarcoma |
| 1 | 100 | 1 | Ileum | Leiomyosarcoma |
| 1 | 100 | 1 | Ileum | Mucinous |
| | | | | adenocarcinoma |
| 1 | 100 | 1 | Inguinal lymph node | Adenocarcinoma |
| 1 | 100 | 1 | Inguinal lymph node | Chronic lymphocytic |
| | | | | leukemia/small |
| | | | | lymphocytic lymphoma |
| 2 | 100 | 2 | Inguinal lymph node | Follicular lymphoma |
| 1 | 100 | 1 | Inguinal lymph node | Mycosis fungoides |
| 1 | 100 | 1 | Inguinal lymph node | Nodal marginal zone B- |
| | | | | cell lymphoma |
| 2 | 100 | 2 | Jejunum | Adenocarcinoma |
| 1 | 100 | 1 | Jejunum | Malignant melanoma |
| 1 | 100 | 1 | Kidney | Carcinoma |
| 3 | 100 | 3 | Kidney | Chromophobe |
| | | | | carcinoma |
| 1 | 100 | 1 | Kidney | Diffuse large B-cell |
| | | | | lymphoma |
| 1 | 100 | 1 | Kidney | Neoplasm, malignant |
| 10 | 100 | 10 | Kidney | Wilms' tumor |
| 1 | 100 | 1 | Lacrimal gland | Squamous cell |
| | | | | carcinoma |
| 1 | 100 | 1 | Larynx | Adenoid cystic |
| | | | | carcinoma |
| 1 | 100 | 1 | Liver | Angiomyosarcoma |
| 1 | 100 | 1 | Liver | Fibrous histiocytoma, |
| | | | | malignant |
| 1 | 100 | 1 | Liver | Islet cell carcinoma |
| 1 | 100 | 1 | Liver | Malignant melanoma |
| 1 | 100 | 1 | Liver | Meningioma, malignant |
| 1 | 100 | 1 | Liver | Mucinous |
| | | | | adenocarcinoma |
| 1 | 100 | 1 | Liver | Papillary serous |
| | | | | adenocarcinoma |
| 1 | 100 | 1 | Liver | Renal cell carcinoma |
| 2 | 100 | 2 | Lung | Adenoid cystic |
| | | | | carcinoma |
| 1 | 100 | 1 | Lung | Choriocarcinoma |
| 2 | 100 | 2 | Lung | Clear cell |
| | | | | adenocarcinoma |
| 1 | 100 | 1 | Lung | Hurthle cell carcinoma |
| 3 | 100 | 3 | Lung | Leiomyosarcoma |
| 1 | 100 | 1 | Lung | Malignant lymphoma |
| 2 | 100 | 2 | Lung | Malignant melanoma |
| 1 | 100 | 1 | Lung | Osteosarcoma |
| 1 | 100 | 1 | Lung | Papillary |
| | | | | adenocarcinoma |
| 1 | 100 | 1 | Lung | Synovial sarcoma |
| 2 | 100 | 2 | Lymph node | Papillary carcinoma |
| 1 | 100 | 1 | Lymph node | Cholangiocarcinoma |
| 1 | 100 | 1 | Lymph node | Clear cell |
| | | | | adenocarcinoma |
| 1 | 100 | 1 | Lymph node | Fibrous histiocytoma, |
| | | | | malignant |
| 3 | 100 | 3 | Lymph node | Infiltrating duct |
| | | | | carcinoma |
| 1 | 100 | 1 | Lymph node | Malignant lymphoma, |
| | | | | lymphoblastic |
| 1 | 100 | 1 | Lymph node | Nodal marginal zone B- |
| | | | | cell lymphoma |
| 1 | 100 | 1 | Lymph node | Peripheral T-cell |
| | | | | lymphoma |
| 1 | 100 | 1 | Lymph node | Sarcoma |
| 4 | 100 | 4 | Lymph node | Squamous cell |
| | | | | carcinoma |
| 2 | 100 | 2 | Lymph node of head | Diffuse large B-cell |
| | | | | lymphoma |
| 2 | 100 | 2 | Lymph node of head | Follicular lymphoma |
| 1 | 100 | 1 | Lymphatic system | Malignant lymphoma |
| 1 | 100 | 1 | Lymphoblast | Precursor cell |
| | | | | lymphoblastic leukemia |
| 1 | 100 | 1 | Maxilla | Small cell carcinoma |
| 1 | 100 | 1 | Mediastinal lymph node | Adenocarcinoma |
| 1 | 100 | 1 | Mediastinum | Carcinoma, anaplastic |
| 1 | 100 | 1 | Mediastinum | Endodermal sinus tumor |
| 2 | 100 | 2 | Mediastinum | Neuroblastoma |
| 1 | 100 | 1 | Mediastinum | Seminoma |
| 1 | 100 | 1 | Mesentery | Diffuse large B-cell |
| | | | | lymphoma |
| 1 | 100 | 1 | Mesentery of small intestine | Adenocarcinoma |
| 1 | 100 | 1 | Myometrium | Adenocarcinoma |
| 3 | 100 | 3 | Myometrium | Leiomyosarcoma |
| 4 | 100 | 4 | Myometrium | Mullerian mixed tumor |
| 1 | 100 | 1 | Myometrium | Papillary serous |
| | | | | adenocarcinoma |
| 2 | 100 | 2 | Myometrium | Squamous cell |
| | | | | carcinoma |
| 1 | 100 | 1 | Nasopharynx | Squamous cell |
| | | | | carcinoma |
| 1 | 100 | 1 | Omentum | Carcinoma |
| 1 | 100 | 1 | Omentum | Goblet cell carcinoid |
| 2 | 100 | 2 | Omentum | Infiltrating lobular |
| | | | | carcinoma |
| 3 | 100 | 3 | Omentum | Leiomyosarcoma |
| 4 | 100 | 4 | Omentum | Mucinous |
| | | | | adenocarcinoma |
| 5 | 100 | 5 | Omentum | Mullerian mixed tumor |
| 1 | 100 | 1 | Omentum | Papillary |
| | | | | adenocarcinoma |
| 1 | 100 | 1 | Omentum | Sarcoma |
| 5 | 100 | 5 | Omentum | Serous |
| | | | | cystadenocarcinoma |
| 1 | 100 | 1 | Omentum | Signet ring cell |
| | | | | carcinoma |
| 5 | 100 | 5 | Oral cavity | Squamous cell |
| | | | | carcinoma |
| 1 | 100 | 1 | Ovary | Adenosquamous |
| | | | | carcinoma |
| 6 | 100 | 6 | Ovary | Carcinoma |
| 10 | 100 | 10 | Ovary | Clear cell |
| | | | | adenocarcinoma |
| 1 | 100 | 1 | Ovary | Cystadenocarcinoma |
| 2 | 100 | 2 | Ovary | Dysgerminoma |
| 2 | 100 | 2 | Ovary | Follicular lymphoma |
| 1 | 100 | 1 | Ovary | Granulosa cell tumor, |
| | | | | malignant |
| 1 | 100 | 1 | Ovary | Infiltrating duct and |
| | | | | lobular carcinoma |
| 1 | 100 | 1 | Ovary | Infiltrating lobular |
| | | | | carcinoma |
| 3 | 100 | 3 | Ovary | Mucinous |
| | | | | adenocarcinoma |
| 5 | 100 | 5 | Ovary | Mullerian mixed tumor |
| 1 | 100 | 1 | Ovary | Signet ring cell |
| | | | | carcinoma |
| 1 | 100 | 1 | Ovary | Teratoma, malignant |
| 1 | 100 | 1 | Pancreas | Acinar cell carcinoma |
| 1 | 100 | 1 | Pancreas | Carcinoma |
| 1 | 100 | 1 | Pancreas | Clear cell |
| | | | | adenocarcinoma |
| 1 | 100 | 1 | Pancreas | Follicular lymphoma |
| 1 | 100 | 1 | Pancreas | Mucinous |
| | | | | adenocarcinoma |
| 1 | 100 | 1 | Parotid gland | Adenocarcinoma |
| 1 | 100 | 1 | Parotid gland | Adenoid cystic |
| | | | | carcinoma |
| 1 | 100 | 1 | Parotid gland | Basal cell |
| | | | | adenocarcinoma |
| 1 | 100 | 1 | Parotid gland | Carcinoma in |
| | | | | pleomorphic adenoma |
| 1 | 100 | 1 | Parotid gland | Fibrous histiocytoma, |
| | | | | malignant |
| 1 | 100 | 1 | Parotid gland | Mucoepidermoid |
| | | | | carcinoma |
| 1 | 100 | 1 | Pelvic lymph node | Malignant melanoma |
| 1 | 100 | 1 | Pelvic lymph node | Serous |
| | | | | cystadenocarcinoma |
| 1 | 100 | 1 | Periaortic lymph node | Hepatocellular |
| | | | | carcinoma |
| 1 | 100 | 1 | Periaortic lymph node | Wilms' tumor |
| 1 | 100 | 1 | Peritoneum | Brenner tumor, |
| | | | | malignant |
| 1 | 100 | 1 | Peritoneum | Endometrioid |
| | | | | adenocarcinoma |
| 1 | 100 | 1 | Peritoneum | Goblet cell carcinoid |
| 1 | 100 | 1 | Peritoneum | Mucinous |
| | | | | adenocarcinoma |
| 2 | 100 | 2 | Peritoneum | Mullerian mixed tumor |
| 1 | 100 | 1 | Peritoneum | Papillary serous |
| | | | | adenocarcinoma |
| 1 | 100 | 1 | Peritoneum | Sarcoma |
| 1 | 100 | 1 | Pleura | Chondrosarcoma |
| 6 | 100 | 6 | Pleura | Mesothelioma, |
| | | | | malignant |
| 1 | 100 | 1 | Prostate | Rhabdomyosarcoma |
| 1 | 100 | 1 | Salivary gland | Acinar cell carcinoma |
| 1 | 100 | 1 | Salivary gland | Extranodal marginal |
| | | | | zone B-cell lymphoma of |
| | | | | mucosa-associated |
| | | | | lymphoid tissue |
| 1 | 100 | 1 | Sigmoid colon | Adenosquamous |
| | | | | carcinoma |
| 1 | 100 | 1 | Sigmoid colon | Diffuse large B-cell |
| | | | | lymphoma |
| 1 | 100 | 1 | Sigmoid colon | Peripheral T-cell |
| | | | | lymphoma |
| 4 | 100 | 4 | Skin | Dermatofibrosarcoma |
| | | | | protuberans |
| 17 | 100 | 17 | Skin | Mycosis fungoides |
| 1 | 100 | 1 | Skin | Neoplasm, malignant |
| 1 | 100 | 1 | Small intestine | Extranodal marginal |
| | | | | zone B-cell lymphoma of |
| | | | | mucosa-associated |
| | | | | lymphoid tissue |
| 1 | 100 | 1 | Small intestine | Follicular lymphoma |
| 1 | 100 | 1 | Small intestine | Mantle cell lymphoma |
| 1 | 100 | 1 | Small intestine | Small cell carcinoma |
| 2 | 100 | 2 | Soft tissues | Angiosarcoma |
| 2 | 100 | 2 | Soft tissues | Carcinoma |
| 1 | 100 | 1 | Soft tissues | Carcinoma in |
| | | | | pleomorphic adenoma |
| 1 | 100 | 1 | Soft tissues | Carcinosarcoma |
| 4 | 100 | 4 | Soft tissues | Chondrosarcoma |
| 1 | 100 | 1 | Soft tissues | Clear cell |
| | | | | adenocarcinoma |
| 3 | 100 | 3 | Soft tissues | Dedifferentiated |
| | | | | liposarcoma |
| 1 | 100 | 1 | Soft tissues | Epithelial-myoepithelial |
| | | | | carcinoma |
| 1 | 100 | 1 | Soft tissues | Ewing's sarcoma |
| 1 | 100 | 1 | Soft tissues | Fibromyxosarcoma |
| 4 | 100 | 4 | Soft tissues | Fibrosarcoma |
| 18 | 100 | 18 | Soft tissues | Fibrous histiocytoma, |
| | | | | malignant |
| 1 | 100 | 1 | Soft tissues | Follicular |
| | | | | adenocarcinoma |
| 6 | 100 | 6 | Soft tissues | Granulosa cell tumor, |
| | | | | malignant |
| 1 | 100 | 1 | Soft tissues | Infiltrating duct |
| | | | | carcinoma |
| 9 | 100 | 9 | Soft tissues | Leiomyosarcoma |
| 4 | 100 | 4 | Soft tissues | Liposarcoma |
| 1 | 100 | 1 | Soft tissues | Mucoepidermoid |
| | | | | carcinoma |
| 7 | 100 | 7 | Soft tissues | Myxoid liposarcoma |
| 1 | 100 | 1 | Soft tissues | Neoplasm, malignant |
| 6 | 100 | 6 | Soft tissues | Osteosarcoma |
| 6 | 100 | 6 | Soft tissues | Papillary serous |
| | | | | adenocarcinoma |
| 3 | 100 | 3 | Soft tissues | Pleomorphic |
| | | | | liposarcoma |
| 1 | 100 | 1 | Soft tissues | Primitive |
| | | | | neuroectodermal tumor |
| 2 | 100 | 2 | Soft tissues | Renal cell carcinoma |
| 1 | 100 | 1 | Soft tissues | Seminoma |
| 6 | 100 | 6 | Soft tissues | Synovial sarcoma |
| 1 | 100 | 1 | Soft tissues | Transitional cell |
| | | | | carcinoma |
| 1 | 100 | 1 | Soft tissues | Wilms' tumor |
| 1 | 100 | 1 | Spleen | Adenocarcinoma |
| 1 | 100 | 1 | Spleen | Chronic myelomonocytic |
| | | | | leukemia |
| 1 | 100 | 1 | Spleen | Precursor B-cell |
| | | | | lymphoblastic leukemia |
| 1 | 100 | 1 | Stomach | Adenocarcinoid tumor |
| 1 | 100 | 1 | Stomach | Carcinoma |
| 1 | 100 | 1 | Stomach | Diffuse large B-cell |
| | | | | lymphoma |
| 1 | 100 | 1 | Stomach | Extranodal marginal |
| | | | | zone B-cell lymphoma of |
| | | | | mucosa-associated |
| | | | | lymphoid tissue |
| 1 | 100 | 1 | Testis | Embryonal carcinoma |
| 10 | 100 | 10 | Testis | Mixed germ cell tumor |
| 10 | 100 | 10 | Testis | Seminoma |
| 4 | 100 | 4 | Thymus | Thymoma, malignant |
| 1 | 100 | 1 | Thyroid gland | Diffuse large B-cell |
| | | | | lymphoma |
| 1 | 100 | 1 | Thyroid gland | Extranodal marginal |
| | | | | zone B-cell lymphoma of |
| | | | | mucosa-associated |
| | | | | lymphoid tissue |
| 3 | 100 | 3 | Thyroid gland | Follicular |
| | | | | adenocarcinoma |
| 1 | 100 | 1 | Thyroid gland | Follicular lymphoma |
| 2 | 100 | 2 | Thyroid gland | Hurthle cell carcinoma |
| 1 | 100 | 1 | Thyroid gland | Squamous cell |
| | | | | carcinoma |
| 5 | 100 | 5 | Tongue | Squamous cell |
| | | | | carcinoma |
| 1 | 100 | 1 | Tonsil | Diffuse large B-cell |
| | | | | lymphoma |
| 1 | 100 | 1 | Tonsil | Follicular lymphoma |
| 1 | 100 | 1 | Tonsil | Squamous cell |
| | | | | carcinoma |
| 2 | 100 | 2 | Trachea | Adenoid cystic |
| | | | | carcinoma |
| 1 | 100 | 1 | Trachea | Papillary carcinoma |
| 1 | 100 | 1 | Uterus | Adenocarcinoma |
| 1 | 100 | 1 | Uterus | Adenosquamous |
| | | | | carcinoma |
| 1 | 100 | 1 | Uterus | Carcinoma |
| 1 | 100 | 1 | Uterus | Sarcoma |
| 1 | 100 | 1 | Vulva | Malignant melanoma |
| 3 | 100 | 3 | White blood cell | Precursor T-cell |
| | | | | lymphoblastic leukemia |
| 79 | 97 | 77 | Endometrium | Endometrioid |
| | | | | adenocarcinoma |
| 31 | 97 | 30 | Breast | Infiltrating lobular |
| | | | | carcinoma |
| 279 | 96 | 267 | Breast | Infiltrating duct |
| | | | | carcinoma |
| 22 | 95 | 21 | Glial cell | Glioblastoma multiforme |
| 38 | 95 | 36 | Ovary | Papillary serous |
| | | | | adenocarcinoma |
| 19 | 95 | 18 | Thyroid gland | Papillary carcinoma |
| 18 | 94 | 17 | Esophagus | Adenocarcinoma |
| 18 | 94 | 17 | Ovary | Endometrioid |
| | | | | adenocarcinoma |
| 16 | 94 | 15 | Breast | Infiltrating duct and |
| | | | | lobular carcinoma |
| 14 | 93 | 13 | Vulva | Squamous cell |
| | | | | carcinoma |
| 61 | 92 | 56 | Kidney | Clear cell |
| | | | | adenocarcinoma |
| 36 | 92 | 33 | Omentum | Papillary serous |
| | | | | adenocarcinoma |
| 12 | 92 | 11 | Ovary | Adenocarcinoma |
| 12 | 92 | 11 | Stomach | Signet ring cell |
| | | | | carcinoma |
| 11 | 91 | 10 | Omentum | Adenocarcinoma |
| 11 | 91 | 10 | Skin | Basal cell carcinoma |
| 10 | 90 | 9 | Brain | Glioblastoma multiforme |
| 10 | 90 | 9 | Pancreas | Islet cell carcinoma |
| 10 | 90 | 9 | Skin | Malignant melanoma |
| 10 | 90 | 9 | Skin | Squamous cell |
| | | | | carcinoma |
| 10 | 90 | 9 | Soft tissues | Sarcoma |
| 28 | 89 | 25 | Kidney | Renal cell carcinoma |
| 9 | 89 | 8 | Bone marrow | Precursor T-cell |
| | | | | lymphoblastic leukemia |
| 8 | 88 | 7 | Ovary | Mucinous |
| | | | | cystadenocarcinoma |
| 72 | 86 | 62 | Lung | Squamous cell |
| | | | | carcinoma |
| 7 | 86 | 6 | Soft tissues | Malignant melanoma |
| 26 | 85 | 22 | Colon | Adenocarcinoma |
| 13 | 85 | 11 | Lymph node | Hodgkin lymphoma, |
| | | | | nodular sclerosis |
| 6 | 83 | 5 | Rectum | Mucinous |
| | | | | adenocarcinoma |
| 11 | 82 | 9 | Larynx | Squamous cell |
| | | | | carcinoma |
| 42 | 81 | 34 | Stomach | Adenocarcinoma |
| 91 | 80 | 73 | Lung | Adenocarcinoma |
| 5 | 80 | 4 | Ampulla of Vater | Adenocarcinoma |
| 5 | 80 | 4 | Axillary lymph node | Malignant melanoma |
| 10 | 80 | 8 | Descending colon | Adenocarcinoma |
| 15 | 80 | 12 | Epithelial cell | Carcinoma |
| 5 | 80 | 4 | Inguinal lymph node | Malignant melanoma |
| 5 | 80 | 4 | Lung | Adenosquamous |
| | | | | carcinoma |
| 5 | 80 | 4 | Lymph node | Diffuse large B-cell |
| | | | | lymphoma |
| 5 | 80 | 4 | Stomach | Mucinous |
| | | | | adenocarcinoma |
| 9 | 78 | 7 | Brain | Oligodendroglioma |
| 9 | 78 | 7 | Soft tissues | Adenocarcinoma |
| 4 | 75 | 3 | Axillary lymph node | Follicular lymphoma |
| 16 | 75 | 12 | Bladder | Transitional cell |
| | | | | carcinoma |
| 4 | 75 | 3 | Endocervix | Adenocarcinoma |
| 4 | 75 | 3 | Lung | Neuroendocrine |
| | | | | carcinoma |
| 4 | 75 | 3 | Spleen | Chronic myeloid |
| | | | | leukemia |
| 11 | 73 | 8 | Abdominal lymph node | Adenocarcinoma |
| 11 | 73 | 8 | Transverse colon | Adenocarcinoma |
| 14 | 71 | 10 | Cervix | Squamous cell |
| | | | | carcinoma |
| 13 | 69 | 9 | Lung | Carcinoma |
| 81 | 69 | 56 | Lymphoblast | Precursor T-cell |
| | | | | lymphoblastic leukemia |
| 16 | 69 | 11 | Cecum | Adenocarcinoma |
| 3 | 67 | 2 | Abdominal cavity | Papillary serous |
| | | | | adenocarcinoma |
| 3 | 67 | 2 | Adrenal cortex | Adrenal cortical |
| | | | | carcinoma |
| 3 | 67 | 2 | Brain | Adenocarcinoma |
| 12 | 67 | 8 | Cervical lymph node | Squamous cell |
| | | | | carcinoma |
| 3 | 67 | 2 | Colon | Mucinous |
| | | | | adenocarcinoma |
| 9 | 67 | 6 | Duodenum | Adenocarcinoma |
| 3 | 67 | 2 | Esophagus | Squamous cell |
| | | | | carcinoma |
| 3 | 67 | 2 | Fibroblast | Fibrosarcoma |
| 3 | 67 | 2 | Liver | Cholangiocarcinoma |
| 3 | 67 | 2 | Lymph node | Carcinoma |
| 3 | 67 | 2 | Parotid gland | Acinar cell carcinoma |
| 3 | 67 | 2 | Parotid gland | Squamous cell |
| | | | | carcinoma |
| 3 | 67 | 2 | Soft tissues | Malignant Schwannoma |
| 3 | 67 | 2 | Thyroid gland | Carcinoma, anaplastic |
| 35 | 66 | 23 | Ascending colon | Adenocarcinoma |
| 35 | 66 | 23 | White blood cell | Chronic lymphocytic |
| | | | | leukemia |
| 28 | 64 | 18 | Monocyte | Acute monocytic |
| | | | | leukemia |
| 16 | 63 | 10 | Bone marrow | Precursor B-cell |
| | | | | lymphoblastic leukemia |
| 8 | 63 | 5 | Lymph node | Follicular lymphoma |
| 16 | 63 | 10 | Thyroid gland | Papillary carcinoma, |
| | | | | follicular variant |
| 5 | 60 | 3 | Bone marrow | Acute promyelocytic |
| | | | | leukemia |
| 12 | 58 | 7 | Soft tissues | Squamous cell |
| | | | | carcinoma |
| 46 | 57 | 26 | Rectum | Adenocarcinoma |
| 2 | 50 | 1 | Ascending colon | Mucinous |
| | | | | adenocarcinoma |
| 2 | 50 | 1 | Axillary lymph node | Squamous cell |
| | | | | carcinoma |
| 2 | 50 | 1 | Bladder | Adenocarcinoma |
| 2 | 50 | 1 | Cervical lymph node | Adenocarcinoma |
| 8 | 50 | 4 | Cervical lymph node | Follicular lymphoma |
| 2 | 50 | 1 | Cervical lymph node | Malignant lymphoma |
| 2 | 50 | 1 | Cervix | Carcinoma |
| 4 | 50 | 2 | Endometrium | Papillary serous |
| | | | | adenocarcinoma |
| 2 | 50 | 1 | Inguinal lymph node | Squamous cell |
| | | | | carcinoma |
| 2 | 50 | 1 | Kidney | Transitional cell |
| | | | | carcinoma |
| 2 | 50 | 1 | Lung | Bronchiolo-alveolar |
| | | | | adenocarcinoma |
| 10 | 50 | 5 | Lung | Large cell carcinoma |
| 2 | 50 | 1 | Penis | Squamous cell |
| | | | | carcinoma |
| 2 | 50 | 1 | Salivary gland | Adenoid cystic |
| | | | | carcinoma |
| 2 | 50 | 1 | Sigmoid colon | Mucinous |
| | | | | adenocarcinoma |
| 2 | 50 | 1 | Soft tissues | Diffuse large B-cell |
| | | | | lymphoma |
| 2 | 50 | 1 | Soft tissues | Ganglioneuroblastoma |
| 2 | 50 | 1 | Soft tissues | Papillary carcinoma |
| 2 | 50 | 1 | Soft tissues | Round cell liposarcoma |
| 2 | 50 | 1 | Spleen | Follicular lymphoma |
| 2 | 50 | 1 | Thyroid gland | Medullary carcinoma |
| | | | | with amyloid stroma |
| 2 | 50 | 1 | Ureter | Transitional cell |
| | | | | carcinoma |
| 11 | 45 | 5 | Epithelial cell | Large cell carcinoma |
| 9 | 44 | 4 | Epithelial cell | Infiltrating duct |
| | | | | carcinoma |
| 129 | 43 | 56 | Epithelial cell | Adenocarcinoma |
| 31 | 42 | 13 | Sigmoid colon | Adenocarcinoma |
| 5 | 40 | 2 | Lung | Small cell carcinoma |
| 8 | 38 | 3 | Lymph node | Malignant lymphoma |
| 12 | 33 | 4 | Bone marrow | Chronic myeloid |
| | | | | leukemia |
| 3 | 33 | 1 | Cervical lymph node | Mantle cell lymphoma |
| 6 | 33 | 2 | Epithelial cell | Squamous cell |
| | | | | carcinoma |
| 3 | 33 | 1 | Inguinal lymph node | Mantle cell lymphoma |
| 27 | 33 | 9 | Liver | Adenocarcinoma |
| 3 | 33 | 1 | Liver | Hepatoblastoma |
| 3 | 33 | 1 | Lymph node | Adenocarcinoma |
| 3 | 33 | 1 | Lymph node | Malignant melanoma |
| 3 | 33 | 1 | Small intestine | Diffuse large B-cell |
| | | | | lymphoma |
| 22 | 32 | 7 | Liver | Hepatocellular |
| | | | | carcinoma |
| 19 | 26 | 5 | Melanocyte | Malignant melanoma |
| 4 | 25 | 1 | Axillary lymph node | Diffuse large B-cell |
| | | | | lymphoma |
| 4 | 25 | 1 | Renal pelvis | Transitional cell |
| | | | | carcinoma |
| 13 | 23 | 3 | Ovary | Serous |
| | | | | cystadenocarcinoma |
| 5 | 20 | 1 | Spleen | Diffuse large B-cell |
| | | | | lymphoma |
| 28 | 14 | 4 | Promyelocyte | Acute promyelocytic |
| | | | | leukemia |
| 57 | 11 | 6 | Pancreas | Adenocarcinoma |
| 10 | 10 | 1 | Cervical lymph node | Diffuse large B-cell |
| | | | | lymphoma |
| 114 | 2 | 2 | Prostate | Adenocarcinoma |
| 5 | 0 | | White blood cell | Chronic myeloid |
| | | | | leukemia |

TABLE 6
Expression of FGFR2 in Human Tumors

| | | Tumors | | |
|---|---|---|---|---|
| Tumors | | Expressing | | |
| Searched | % FGFR2 | FGFR2 | Sample Site | Pathology/Morphology |
| 1 | 100 | 1 | Abdominal cavity | Adenocarcinoma |
| 1 | 100 | 1 | Abdominal cavity | Carcinoma |
| 3 | 100 | 3 | Abdominal cavity | Papillary serous |
| | | | | adenocarcinoma |
| 1 | 100 | 1 | Abdominal lymph node | Cholangiocarcinoma |
| 1 | 100 | 1 | Abdominal lymph node | Hepatocellular |
| | | | | carcinoma |
| 1 | 100 | 1 | Abdominal lymph node | Mantle cell lymphoma |
| 1 | 100 | 1 | Abdominal lymph node | Mucinous |
| | | | | adenocarcinoma |
| 1 | 100 | 1 | Adrenal gland | Renal cell carcinoma |
| 1 | 100 | 1 | Anus | Squamous cell |
| | | | | carcinoma |
| 1 | 100 | 1 | Appendix | Mullerian mixed tumor |
| 1 | 100 | 1 | Appendix | Neuroendocrine |
| | | | | carcinoma |
| 2 | 100 | 2 | Ascending colon | Mucinous |
| | | | | adenocarcinoma |
| 1 | 100 | 1 | Axillary lymph node | Infiltrating lobular |
| | | | | carcinoma |
| 1 | 100 | 1 | Axillary lymph node | Peripheral T-cell |
| | | | | lymphoma |
| 2 | 100 | 2 | Axillary lymph node | Squamous cell |
| | | | | carcinoma |
| 2 | 100 | 2 | Bladder | Adenocarcinoma |
| 1 | 100 | 1 | Bladder | Carcinoma |
| 1 | 100 | 1 | Bladder | Mucinous |
| | | | | adenocarcinoma |
| 1 | 100 | 1 | Bone structure | Adenocarcinoma |
| 2 | 100 | 2 | Bone structure | Chondrosarcoma |
| 1 | 100 | 1 | Bone structure | Sarcoma |
| 3 | 100 | 3 | Brain | Adenocarcinoma |
| 1 | 100 | 1 | Brain | Malignant glioma |
| 1 | 100 | 1 | Breast | Angiosarcoma |
| 2 | 100 | 2 | Breast | Papillary |
| | | | | adenocarcinoma |
| 1 | 100 | 1 | Cardia of stomach | Adenocarcinoma |
| 1 | 100 | 1 | Cecum | Extranodal marginal |
| | | | | zone B-cell lymphoma |
| | | | | of mucosa-associated |
| | | | | lymphoid tissue |
| 1 | 100 | 1 | Cecum | Papillary serous |
| | | | | adenocarcinoma |
| 1 | 100 | 1 | Cell | Mullerian mixed tumor |
| 1 | 100 | 1 | Cerebellum | Adenocarcinoma |
| 2 | 100 | 2 | Cervical lymph node | Adenocarcinoma |
| 3 | 100 | 3 | Cervical lymph node | Papillary carcinoma |
| 1 | 100 | 1 | Cervical lymph node | Small cell carcinoma |
| 2 | 100 | 2 | Cervix | Carcinoma |
| 1 | 100 | 1 | Cervix | Endometrioid |
| | | | | adenocarcinoma |
| 1 | 100 | 1 | Colon | Dedifferentiated |
| | | | | liposarcoma |
| 1 | 100 | 1 | Colon | Leiomyosarcoma |
| 3 | 100 | 3 | Colon | Mucinous |
| | | | | adenocarcinoma |
| 2 | 100 | 2 | Colon | Papillary serous |
| | | | | adenocarcinoma |
| 1 | 100 | 1 | Colon | Serous |
| | | | | cystadenocarcinoma |
| 1 | 100 | 1 | Descending colon | Signet ring cell |
| | | | | carcinoma |
| 1 | 100 | 1 | Duodenum | Leiomyosarcoma |
| 1 | 100 | 1 | Duodenum | Mucinous |
| | | | | adenocarcinoma |
| 1 | 100 | 1 | Duodenum | Neuroendocrine |
| | | | | carcinoma |
| 1 | 100 | 1 | Duodenum | Signet ring cell |
| | | | | carcinoma |
| 4 | 100 | 4 | Endocervix | Adenocarcinoma |
| 2 | 100 | 2 | Endometrium | Adenoacanthoma |
| 1 | 100 | 1 | Endometrium | Carcinoma |
| 1 | 100 | 1 | Epithelial cell | Clear cell |
| | | | | adenocarcinoma |
| 1 | 100 | 1 | Epithelial cell | Renal cell carcinoma |
| 3 | 100 | 3 | Esophagus | Squamous cell |
| | | | | carcinoma |
| 1 | 100 | 1 | Exocervix | Adenocarcinoma |
| 4 | 100 | 4 | Gastroesophageal junction | Adenocarcinoma |
| 1 | 100 | 1 | Glial cell | Glioblastoma with |
| | | | | sarcomatous |
| | | | | component |
| 1 | 100 | 1 | Heart | Fibromyxosarcoma |
| 1 | 100 | 1 | Ileum | Leiomyosarcoma |
| 1 | 100 | 1 | Ileum | Mucinous |
| | | | | adenocarcinoma |
| 1 | 100 | 1 | Inguinal lymph node | Mycosis fungoides |
| 1 | 100 | 1 | Kidney | Carcinoma |
| 2 | 100 | 2 | Kidney | Transitional cell |
| | | | | carcinoma |
| 10 | 100 | 10 | Kidney | Wilms' tumor |
| 1 | 100 | 1 | Larynx | Adenoid cystic |
| | | | | carcinoma |
| 11 | 100 | 11 | Larynx | Squamous cell |
| | | | | carcinoma |
| 1 | 100 | 1 | Liver | Angiomyosarcoma |
| 3 | 100 | 3 | Liver | Cholangiocarcinoma |
| 1 | 100 | 1 | Liver | Fibrous histiocytoma, |
| | | | | malignant |
| 1 | 100 | 1 | Liver | Meningioma, malignant |
| 1 | 100 | 1 | Liver | Mucinous |
| | | | | adenocarcinoma |
| 1 | 100 | 1 | Liver | Papillary serous |
| | | | | adenocarcinoma |
| 1 | 100 | 1 | Liver | Transitional cell |
| | | | | carcinoma |
| 2 | 100 | 2 | Lung | Adenoid cystic |
| | | | | carcinoma |
| 2 | 100 | 2 | Lung | Bronchiolo-alveolar |
| | | | | adenocarcinoma |
| 1 | 100 | 1 | Lung | Choriocarcinoma |
| 2 | 100 | 2 | Lung | Clear cell |
| | | | | adenocarcinoma |
| 1 | 100 | 1 | Lung | Hurthle cell carcinoma |
| 3 | 100 | 3 | Lung | Leiomyosarcoma |
| 1 | 100 | 1 | Lung | Malignant lymphoma |
| 1 | 100 | 1 | Lung | Osteosarcoma |
| 1 | 100 | 1 | Lung | Papillary |
| | | | | adenocarcinoma |
| 1 | 100 | 1 | Lung | Synovial sarcoma |
| 1 | 100 | 1 | Lymph node | Cholangiocarcinoma |
| 1 | 100 | 1 | Lymph node | Clear cell |
| | | | | adenocarcinoma |
| 1 | 100 | 1 | Lymph node | Papillary carcinoma |
| 1 | 100 | 1 | Lymph node | Peripheral T-cell |
| | | | | lymphoma |
| 1 | 100 | 1 | Maxilla | Squamous cell |
| | | | | carcinoma |
| 1 | 100 | 1 | Mediastinal lymph node | Squamous cell |
| | | | | carcinoma |
| 1 | 100 | 1 | Mediastinum | Neuroendocrine |
| | | | | carcinoma |
| 1 | 100 | 1 | Megakaryocyte | Acute megakaryoblastic |
| | | | | leukemia |
| 1 | 100 | 1 | Mesentery of small intestine | Adenocarcinoma |
| 1 | 100 | 1 | Myometrium | Adenocarcinoma |
| 3 | 100 | 3 | Myometrium | Leiomyosarcoma |
| 4 | 100 | 4 | Myometrium | Mullerian mixed tumor |
| 1 | 100 | 1 | Myometrium | Papillary serous |
| | | | | adenocarcinoma |
| 2 | 100 | 2 | Myometrium | Squamous cell |
| | | | | carcinoma |
| 1 | 100 | 1 | Nasopharynx | Squamous cell |
| | | | | carcinoma |
| 1 | 100 | 1 | Omentum | Goblet cell carcinoid |
| 1 | 100 | 1 | Omentum | Papillary |
| | | | | adenocarcinoma |
| 1 | 100 | 1 | Omentum | Sarcoma |
| 5 | 100 | 5 | Omentum | Serous |
| | | | | cystadenocarcinoma |
| 1 | 100 | 1 | Omentum | Signet ring cell |
| | | | | carcinoma |
| 1 | 100 | 1 | Ovary | Adenosquamous |
| | | | | carcinoma |
| 6 | 100 | 6 | Ovary | Carcinoma |
| 10 | 100 | 10 | Ovary | Clear cell |
| | | | | adenocarcinoma |
| 1 | 100 | 1 | Ovary | Cystadenocarcinoma |
| 1 | 100 | 1 | Ovary | Teratoma, malignant |
| 1 | 100 | 1 | Pancreas | Acinar cell carcinoma |
| 1 | 100 | 1 | Pancreas | Carcinoma |
| 1 | 100 | 1 | Pancreas | Clear cell |
| | | | | adenocarcinoma |
| 1 | 100 | 1 | Pancreas | Mucinous |
| | | | | adenocarcinoma |
| 3 | 100 | 3 | Parotid gland | Acinar cell carcinoma |
| 1 | 100 | 1 | Parotid gland | Adenocarcinoma |
| 1 | 100 | 1 | Parotid gland | Adenoid cystic |
| | | | | carcinoma |
| 1 | 100 | 1 | Parotid gland | Basal cell |
| | | | | adenocarcinoma |
| 1 | 100 | 1 | Parotid gland | Carcinoma in |
| | | | | pleomorphic adenoma |
| 1 | 100 | 1 | Pelvic lymph node | Malignant melanoma |
| 1 | 100 | 1 | Pelvic lymph node | Serous |
| | | | | cystadenocarcinoma |
| 2 | 100 | 2 | Penis | Squamous cell |
| | | | | carcinoma |
| 1 | 100 | 1 | Periaortic lymph node | Wilms' tumor |
| 1 | 100 | 1 | Peritoneum | Brenner tumor, |
| | | | | malignant |
| 1 | 100 | 1 | Peritoneum | Endometrioid |
| | | | | adenocarcinoma |
| 1 | 100 | 1 | Peritoneum | Goblet cell carcinoid |
| 1 | 100 | 1 | Peritoneum | Mucinous |
| | | | | adenocarcinoma |
| 2 | 100 | 2 | Peritoneum | Mullerian mixed tumor |
| 1 | 100 | 1 | Peritoneum | Papillary serous |
| | | | | adenocarcinoma |
| 1 | 100 | 1 | Peritoneum | Sarcoma |
| 1 | 100 | 1 | Pleura | Chondrosarcoma |
| 6 | 100 | 6 | Rectum | Mucinous |
| | | | | adenocarcinoma |
| 1 | 100 | 1 | Salivary gland | Acinar cell carcinoma |
| 2 | 100 | 2 | Salivary gland | Adenoid cystic |
| | | | | carcinoma |
| 11 | 100 | 11 | Skin | Basal cell carcinoma |
| 1 | 100 | 1 | Small intestine | Follicular lymphoma |
| 1 | 100 | 1 | Soft tissues | Alveolar soft part |
| | | | | sarcoma |
| 1 | 100 | 1 | Soft tissues | Carcinoma in |
| | | | | pleomorphic adenoma |
| 1 | 100 | 1 | Soft tissues | Carcinosarcoma |
| 2 | 100 | 2 | Soft tissues | Desmoplastic small |
| | | | | round cell tumor |
| 1 | 100 | 1 | Soft tissues | Follicular |
| | | | | adenocarcinoma |
| 1 | 100 | 1 | Soft tissues | Infiltrating duct |
| | | | | carcinoma |
| 1 | 100 | 1 | Soft tissues | Mucoepidermoid |
| | | | | carcinoma |
| 2 | 100 | 2 | Soft tissues | Papillary carcinoma |
| 6 | 100 | 6 | Soft tissues | Papillary serous |
| | | | | adenocarcinoma |
| 1 | 100 | 1 | Soft tissues | Primitive |
| | | | | neuroectodermal tumor |
| 1 | 100 | 1 | Soft tissues | Seminoma |
| 6 | 100 | 6 | Soft tissues | Synovial sarcoma |
| 1 | 100 | 1 | Soft tissues | Transitional cell |
| | | | | carcinoma |
| 1 | 100 | 1 | Soft tissues | Wilms' tumor |
| 1 | 100 | 1 | Spleen | Adenocarcinoma |
| 1 | 100 | 1 | Spleen | Hepatocellular |
| | | | | carcinoma |
| 1 | 100 | 1 | Spleen | Serous |
| | | | | cystadenocarcinoma |
| 1 | 100 | 1 | Stomach | Carcinoma |
| 1 | 100 | 1 | Stomach | Extranodal marginal |
| | | | | zone B-cell lymphoma |
| | | | | of mucosa-associated |
| | | | | lymphoid tissue |
| 12 | 100 | 12 | Stomach | Signet ring cell |
| | | | | carcinoma |
| 1 | 100 | 1 | Testis | Embryonal carcinoma |
| 10 | 100 | 10 | Testis | Seminoma |
| 1 | 100 | 1 | Thyroid gland | Extranodal marginal |
| | | | | zone B-cell lymphoma |
| | | | | of mucosa-associated |
| | | | | lymphoid tissue |
| 3 | 100 | 3 | Thyroid gland | Follicular |
| | | | | adenocarcinoma |
| 2 | 100 | 2 | Thyroid gland | Hurthle cell carcinoma |
| 19 | 100 | 19 | Thyroid gland | Papillary carcinoma |
| 1 | 100 | 1 | Tonsil | Follicular lymphoma |
| 1 | 100 | 1 | Tonsil | Squamous cell |
| | | | | carcinoma |
| 2 | 100 | 2 | Trachea | Adenoid cystic |
| | | | | carcinoma |
| 1 | 100 | 1 | Trachea | Papillary carcinoma |
| 2 | 100 | 2 | Ureter | Transitional cell |
| | | | | carcinoma |
| 1 | 100 | 1 | Uterus | Adenocarcinoma |
| 1 | 100 | 1 | Uterus | Adenosquamous |
| | | | | carcinoma |
| 1 | 100 | 1 | Uterus | Sarcoma |
| 1 | 100 | 1 | Uterus | Squamous cell |
| | | | | carcinoma |
| 18 | 94 | 17 | Esophagus | Adenocarcinoma |
| 36 | 94 | 34 | Omentum | Papillary serous |
| | | | | adenocarcinoma |
| 79 | 94 | 74 | Endometrium | Endometrioid |
| | | | | adenocarcinoma |
| 38 | 92 | 35 | Ovary | Papillary serous |
| | | | | adenocarcinoma |
| 11 | 91 | 10 | Omentum | Adenocarcinoma |
| 31 | 90 | 28 | Breast | Infiltrating lobular |
| | | | | carcinoma |
| 10 | 90 | 9 | Brain | Glioblastoma multiforme |
| 10 | 90 | 9 | Endometrium | Adenocarcinoma |
| 10 | 90 | 9 | Testis | Mixed germ cell tumor |
| 8 | 88 | 7 | Ovary | Mucinous |
| | | | | cystadenocarcinoma |
| 14 | 86 | 12 | Cervix | Squamous cell |
| | | | | carcinoma |
| 14 | 86 | 12 | Vulva | Squamous cell |
| | | | | carcinoma |
| 57 | 82 | 47 | Pancreas | Adenocarcinoma |
| 11 | 82 | 9 | Endometrium | Mullerian mixed tumor |
| 22 | 82 | 18 | Liver | Hepatocellular |
| | | | | carcinoma |
| 16 | 81 | 13 | Bladder | Transitional cell |
| | | | | carcinoma |
| 16 | 81 | 13 | Breast | Infiltrating duct and |
| | | | | lobular carcinoma |
| 5 | 80 | 4 | Lung | Adenosquamous |
| | | | | carcinoma |
| 5 | 80 | 4 | Stomach | Mucinous |
| | | | | adenocarcinoma |
| 5 | 80 | 4 | Tongue | Squamous cell |
| | | | | carcinoma |
| 9 | 78 | 7 | Brain | Oligodendroglioma |
| 18 | 78 | 14 | Ovary | Endometrioid |
| | | | | adenocarcinoma |
| 9 | 78 | 7 | Soft tissues | Leiomyosarcoma |
| 42 | 76 | 32 | Stomach | Adenocarcinoma |
| 4 | 75 | 3 | Bone structure | Osteosarcoma |
| 4 | 75 | 3 | Breast | Mucinous |
| | | | | adenocarcinoma |
| 4 | 75 | 3 | Endometrium | Papillary serous |
| | | | | adenocarcinoma |
| 4 | 75 | 3 | Lymph node | Squamous cell |
| | | | | carcinoma |
| 4 | 75 | 3 | Omentum | Mucinous |
| | | | | adenocarcinoma |
| 4 | 75 | 3 | Renal pelvis | Transitional cell |
| | | | | carcinoma |
| 4 | 75 | 3 | Soft tissues | Chondrosarcoma |
| 4 | 75 | 3 | Soft tissues | Fibrosarcoma |
| 7 | 71 | 5 | Soft tissues | Myxoid liposarcoma |
| 61 | 70 | 43 | kidney | Clear cell |
| | | | | adenocarcinoma |
| 27 | 70 | 19 | Liver | Adenocarcinoma |
| 10 | 70 | 7 | Lung | Large cell carcinoma |
| 10 | 70 | 7 | Skin | Squamous cell |
| | | | | carcinoma |
| 13 | 69 | 9 | Ovary | Serous |
| | | | | cystadenocarcinoma |
| 16 | 69 | 11 | Cecum | Adenocarcinoma |
| 3 | 67 | 2 | Abdominal lymph node | Diffuse large B-cell |
| | | | | lymphoma |
| 9 | 67 | 6 | Duodenum | Adenocarcinoma |
| 6 | 67 | 4 | Epithelial cell | Squamous cell |
| | | | | carcinoma |
| 3 | 67 | 2 | Fibroblast | Fibrosarcoma |
| 3 | 67 | 2 | Kidney | Chromophobe |
| | | | | carcinoma |
| 3 | 67 | 2 | Lymph node | Adenocarcinoma |
| 3 | 67 | 2 | Ovary | Mucinous |
| | | | | adenocarcinoma |
| 3 | 67 | 2 | Parotid gland | Squamous cell |
| | | | | carcinoma |
| 6 | 67 | 4 | Pleura | Mesothelioma, |
| | | | | malignant |
| 6 | 67 | 4 | Soft tissues | Osteosarcoma |
| 12 | 67 | 8 | Soft tissues | Squamous cell |
| | | | | carcinoma |
| 3 | 67 | 2 | Thyroid gland | Carcinoma, anaplastic |
| 129 | 66 | 85 | Epithelial cell | Adenocarcinoma |
| 114 | 66 | 75 | Prostate | Adenocarcinoma |
| 17 | 65 | 11 | Skin | Mycosis fungoides |
| 72 | 64 | 46 | Lung | Squamous cell |
| | | | | carcinoma |
| 28 | 61 | 17 | Kidney | Renal cell carcinoma |
| 5 | 60 | 3 | Cecum | Mucinous |
| | | | | adenocarcinoma |
| 10 | 60 | 6 | Descending colon | Adenocarcinoma |
| 5 | 60 | 3 | Lung | Small cell carcinoma |
| 5 | 60 | 3 | Omentum | Mullerian mixed tumor |
| 5 | 60 | 3 | Oral cavity | Squamous cell |
| | | | | carcinoma |
| 5 | 60 | 3 | Ovary | Mullerian mixed tumor |
| 31 | 58 | 18 | Sigmoid colon | Adenocarcinoma |
| 46 | 54 | 25 | Rectum | Adenocarcinoma |
| 91 | 54 | 49 | Lung | Adenocarcinoma |
| 13 | 54 | 7 | Lung | Carcinoma |
| 35 | 51 | 18 | Ascending colon | Adenocarcinoma |
| 2 | 50 | 1 | Brain | Astrocytoma |
| 2 | 50 | 1 | Breast | Carcinoma |
| 2 | 50 | 1 | Breast | Medullary carcinoma |
| 2 | 50 | 1 | Endometrium | Clear cell |
| | | | | adenocarcinoma |
| 2 | 50 | 1 | Glial cell | Astrocytoma |
| 2 | 50 | 1 | Inguinal lymph node | Squamous cell |
| | | | | carcinoma |
| 2 | 50 | 1 | Lung | Malignant melanoma |
| 4 | 50 | 2 | Lung | Neuroendocrine |
| | | | | carcinoma |
| 2 | 50 | 1 | Lymph node of head | Follicular lymphoma |
| 2 | 50 | 1 | Omentum | Infiltrating lobular |
| | | | | carcinoma |
| 12 | 50 | 6 | Ovary | Adenocarcinoma |
| 2 | 50 | 1 | Ovary | Follicular lymphoma |
| 2 | 50 | 1 | Sigmoid colon | Mucinous |
| | | | | adenocarcinoma |
| 4 | 50 | 2 | Skin | Dermatofibrosarcoma |
| | | | | protuberans |
| 10 | 50 | 5 | Skin | Malignant melanoma |
| 2 | 50 | 1 | Soft tissues | Ganglioneuroblastoma |
| 4 | 50 | 2 | Soft tissues | Liposarcoma |
| 2 | 50 | 1 | Soft tissues | Renal cell carcinoma |
| 2 | 50 | 1 | Soft tissues | Round cell liposarcoma |
| 10 | 50 | 5 | Soft tissues | Sarcoma |
| 4 | 50 | 2 | Thymus | Thymoma, malignant |
| 11 | 45 | 5 | Abdominal lymph node | Adenocarcinoma |
| 11 | 45 | 5 | Transverse colon | Adenocarcinoma |
| 9 | 44 | 4 | Soft tissues | Adenocarcinoma |
| 18 | 44 | 8 | Soft tissues | Fibrous histiocytoma, |
| | | | | malignant |
| 5 | 40 | 2 | Ampulla of Vater | Adenocarcinoma |
| 10 | 40 | 4 | Pancreas | Islet cell carcinoma |
| 26 | 35 | 9 | Colon | Adenocarcinoma |
| 3 | 33 | 1 | Brain | Medulloblastoma |
| 3 | 33 | 1 | Cervical lymph node | Mantle cell lymphoma |
| 9 | 33 | 3 | Epithelial cell | Infiltrating duct |
| | | | | carcinoma |
| 3 | 33 | 1 | Lymph node | Carcinoma |
| 3 | 33 | 1 | Lymph node | Infiltrating duct |
| | | | | carcinoma |
| 3 | 33 | 1 | Omentum | Leiomyosarcoma |
| 3 | 33 | 1 | Soft tissues | Malignant Schwannoma |
| 22 | 32 | 7 | Glial cell | Glioblastoma multiforme |
| 16 | 31 | 5 | Thyroid gland | Papillary carcinoma, |
| | | | | follicular variant |
| 7 | 29 | 2 | Glial cell | Malignant glioma |
| 15 | 27 | 4 | Epithelial cell | Carcinoma |
| 4 | 25 | 1 | Axillary lymph node | Diffuse large B-cell |
| | | | | lymphoma |
| 4 | 25 | 1 | Axillary lymph node | Follicular lymphoma |
| 12 | 25 | 3 | Axillary lymph node | Infiltrating duct |
| | | | | carcinoma |
| 5 | 20 | 1 | Bone marrow | Acute monocytic |
| | | | | leukemia |
| 5 | 20 | 1 | Inguinal lymph node | Malignant melanoma |
| 5 | 20 | 1 | Lymph node | Diffuse large B-cell |
| | | | | lymphoma |
| 11 | 18 | 2 | Epithelial cell | Large cell carcinoma |
| 8 | 13 | 1 | Cervical lymph node | Follicular lymphoma |
| 10 | 10 | 1 | Cervical lymph node | Diffuse large B-cell |
| | | | | lymphoma |
| 12 | 8 | 1 | Bone marrow | Chronic myeloid |
| | | | | leukemia |
| 12 | 8 | 1 | Cervical lymph node | Squamous cell |
| | | | | carcinoma |
| 279 | 4 | 12 | Breast | Infiltrating duct |
| | | | | carcinoma |
| 28 | 4 | 1 | Monocyte | Acute monocytic |
| | | | | leukemia |
| 80 | 1 | 1 | Lymphoblast | Burkitt's lymphoma |
| 3 | 0 | | Liver | Hepatoblastoma |
| 2 | 0 | | Ovary | Dysgerminoma |

TABLE 7
Expression of FGFR3 in Human Tumors

| | | Tumors | | |
|---|---|---|---|---|
| Tumors | | Expressing | | |
| Searched | % FGFR3 | FGFR3 | Sample Site | Pathology/Morphology |
| 1 | 100 | 1 | Abdominal | Mucinous adenocarcinoma |
| | | | lymph node | |
| 1 | 100 | 1 | Anus | Squamous cell carcinoma |
| 1 | 100 | 1 | Appendix | Mullerian mixed tumor |
| 1 | 100 | 1 | Bladder | Mucinous adenocarcinoma |
| 2 | 100 | 2 | Bone | Chondrosarcoma |
| | | | structure | |
| 4 | 100 | 4 | Bone | Osteosarcoma |
| | | | structure | |
| 2 | 100 | 2 | Brain | Astrocytoma |
| 1 | 100 | 1 | Breast | Angiosarcoma |
| 1 | 100 | 1 | Cardia of | Adenocarcinoma |
| | | | stomach | |
| 1 | 100 | 1 | Cervical | Small cell carcinoma |
| | | | lymph node | |
| 1 | 100 | 1 | Colon | Dedifferentiated liposarcoma |
| 1 | 100 | 1 | Endometrium | Adenosquamous carcinoma |
| 3 | 100 | 3 | Esophagus | Squamous cell carcinoma |
| 2 | 100 | 2 | Inguinal | Squamous cell carcinoma |
| | | | lymph node | |
| 1 | 100 | 1 | Kidney | Neoplasm, malignant |
| 2 | 100 | 2 | Kidney | Transitional cell carcinoma |
| 3 | 100 | 3 | Liver | Hepatoblastoma |
| 1 | 100 | 1 | Liver | Islet cell carcinoma |
| 1 | 100 | 1 | Liver | Transitional cell carcinoma |
| 2 | 100 | 2 | Lung | Bronchiolo-alveolar adenocarcinoma |
| 1 | 100 | 1 | Lung | Osteosarcoma |
| 1 | 100 | 1 | Lung | Papillary adenocarcinoma |
| 1 | 100 | 1 | Lung | Synovial sarcoma |
| 2 | 100 | 2 | Lymphoblast | Chronic myeloid leukemia |
| 1 | 100 | 1 | Maxilla | Squamous cell carcinoma |
| 1 | 100 | 1 | Mediastinal | Squamous cell carcinoma |
| | | | lymph node | |
| 1 | 100 | 1 | Mesentery of | Adenocarcinoma |
| | | | small intestine | |
| 1 | 100 | 1 | Myeloblast | Chronic myeloid leukemia |
| 1 | 100 | 1 | Nasopharynx | Squamous cell carcinoma |
| 1 | 100 | 1 | Omentum | Follicular lymphoma |
| 2 | 100 | 2 | Ovary | Dysgerminoma |
| 1 | 100 | 1 | Ovary | Infiltrating duct and lobular carcinoma |
| 1 | 100 | 1 | Ovary | Teratoma, malignant |
| 1 | 100 | 1 | Parotid gland | Carcinoma in pleomorphic adenoma |
| 1 | 100 | 1 | Pelvic lymph | Transitional cell carcinoma |
| | | | node | |
| 2 | 100 | 2 | Penis | Squamous cell carcinoma |
| 11 | 100 | 11 | Skin | Basal cell carcinoma |
| 1 | 100 | 1 | Soft tissues | Carcinoma in pleomorphic adenoma |
| 1 | 100 | 1 | Soft tissues | Epithelial-myoepithelial carcinoma |
| 1 | 100 | 1 | Soft tissues | Ewing's sarcoma |
| 1 | 100 | 1 | Soft tissues | Primitive neuroectodermal tumor |
| 1 | 100 | 1 | Soft tissues | Seminoma |
| 1 | 100 | 1 | Soft tissues | Transitional cell carcinoma |
| 1 | 100 | 1 | Spleen | Hepatocellular carcinoma |
| 1 | 100 | 1 | Stomach | Adenocarcinoid tumor |
| 1 | 100 | 1 | Testis | Embryonal carcinoma |
| 10 | 100 | 10 | Testis | Mixed germ cell tumor |
| 10 | 100 | 10 | Testis | Seminoma |
| 5 | 100 | 5 | Tongue | Squamous cell carcinoma |
| 1 | 100 | 1 | Tonsil | Squamous cell carcinoma |
| 2 | 100 | 2 | Ureter | Transitional cell carcinoma |
| 1 | 100 | 1 | Uterus | Adenosquamous carcinoma |
| 1 | 100 | 1 | Uterus | Squamous cell carcinoma |
| 14 | 93 | 13 | Vulva | Squamous cell carcinoma |
| 11 | 91 | 10 | Larynx | Squamous cell carcinoma |
| 10 | 90 | 9 | Skin | Squamous cell carcinoma |
| 6 | 83 | 5 | Soft tissues | Synovial sarcoma |
| 14 | 79 | 11 | Cervix | Squamous cell carcinoma |
| 72 | 76 | 55 | Lung | Squamous cell carcinoma |
| 16 | 75 | 12 | Bladder | Transitional cell carcinoma |
| 12 | 75 | 9 | Cervical | Squamous cell carcinoma |
| | | | lymph node | |
| 4 | 75 | 3 | Renal pelvis | Transitional cell carcinoma |
| 17 | 71 | 12 | Skin | Mycosis fungoides |
| 3 | 67 | 2 | Brain | Adenocarcinoma |
| 3 | 67 | 2 | Colon | Mucinous adenocarcinoma |
| 3 | 67 | 2 | Liver | Cholangiocarcinoma |
| 22 | 64 | 14 | Liver | Hepatocellular carcinoma |
| 18 | 61 | 11 | Esophagus | Adenocarcinoma |
| 10 | 60 | 6 | Brain | Glioblastoma multiforme |
| 12 | 58 | 7 | Soft tissues | Squamous cell carcinoma |
| 2 | 50 | 1 | Ascending | Mucinous adenocarcinoma |
| | | | colon | |
| 2 | 50 | 1 | Axillary lymph | Squamous cell carcinoma |
| | | | node | |
| 2 | 50 | 1 | Bone marrow | Chronic myeloid leukemia |
| 2 | 50 | 1 | Breast | Carcinoma |
| 2 | 50 | 1 | Endometrium | Clear cell adenocarcinoma |
| 4 | 50 | 2 | Endometrium | Papillary serous adenocarcinoma |
| 2 | 50 | 1 | Jejunum | Adenocarcinoma |
| 2 | 50 | 1 | Lung | Clear cell adenocarcinoma |
| 4 | 50 | 2 | Lymph node | Squamous cell carcinoma |
| 2 | 50 | 1 | Lymph node | Follicular lymphoma |
| | | | of head | |
| 10 | 50 | 5 | Ovary | Clear cell adenocarcinoma |
| 10 | 50 | 5 | Pancreas | Islet cell carcinoma |
| 6 | 50 | 3 | Soft tissues | Osteosarcoma |
| 2 | 50 | 1 | Soft tissues | Round cell liposarcoma |
| 9 | 44 | 4 | Brain | Oligodendroglioma |
| 16 | 44 | 7 | Breast | Infiltrating duct and lobular carcinoma |
| 26 | 42 | 11 | Colon | Adenocarcinoma; group as colorectal |
| | | | | cancer |
| 5 | 40 | 2 | Lung | Adenosquamous carcinoma |
| 5 | 40 | 2 | Omentum | Mullerian mixed tumor |
| 5 | 40 | 2 | Omentum | Serous cystadenocarcinoma |
| 5 | 40 | 2 | Oral cavity | Squamous cell carcinoma |
| 61 | 38 | 23 | Kidney | Clear cell adenocarcinoma |
| 8 | 38 | 3 | Ovary | Mucinous cystadenocarcinoma |
| 35 | 37 | 13 | Ascending | Adenocarcinoma; group as colorectal |
| | | | colon | cancer |
| 27 | 37 | 10 | Liver | Adenocarcinoma |
| 11 | 36 | 4 | Transverse | Adenocarcinoma; group as colorectal |
| | | | colon | cancer |
| 91 | 36 | 33 | Lung | Adenocarcinoma |
| 57 | 35 | 20 | Pancreas | Adenocarcinoma |
| 114 | 35 | 40 | Prostate | Adenocarcinoma |
| 6 | 33 | 2 | Epithelial cell | Squamous cell carcinoma |
| 3 | 33 | 1 | Omentum | Leiomyosarcoma |
| 3 | 33 | 1 | Parotid gland | Squamous cell carcinoma |
| 6 | 33 | 2 | Rectum | Mucinous adenocarcinoma |
| 3 | 33 | 1 | Soft tissues | Malignant Schwannoma |
| 10 | 30 | 3 | Skin | Malignant melanoma |
| 31 | 29 | 9 | Breast | Infiltrating lobular carcinoma |
| 31 | 29 | 9 | Sigmoid colon | Adenocarcinoma; group as colorectal |
| | | | | cancer |
| 129 | 29 | 37 | Epithelial cell | Adenocarcinoma |
| 279 | 25 | 70 | Breast | Infiltrating duct carcinoma |
| 4 | 25 | 1 | Breast | Mucinous adenocarcinoma |
| 4 | 25 | 1 | Lung | Neuroendocrine carcinoma |
| 4 | 25 | 1 | Soft tissues | Chondrosarcoma |
| 4 | 25 | 1 | Soft tissues | Fibrosarcoma |
| 4 | 25 | 1 | Spleen | Chronic myeloid leukemia |
| 42 | 24 | 10 | Stomach | Adenocarcinoma |
| 13 | 23 | 3 | Lung | Carcinoma |
| 9 | 22 | 2 | Duodenum | Adenocarcinoma |
| 9 | 22 | 2 | Epithelial cell | Infiltrating duct carcinoma |
| 9 | 22 | 2 | Soft tissues | Adenocarcinoma |
| 5 | 20 | 1 | Ampulla of | Adenocarcinoma |
| | | | Vater | |
| 10 | 20 | 2 | Endometrium | Adenocarcinoma |
| 10 | 20 | 2 | Lung | Large cell carcinoma |
| 5 | 20 | 1 | Lung | Small cell carcinoma |
| 5 | 20 | 1 | Ovary | Mullerian mixed tumor |
| 5 | 20 | 1 | Stomach | Mucinous adenocarcinoma |
| 81 | 20 | 16 | Lymphoblast | Precursor T-cell lymphoblastic |
| | | | | leukemia |
| 11 | 18 | 2 | Endometrium | Mullerian mixed tumor |
| 11 | 18 | 2 | Omentum | Adenocarcinoma |
| 28 | 18 | 5 | Kidney | Renal cell carcinoma |
| 46 | 17 | 8 | Rectum | Adenocarcinoma |
| 18 | 17 | 3 | Ovary | Endometrioid adenocarcinoma |
| 6 | 17 | 1 | Pleura | Mesothelioma, malignant |
| 18 | 17 | 3 | Soft tissues | Fibrous histiocytoma, malignant |
| 7 | 14 | 1 | Glial cell | Malignant glioma |
| 79 | 14 | 11 | Endometrium | Endometrioid adenocarcinoma |
| 15 | 13 | 2 | Epithelial cell | Carcinoma |
| 16 | 13 | 2 | Cecum | Adenocarcinoma |
| 38 | 11 | 4 | Ovary | Papillary serous adenocarcinoma |
| 19 | 11 | 2 | Thyroid gland | Papillary carcinoma |
| 10 | 10 | 1 | Descending | Adenocarcinoma |
| | | | colon | |
| 10 | 10 | 1 | Kidney | Wilms' tumor |
| 10 | 10 | 1 | Soft tissues | Sarcoma |
| 11 | 9 | 1 | Epithelial cell | Large cell carcinoma |
| 12 | 8 | 1 | Axillary lymph | Infiltrating duct carcinoma |
| | | | node | |
| 36 | 8 | 3 | Omentum | Papillary serous adenocarcinoma |
| 12 | 8 | 1 | Ovary | Adenocarcinoma |
| 12 | 8 | 1 | Stomach | Signet ring cell carcinoma |
| 13 | 8 | 1 | Ovary | Serous cystadenocarcinoma |
| 35 | 6 | 2 | White blood | Chronic lymphocytic leukemia |
| | | | cell | |
| 22 | 5 | 1 | Glial cell | Glioblastoma multiforme |
| 33 | 3 | 1 | Histiocyte | Histiocytic sarcoma |
| 80 | 3 | 2 | Lymphoblast | Burkitt's lymphoma |

TABLE 8
Expression of FGFR4 in Human Tumors

| | | Tumors | | |
|---|---|---|---|---|
| Tumors | | Expressing | | |
| Searched | % FGFR4 | FGFR4 | Sample Site | Pathology/Morphology |
| 1 | 100 | 1 | Abdominal lymph | Cholangiocarcinoma |
| | | | node | |
| 1 | 100 | 1 | Abdominal lymph | Mucinous adenocarcinoma |
| | | | node | |
| 1 | 100 | 1 | Axillary lymph node | Peripheral T-cell lymphoma |
| 1 | 100 | 1 | Cardia of stomach | Adenocarcinoma |
| 1 | 100 | 1 | Cecum | Hepatocellular carcinoma |
| 1 | 100 | 1 | Cell | Mullerian mixed tumor |
| 1 | 100 | 1 | Cell | Rhabdomyosarcoma |
| 1 | 100 | 1 | Colon | Dedifferentiated liposarcoma |
| 1 | 100 | 1 | Duodenum | Mucinous adenocarcinoma |
| 1 | 100 | 1 | Duodenum | Neuroendocrine carcinoma |
| 1 | 100 | 1 | Epithelial cell | Clear cell adenocarcinoma |
| 1 | 100 | 1 | Exocervix | Adenocarcinoma |
| 1 | 100 | 1 | Heart | Fibromyxosarcoma |
| 1 | 100 | 1 | Liver | Angiomyosarcoma |
| 1 | 100 | 1 | Liver | Fibrous histiocytoma, malignant |
| 3 | 100 | 3 | Liver | Hepatoblastoma |
| 1 | 100 | 1 | Liver | Islet cell carcinoma |
| 1 | 100 | 1 | Lung | Osteosarcoma |
| 1 | 100 | 1 | Ovary | Infiltrating duct and lobular |
| | | | | carcinoma |
| 1 | 100 | 1 | Ovary | Infiltrating lobular carcinoma |
| 1 | 100 | 1 | Pancreas | Acinar cell carcinoma |
| 1 | 100 | 1 | Parotid gland | Adenocarcinoma |
| 1 | 100 | 1 | Peritoneum | Sarcoma |
| 1 | 100 | 1 | Prostate | Rhabdomyosarcoma |
| 1 | 100 | 1 | Soft tissues | Clear cell adenocarcinoma |
| 2 | 100 | 2 | Soft tissues | Desmoplastic small round cell |
| | | | | tumor |
| 1 | 100 | 1 | Soft tissues | Primitive neuroectodermal tumor |
| 1 | 100 | 1 | Spleen | Hepatocellular carcinoma |
| 1 | 100 | 1 | Testis | Embryonal carcinoma |
| 22 | 77 | 17 | Liver | Hepatocellular carcinoma |
| 46 | 76 | 35 | Rectum | Adenocarcinoma; group as |
| | | | | colorectal cancer |
| 31 | 68 | 21 | Sigmoid colon | Adenocarcinoma; group as |
| | | | | colorectal cancer |
| 3 | 67 | 2 | Adrenal cortex | Adrenal cortical carcinoma |
| 18 | 67 | 12 | Esophagus | Adenocarcinoma |
| 27 | 67 | 18 | Liver | Adenocarcinoma |
| 3 | 67 | 2 | Liver | Cholangiocarcinoma |
| 2 | 50 | 1 | Breast | Papillary adenocarcinoma |
| 10 | 50 | 5 | Descending colon | Adenocarcinoma; group as |
| | | | | colorectal cancer |
| 2 | 50 | 1 | Endometrium | Clear cell adenocarcinoma |
| 4 | 50 | 2 | Gastroesophageal | Adenocarcinoma |
| | | | junction | |
| 2 | 50 | 1 | Lung | Branchiolo-alveolar |
| | | | | adenocarcinoma |
| 2 | 50 | 1 | Lung | Clear cell adenocarcinoma |
| 2 | 50 | 1 | Omentum | Infiltrating lobular carcinoma |
| 2 | 50 | 1 | Ovary | Dysgerminoma |
| 2 | 50 | 1 | Sigmoid colon | Mucinous adenocarcinoma |
| 10 | 50 | 5 | Testis | Mixed germ cell tumor |
| 35 | 49 | 17 | Ascending colon | Adenocarcinoma; group as |
| | | | | colorectal cancer |
| 26 | 46 | 12 | Colon | Adenocarcinoma; group as |
| | | | | colorectal cancer |
| 11 | 45 | 5 | Transverse colon | Adenocarcinoma; group as |
| | | | | colorectal cancer |
| 42 | 43 | 18 | Stomach | Adenocarcinoma |
| 5 | 40 | 2 | Ampulla of Vater | Adenocarcinoma |
| 10 | 40 | 4 | Pancreas | Islet cell carcinoma |
| 28 | 39 | 11 | Kidney | Renal cell carcinoma |
| 129 | 39 | 50 | Epithelial cell | Adenocarcinoma |
| 16 | 38 | 6 | Cecum | Adenocarcinoma |
| 8 | 38 | 3 | Ovary | Mucinous cystadenocarcinoma |
| 11 | 36 | 4 | Endometrium | Mullerian mixed tumor |
| 12 | 33 | 4 | Axillary lymph node | Infiltrating duct carcinoma |
| 3 | 33 | 1 | Brain | Adenocarcinoma |
| 3 | 33 | 1 | Colon | Mucinous adenocarcinoma |
| 9 | 33 | 3 | Epithelial cell | Infiltrating duct carcinoma |
| 12 | 33 | 4 | Ovary | Adenocarcinoma |
| 3 | 33 | 1 | Ovary | Mucinous adenocarcinoma |
| 10 | 30 | 3 | Ovary | Clear cell adenocarcinoma |
| 61 | 30 | 18 | Kidney | Clear cell adenocarcinoma |
| 4 | 25 | 1 | Breast | Mucinous adenocarcinoma |
| 4 | 25 | 1 | Endometrium | Papillary serous adenocarcinoma |
| 4 | 25 | 1 | Soft tissues | Liposarcoma |
| 9 | 22 | 2 | Duodenum | Adenocarcinoma |
| 9 | 22 | 2 | Soft tissues | Adenocarcinoma |
| 5 | 20 | 1 | Cecum | Mucinous adenocarcinoma |
| 5 | 20 | 1 | Omentum | Mullerian mixed tumor |
| 5 | 20 | 1 | Ovary | Mullerian mixed tumor |
| 5 | 20 | 1 | Stomach | Mucinous adenocarcinoma |
| 10 | 20 | 2 | Testis | Seminoma |
| 11 | 18 | 2 | Omentum | Adenocarcinoma |
| 6 | 17 | 1 | Ovary | Carcinoma |
| 91 | 13 | 12 | Lung | Adenocarcinoma |
| 31 | 13 | 4 | Breast | Infiltrating lobular carcinoma |
| 16 | 13 | 2 | Breast | Infiltrating duct and lobular |
| | | | | carcinoma |
| 57 | 12 | 7 | Pancreas | Adenocarcinoma |
| 279 | 11 | 30 | Breast | Infiltrating duct carcinoma |
| 10 | 10 | 1 | Kidney | Wilms' tumor |
| 10 | 10 | 1 | Skin | Malignant melanoma |
| 11 | 9 | 1 | Abdominal lymph | Adenocarcinoma |
| | | | node | |
| 11 | 9 | 1 | Epithelial cell | Large cell carcinoma |
| 12 | 8 | 1 | Stomach | Signet ring cell carcinoma |
| 13 | 8 | 1 | Lung | Carcinoma |
| 13 | 8 | 1 | Ovary | Serous cystadenocarcinoma |
| 15 | 7 | 1 | Epithelial cell | Carcinoma |
| 79 | 5 | 4 | Endometrium | Endometrioid adenocarcinoma |
| 72 | 1 | 1 | Lung | Squamous cell carcinoma |
| 80 | 1 | 1 | Lymphoblast | Burkitt's lymphoma |

TABLE 9
Detection of FGFR1-4 Gene Products by PCR

| Reverse Transcription Mixture | | |
|---|---|---|
| 10x Reverse Transcription Buffer | 10 | μl |
| 25x dNTPs | 4 | μl |
| 10x random primers | 10 | μl |
| MultiScribe Reverse Transcriptase, 50 U/μl | 5 | μl |
| Nuclease-free water | 16 | μl |
| Rnase inhibitor | 5 | μl |
| Total RNA (up to 10 μg RNA in H₂O) | 50 | μl |
| total | 100 | μl |
| Reverse Transcription reaction | | |
| 25° C. | 10 | min |
| 37° C. | 120 | min |
| PCR Mixture with Assay-On-Demand primers/probe set | | |
| 2x TaqMan Universal Master Mix | 12.5 | μl |
| 20x primer/probe set | 1.25 | μl |
| H₂O | 1.25 | μl |
| cDNA | 10 | μl |
| total | 50 | μl |
| PCR mixture with customer Primers/probe set | | |
| Final concentration: 900 nM for primers and 250 nM for | | |
| probe | | |
| 2x TaqMan Universal Master Mix | 12.5 | μl |
| Forward primer | 1 | μl |
| Reverse primer | 1 | μl |
| probe | 0.5 | μl |
| cDNA | 10 | μl |
| total | 25 | μl |
| PCR Reaction | | |
| 50° C. | 2 | min |
| 95° C. | 10 | min |
| 40 cycles of | | |
| 95° C. | 15 | sec |
| 60° C. | 1 | min |

While the present invention has been described with reference to the specific embodiments thereof, it should be understood by those skilled in the art that various changes may be made and equivalents may be substituted without departing from the true spirit and scope of the invention. In addition, many modifications can be made to adapt a particular situation, material, composition of matter, process, process step or steps, to the objective, spirit and scope of the present invention. All such modifications are intended to be within the scope of the claims appended hereto.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims. Moreover, advantages described in the body of the specification, if not included in the claims, are not per se limitations to the claimed invention.

It is to be understood that both the foregoing general description and the following detailed description are exemplary and explanatory only and are not restrictive of the invention, as claimed. Moreover, it must be understood that the invention is not limited to the particular embodiments described, as such may, of course, vary. Further, the terminology used to describe particular embodiments is not intended to be limiting, since the scope of the present invention will be limited only by its claims.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention. Further, all publications mentioned herein are incorporated by reference.

It must be noted that, as used herein and in the appended claims, the singular forms “a,” “or,” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### EXAMPLES

The examples, which are intended to be purely exemplary of the invention and should therefore not be considered to limit the invention in any way, also describe and detail aspects and embodiments of the invention discussed above. The examples are not intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Assay for FGFR Antibody Function

A cell line that does not express FGFR, e.g., L6 cells, will be individually stably transfected with each of the different FGFR constructs shown herein. FGFR antibodies will be tested for agonist or antagonist activity in proliferation assays, or another suitable assays, e.g., phospho-ERK or phospho-AKT assays, as described by Beer et al., J Biol Clem. 275(21):16091 (2000). To test for antagonist antibodies, cells expressing an FGFR will be pre-treated with the putative blocking antibody before stimulating the cells with FGF. FGF1 (acidic FGF) and/or FGF2 (basic FGF) will be used to activate most or all FGFRs. In other assays, more selective FGFs will be used as appropriate, i.e., FGF7 (KGF) for the FGFR2 IIIb. Control experiments will be performed as above using pre-immune serum instead of the putative blocking antibodies. To test for agonist antibodies, cells will be stimulated with the putative agonist antibodies, and proliferation, phospho-ERK or phospho-AKT activity will be measured according to known protocols.

Proliferation assays will be performed by serum-starving cells for 24 hours before the addition of the antibodies and FGFs, and proliferation measured over an appropriate time course. Proliferation will be determined by measuring ATP levels with the Cell Titer Glo (Promega) system. Phospho-AKT and Phospho-ERK will be measured in ELISA-based assays, following the manufacturer's instructions (Cell Signaling).

### Example 2

### Detection of FGFR1-4 Gene Products by PCR

The subject polynucleotide compositions can be used as probes and primers in hybridization applications, e.g., polymerase chain reaction (PCR); the identification of expression patterns in biological specimens; the preparation of cell or animal models for function of the subject polypeptide; the preparation of in vitro models for function of the subject polypeptide; etc.

Human genomic polynucleotide sequences corresponding to the cDNA polynucleotide sequences of the present invention as among sequences comprising the genes of or encoding any of SEQ ID NOs. 1-80 or variants thereof, may be identified by any conventional means, such as, for example, by probing a genomic DNA library with all or a portion of the present polynucleotide sequences.

Small DNA fragments are useful as primers for PCR, hybridization screening probes, etc. Larger DNA fragments, i.e., greater than 100 nt are useful for production of the encoded polypeptide. For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other.

The DNA may also be used to identify expression of the gene in a biological specimen. The manner in which one probes cells for the presence of particular nucleotide sequences, as genomic DNA or RNA, is well established in the literature. Briefly, DNA or mRNA is isolated from a cell sample. The mRNA may be amplified by RT-PCR, using reverse transcriptase to form a complementary DNA strand, followed by polymerase chain reaction amplification using primers specific for the subject DNA sequences. Alternatively, the mRNA sample is separated by gel electrophoresis, transferred to a suitable support, e.g., nitrocellulose, nylon, etc., and then probed with a fragment of the subject DNA as a probe. Other techniques, such as oligonucleotide ligation assays, in situ hybridizations, and hybridization to DNA probes arrayed on a solid chip may also find use. Detection of mRNA hybridizing to the subject sequence is indicative of gene expression in the sample.

To design the forward primer for PCR amplification, the melting point of the first 20 to 24 bases of the primer can be calculated by counting total A and T residues, then multiplying by 2. To design the reverse primer for PCR amplification, the melting point of the first 20 to 24 bases of the reverse complement, with the sequences written from 5-prime to 3-prime can be calculated by counting the total G and C residues, then multiplying by 4. Both start and stop codons can be present in the final amplified clone. The length of the primers is such to obtain melting temperatures within 59 degrees C. to 70 degrees C.

Full length PCR can be achieved by creating a reaction composition comprising, primers diluted to 5 μM in water, into a reaction vessel and adding a reaction mixture composed of 1× Taq buffer, 25 mM dNTP, 10 ng cDNA pool or genomic DNA, TaqPlus (Stratagene, Calif.) (5 u/ul), PfuTurbo (Stratagene, Calif.) (2.5 u/ul), and water. The contents of the reaction vessel are then mixed gently by inversion 5-6 times, placed into a reservoir where 2 μl F₁/R₁ primers are added, the plate sealed and placed in the thermocycler. The PCR reaction is comprised of the following eight steps. Step 1: 95° C. for 3 min. Step 2: 94° C. for 45 sec. Step 3: 0.50° C./sec to 56-60° C. Step 4: 56-60° C. for 50 sec. Step 5: 72° C. for 5 min. Step 6: Go to step 2, perform 35-40 cycles. Step 7: 72° C. for 20 min. Step 8: 4° C.

In one embodiment of the invention, gene expression of FGFR3 IIIb and IIIc isoforms were monitored using the TaqMan system (Applied Biosystems, CA). TaqMan comprises a method of real time PCR measurements, wherein the method used a thermocycler, a laser to induce fluorescence, CCD (charge-coupled device) detector, real-time sequence detection software, and TaqMan reagents. The cycle-by-cycle detection of the increase in the amount of PCR product was quantified in real time by using special probes, wherein a “reporter dye” attached to the 5′ end of the TaqMan probe, fluoresced when it was separated from the “quencher” linked to the 3′ end of the probe during the PCR extension cycle.

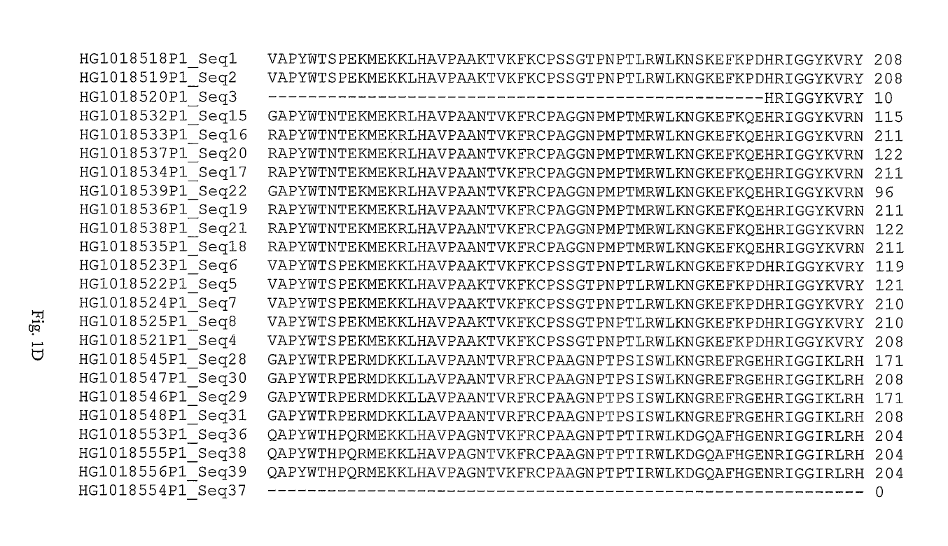

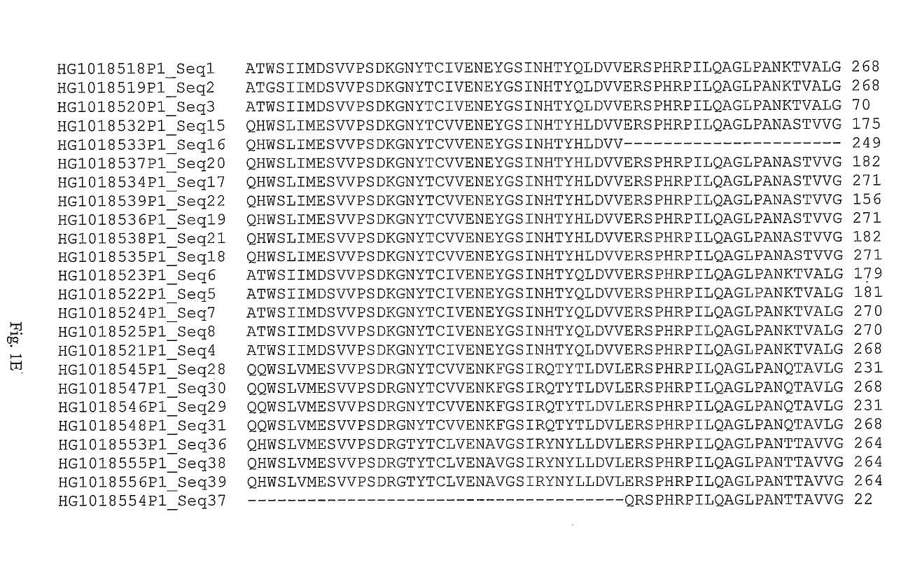

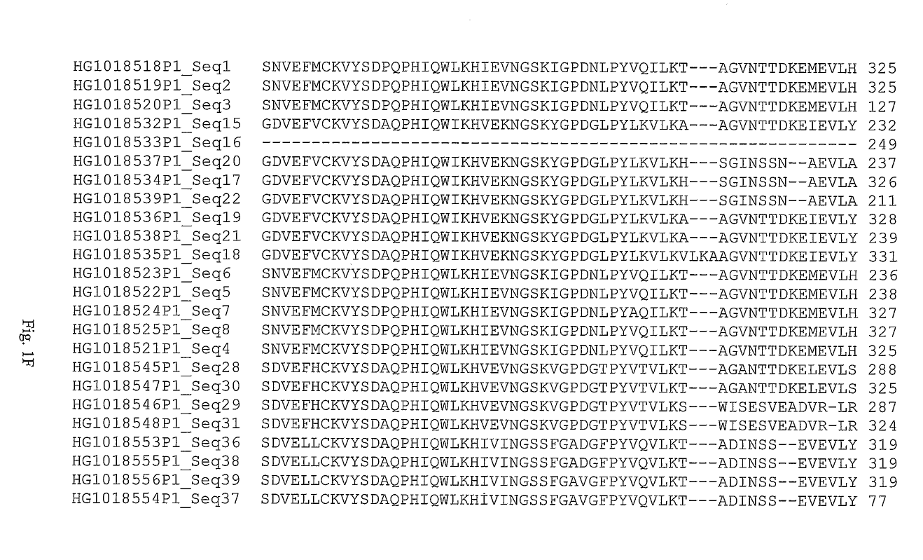

The materials used for the TaqMan real-time PCR of human FGFR3 included total RNA isolated internally from different tissues; High-Capacity cDNA Archive I(it for reverse transcription (Applied Biosystems, CA); RNase inhibitor (Applied Biosystems, CA); Taqman Universal PCR Master Mix (Applied Biosystems, CA); primers and probe for eukaryotic 18 S ribosomal RNA as the internal control (Assay-On-Demand primers/probe set) (Applied Biosystems, CA). The specific primers and probes for FGFR3 IIIb and FGFR3 IIIc, respectively, were designed internally and were synthesized commercially (Applied Biosystems, CA). For FGFR3 IIIb, the forward primer was TGCTCAAGTCCTGGATCAGTGA (SEQ ID NO. 81); the reverse primer was GTGAACGCTCAGCCAAAAGG (SEQ ID NO. 82); and the probe was 6-FAM labeled TGTGTCGGAGCGGGA (SEQ ID NO. 83). For FGFR3 IIIc, the forward primer was ACAAGGAGCTAGAGGTTCTCTCCTT (SEQ ID NO. 84); the reverse primer was GCAGAGTGATGAGAAAACCCAATAG (SEQ ID NO. 85); and the probe was 6-FAM labeled CACCTTTGAGGACGCCG (SEQ ID NO. 86). The protocols used for the reverse transcription and PCR procedures are described in Table 8. Internal expression of FGFR3 IIIb and FGFR3 IIIc was controlled by observing both the expression of 18 S rRNA and glyceraldehyde phosphate dehydrogenase (GAPDH), as shown in FIG. 2.

The relative expression of each gene is indicated as ½^{Ct}, where Ct is the threshold cycle. The normalized relative gene expression is the relative expression of each tested gene (FGFR3 IIIb or FGFR3 IIIc) divided by the relative expression of 18 S RNA of the same tissue sample, which is 2^{Ct(18S RNA)/}2^{Ct(tested gene)}. Each bar represents the normalized relative tested gene expression in one tissue sample, with shaded bars for FGFR3 IIIb and white bars for FGFR3 IIIc. In total, there are 3 normal breast samples, 19 malignant breast samples, 3 normal heart samples, 3 normal kidney samples, 2 normal liver samples, and 1 normal lung sample.

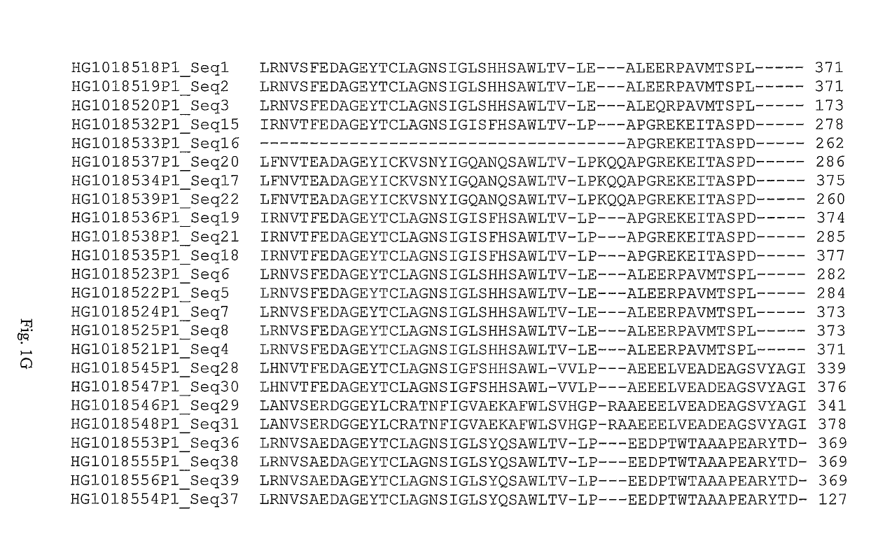

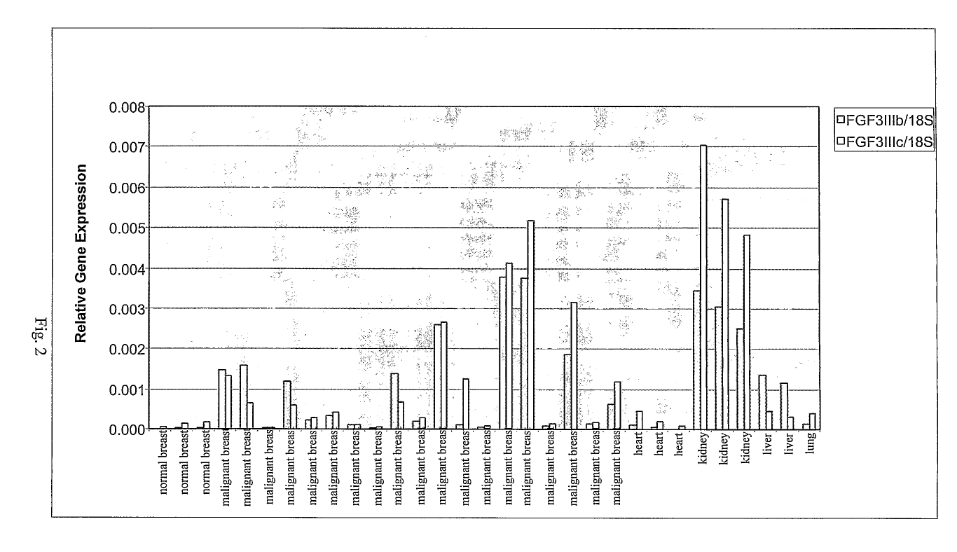

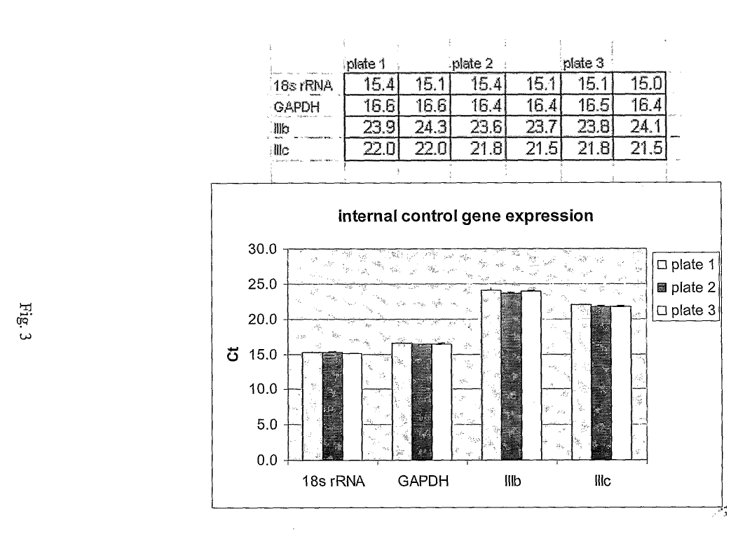

As seen in FIG. 3, among breast tissue samples, there is relatively low expression of FGFR3 IIIb as well as FGFR3 IIIc in normal breast tissues; there were about 50% malignant breast tissues (10 out of 19) having higher gene expression of both FGFR3 IIIb and FGFR3 IIIc as compared with normal breast tissues.

Among other normal tissues, both FGFR3 IIIb and FGFR3 IIIc were expressed at low level in normal heart and normal lung; FGFR3 IIIc was also expressed at low level in normal liver. However, FGFR3 IIIb was expressed at an intermediate level in normal liver; and both FGFR3 IIIb and FGFR3 IIIc were expressed at high level in normal kidney, equivalent or even higher compared with that in malignant breast tissue.

### Example 3

### Expression of FGFR in Human Tumors by Probing a GeneLogic Library Chip

The present inventors also interrogated a proprietary oncology database from GeneLogic, using Affymetrix U133 clip probe IDs that corresponded to certain of the sequences studied herein to determine the expression of the sequences in normal tissues and in cancer tissues.

Interrogation of the GeneLogic database showed overexpression of the FGFR3 gene in malignant bladder, ovary, breast, and endometrium, as compared to expression in the corresponding normal tissues. Furthermore, the database also showed high expression of FGFR3 in normal kidney, liver, lung, pancreas, and bladder. FGFR3, thus, is a strong target for production of therapeutic antibodies for treatment of tumors in which this gene is overexpressed or highly expressed, while minimizing the negative effects on the kidneys, liver, lung, pancreas, and bladder, where the gene is highly expressed and likely able to tolerate reductions to FGFR3 function.

Further interrogation of the GeneLogic database showed overexpression of the FGFR4 gene in malignant breast, endometrium, pancreas, rectum, and stomach, as compared to expression in the corresponding normal tissues. Furthermore, the database also showed high expression of FGFR3 in normal kidney, liver, lung, and colon. FGFR4, thus, is a strong target for production of therapeutic antibodies for treatment of tumors in which this gene is over or highly expressed, while minimizing the negative effects on the kidneys, liver, lung, and colon, where the gene is highly expressed and likely able to tolerate reductions to FGFR4 function.

### Example 4

### Expression of FGFR in Human Tumors by Computer Analysis of GeneLogic Database

A proprietary GeneLogic database was accessed to investigate whether FGFR1, FGFR2, FGFR3, and/or FGFR4 were expressed in a variety of malignant tumors. Table 5 shows the results for tumors expressing FGFR1. Table 6 shows the results for tumors expressing FGFR2. Table 7 shows the results for tumors expressing FGFR3. Finally, Table 8 shows the results for tumors expressing FGFR4. Briefly, all malignant tumors and their respective tumor sites found in the GeneLogic database were searched for expression of FGFR1, FGFR2, FGFR3, and/or FGFR4. The results were presented as number of tumors searched, percentage of those tumors searched that expressed the particular FGFR species, total number of tumors expressing the particular FGFR species, tumor site, and type of tumor pathology or morphology. The results of this inquiry can be used to provide useful therapeutic targets for the present invention. Antibodies of the invention that are specific to fragments of or theentirety of each of the FGFR species and can be applied as a therapeutic agent to those tumor types that express a particular FGFR.

### Example 5

### Detection of FGFR1-4 in Cells by Immunohistochemistry

The presence of the FGFR1, FGFR2, FGFR3 or FGFR4 proteins can be detected on cells using immunohistochemistry on frozen sections. Briefly, slides are fixed in acetone 15 min. at 4° C., then washed with PBS. Slides are next place in 3% H₂O₂ in PBS solution for 15 min., followed by PBS washing. 5% normal goat serum [Vector, Burlingame Calif.] is added to the slides and the slide is incubated at room temperature for 15 min, followed by one wash in PBS. The slides are next incubated with 5% skim milk (in PBS) for 15 min. and then washed three times in PBS. The slide is incubated with the primary antibody at 4° C. overnight, followed by washing the slide three times in PBS. If the primary antibody is a monoclonal antibody, a secondary antibody of biotinylated goat anti-mouse IgG (1:400) [DAKO E0433 Carpinteria Calif.] is added to the slide and incubated at room temperature for 30 min., followed by washing the slide three times in PBS. Alternatively, other biotinylated secondary antibodies can be used if the antibody is not a monoclonal, with such secondary antibodies being well-known in the art. The slide is then incubated with peroxidase-conjugated Avidin: [DAKO P0364 Carpinteria Calif.] (1:800) and incubated at room temperature for 30 min. The antigen-antibody reaction is demonstrated by using fresh DAB [DAKO K3466 Carpinteria Calif.] as substrate then counterstained with hematoxylin. Negative controls are performed by using the same concentration mouse IgG [DAKO K0931 Carpinteria Calif.]. Using this method, immunohistochemistry revealed the presence of FGFR3 in normal kidney tissue.

What is claimed is:

1. An isolated antibody that specifically binds to or interferes with the binding of one or more cell surface FGFRs or active fragments thereof, wherein the antibody is an agonist antibody and/or an antagonist antibody, wherein the antagonist antibody interferes with the function of one or more cell surface FGFRs, and wherein the agonist antibody activates one or more cell surface FGFRs.

2. The antibody of claim 1, wherein the cell surface protein is FGFR1 and the fragment comprises an amino acid sequence selected from SEQ ID NOs: 1-14, 43-45, and 55-60.

3. The antibody of claim 1, wherein the cell surface protein is FGFR2 and the fragment comprises an amino acid sequence selected from SEQ ID NOs: 15-27, 46-48, and 61-68.

4. The antibody of claim 1, wherein the cell surface protein is FGFR3 and the fragment comprises an amino acid sequence selected from SEQ ID NOs: 28-35, 49-51, and 69-75.

5. The antibody of claim 1, wherein the cell surface protein is FGFR4 and the fragment comprises an amino acid sequence selected from SEQ ID NOs: 36-42, 52-54, and 76-80.

6. The antibody of claim 1, wherein the antibody is an agonist antibody.

7. The antibody of claim 1, wherein the antibody is an antagonist antibody.

8. The antibody of claim 1, wherein the antibody does not induce antibody dependent cellular cytotoxicity.

9. The antibody of claim 1, wherein the antibody induces antibody dependent cellular cytotoxicity.

10. The antibody of claim 1, wherein the epitope consists of the amino acid sequence of SEQ ID NO. 1.

11. The antibody of claim 1, wherein the epitope consists of the amino acid sequence of SEQ ID NO. 2.

12. The antibody of claim 1, wherein the epitope consists of the amino acid sequence of SEQ ID NO. 3.

13. The antibody of claim 1, wherein the epitope consists of the amino acid sequence of SEQ ID NO. 4.

14. The antibody of claim 1, wherein the epitope consists of the amino acid sequence of SEQ ID NO. 5.

15. The antibody of claim 1, wherein the epitope consists of the amino acid sequence of SEQ ID NO. 6.

16. The antibody of claim 1, wherein the epitope consists of the amino acid sequence of SEQ ID NO. 7.

17. The antibody of claim 1, wherein the epitope consists of the amino acid sequence of SEQ ID NO. 8.

18. The antibody of claim 1, wherein the epitope consists of the amino acid sequence of SEQ ID NO. 9.

19. The antibody of claim 1, wherein the epitope consists of the amino acid sequence of SEQ ID NO. 10.

20. The antibody of claim 1, wherein the epitope consists of the amino acid sequence of SEQ ID NO. 11.

21. The antibody of claim 1, wherein the epitope consists of the amino acid sequence of SEQ ID NO. 12.

22. The antibody of claim 1, wherein the epitope consists of the amino acid sequence of SEQ ID NO. 13.

23. The antibody of claim 1, wherein the epitope consists of the amino acid sequence of SEQ ID NO. 14.

24. The antibody of claim 1, wherein the epitope consists of the amino acid sequence of SEQ ID NO. 15.

25. The antibody of claim 1, wherein the epitope consists of the amino acid sequence of SEQ ID NO. 16.

26. The antibody of claim 1, wherein the epitope consists of the amino acid sequence of SEQ ID NO. 17.

27. The antibody of claim 1, wherein the epitope consists of the amino acid sequence of SEQ ID NO. 18.

28. The antibody of claim 1, wherein the epitope consists of the amino acid sequence of SEQ ID NO. 19.

29. The antibody of claim 1, wherein the epitope consists of the amino acid sequence of SEQ ID NO. 20.

30. The antibody of claim 1, wherein the epitope consists of the amino acid sequence of SEQ ID NO. 21.

31. The antibody of claim 1, wherein the epitope consists of the amino acid sequence of SEQ ID NO. 22.

32. The antibody of claim 1, wherein the epitope consists of the amino acid sequence of SEQ ID NO. 23.

33. The antibody of claim 1, wherein the epitope consists of the amino acid sequence of SEQ ID NO. 24.

34. The antibody of claim 1, wherein the epitope consists of the amino acid sequence of SEQ ID NO. 25.

35. The antibody of claim 1, wherein the epitope consists of the amino acid sequence of SEQ ID NO. 26.

36. The antibody of claim 1, wherein the epitope consists of the amino acid sequence of SEQ ID NO. 27.

37. The antibody of claim 1, wherein the epitope consists of the amino acid sequence of SEQ ID NO. 28.

38. The antibody of claim 1, wherein the epitope consists of the amino acid sequence of SEQ ID NO. 29.

39. The antibody of claim 1, wherein the epitope consists of the amino acid sequence of SEQ ID NO. 30.

40. The antibody of claim 1, wherein the epitope consists of the amino acid sequence of SEQ ID NO. 31.

41. The antibody of claim 1, wherein the epitope consists of the amino acid sequence of SEQ ID NO. 32.

42. The antibody of claim 1, wherein the epitope consists of the amino acid sequence of SEQ ID NO. 33.

43. The antibody of claim 1, wherein the epitope consists of the amino acid sequence of SEQ ID NO. 34.

44. The antibody of claim 1, wherein the epitope consists of the amino acid sequence of SEQ ID NO. 35.

45. The antibody of claim 1, wherein the epitope consists of the amino acid sequence of SEQ ID NO. 36

46. The antibody of claim 1, wherein the epitope consists of the amino acid sequence of SEQ ID NO. 37.

47. The antibody of claim 1, wherein the epitope consists of the amino acid sequence of SEQ ID NO. 38.

48. The antibody of claim 1, wherein the epitope consists of the amino acid sequence of SEQ ID NO. 39.

49. The antibody of claim 1, wherein the epitope consists of the amino acid sequence of SEQ ID NO. 40.

50. The antibody of claim 1, wherein the epitope consists of the amino acid sequence of SEQ ID NO. 41.

51. The antibody of claim 1, wherein the epitope consists of the amino acid sequence of SEQ ID NO. 42.

52. The antibody of claim 1, wherein the epitope consists of the amino acid sequence of SEQ ID NO. 43.

53. The antibody of claim 1, wherein the epitope consists of the amino acid sequence of SEQ ID NO. 44.

54. The antibody of claim 1, wherein the epitope consists of the amino acid sequence of SEQ ID NO. 45.

55. The antibody of claim 1, wherein the epitope consists of the amino acid sequence of SEQ ID NO. 46.

56. The antibody of claim 1, wherein the epitope consists of the amino acid sequence of SEQ ID NO. 47.

57. The antibody of claim 1, wherein the epitope consists of the amino acid sequence of SEQ ID NO. 48.

58. The antibody of claim 1, wherein the epitope consists of the amino acid sequence of SEQ ID NO. 49.

59. The antibody of claim 1, wherein the epitope consists of the amino acid sequence of SEQ ID NO. 50.

60. The antibody of claim 1, wherein the epitope consists of the amino acid sequence of SEQ ID NO. 51.

61. The antibody of claim 1, wherein the epitope consists of the amino acid sequence of SEQ ID NO. 52.

62. The antibody of claim 1, wherein the epitope consists of the amino acid sequence of SEQ ID NO. 53.

63. The antibody of claim 1, wherein the epitope consists of the amino acid sequence of SEQ ID NO. 54.

64. The antibody of claim 1, wherein the epitope consists of the amino acid sequence of SEQ ID NO. 55.

65. The antibody of claim 1, wherein the epitope consists of the amino acid sequence of SEQ ID NO. 56.

66. The antibody of claim 1, wherein the epitope consists of the amino acid sequence of SEQ ID NO. 57.

67. The antibody of claim 1, wherein the epitope consists of the amino acid sequence of SEQ ID NO. 58.

68. The antibody of claim 1, wherein the epitope consists of the amino acid sequence of SEQ ID NO. 59.

69. The antibody of claim 1, wherein the epitope consists of the amino acid sequence of SEQ ID NO. 60.

70. The antibody of claim 1, wherein the epitope consists of the amino acid sequence of SEQ ID NO. 61.

71. The antibody of claim 1, wherein the epitope consists of the amino acid sequence of SEQ ID NO. 62.

72. The antibody of claim 1, wherein the epitope consists of the amino acid sequence of SEQ ID NO. 63.

73. The antibody of claim 1, wherein the epitope consists of the amino acid sequence of SEQ ID NO. 64.

74. The antibody of claim 1, wherein the epitope consists of the amino acid sequence of SEQ ID NO. 65.

75. The antibody of claim 1, wherein the epitope consists of the amino acid sequence of SEQ ID NO. 66

76. The antibody of claim 1, wherein the epitope consists of the amino acid sequence of SEQ ID NO. 67.

77. The antibody of claim 1, wherein the epitope consists of the amino acid sequence of SEQ ID NO. 68.

78. The antibody of claim 1, wherein the epitope consists of the amino acid sequence of SEQ ID NO. 69.

79. The antibody of claim 1, wherein the epitope consists of the amino acid sequence of SEQ ID NO. 70.

80. The antibody of claim 1, wherein the epitope consists of the amino acid sequence of SEQ ID NO. 71.

81. The antibody of claim 1, wherein the epitope consists of the amino acid sequence of SEQ ID NO. 72.

82. The antibody of claim 1, wherein the epitope consists of the amino acid sequence of SEQ ID NO. 73.

83. The antibody of claim 1, wherein the epitope consists of the amino acid sequence of SEQ ID NO. 74.

84. The antibody of claim 1, wherein the epitope consists of the amino acid sequence of SEQ ID NO. 75.

85. The antibody of claim 1, wherein the epitope consists of the amino acid sequence of SEQ ID NO. 76.

86. The antibody of claim 1, wherein the epitope consists of the amino acid sequence of SEQ ID NO. 77.

87. The antibody of claim 1, wherein the epitope consists of the amino acid sequence of SEQ ID NO. 78.

88. The antibody of claim 1, wherein the epitope consists of the amino acid sequence of SEQ ID NO. 79.

89. The antibody of claim 1, wherein the epitope consists of the amino acid sequence of SEQ ID NO. 80.

90. The antibody of claim 1, wherein the epitope consists essentially of the amino acid sequence of SEQ ID NO. 1.

91. The antibody of claim 1, wherein the epitope consists essentially of the amino acid sequence of SEQ ID NO. 2.

92. The antibody of claim 1, wherein the epitope consists essentially of the amino acid sequence of SEQ ID NO. 3.

93. The antibody of claim 1, wherein the epitope consists essentially of the amino acid sequence of SEQ ID NO. 4.

94. The antibody of claim 1, wherein the epitope consists essentially of the amino acid sequence of SEQ ID NO. 5.

95. The antibody of claim 1, wherein the epitope consists essentially of the amino acid sequence of SEQ ID NO. 6.

96. The antibody of claim 1, wherein the epitope consists essentially of the amino acid sequence of SEQ ID NO. 7.

97. The antibody of claim 1, wherein the epitope consists essentially of the amino acid sequence of SEQ ID NO. 8.

98. The antibody of claim 1, wherein the epitope consists essentially of the amino acid sequence of SEQ ID NO. 9.

99. The antibody of claim 1, wherein the epitope consists essentially of the amino acid sequence of SEQ ID NO. 10.

100. The antibody of claim 1, wherein the epitope consists essentially of the amino acid sequence of SEQ ID NO. 11.

101. The antibody of claim 1, wherein the epitope consists essentially of the amino acid sequence of SEQ ID NO. 12.

102. The antibody of claim 1, wherein the epitope consists essentially of the amino acid sequence of SEQ ID NO. 13.

103. The antibody of claim 1, wherein the epitope consists essentially of the amino acid sequence of SEQ ID NO. 14.

104. The antibody of claim 1, wherein the epitope consists essentially of the amino acid sequence of SEQ ID NO. 15.

105. The antibody of claim 1, wherein the epitope consists essentially of the amino acid sequence of SEQ ID NO. 16.

106. The antibody of claim 1, wherein the epitope consists essentially of the amino acid sequence of SEQ ID NO. 17.

107. The antibody of claim 1, wherein the epitope consists essentially of the amino acid sequence of SEQ ID NO. 18.

108. The antibody of claim 1, wherein the epitope consists essentially of the amino acid sequence of SEQ ID NO. 19.

109. The antibody of claim 1, wherein the epitope consists essentially of the amino acid sequence of SEQ ID NO. 20.

110. The antibody of claim 1, wherein the epitope consists essentially of the amino acid sequence of SEQ ID NO. 21.

111. The antibody of claim 1, wherein the epitope consists essentially of the amino acid sequence of SEQ ID NO. 22.

112. The antibody of claim 1, wherein the epitope consists essentially of the amino acid sequence of SEQ ID NO. 23.

113. The antibody of claim 1, wherein the epitope consists essentially of the amino acid sequence of SEQ ID NO. 24.

114. The antibody of claim 1, wherein the epitope consists essentially of the amino acid sequence of SEQ ID NO. 25.

115. The antibody of claim 1, wherein the epitope consists essentially of the amino acid sequence of SEQ ID NO. 26.

116. The antibody of claim 1, wherein the epitope consists essentially of the amino acid sequence of SEQ ID NO. 27.

117. The antibody of claim 1, wherein the epitope consists essentially of the amino acid sequence of SEQ ID NO. 28.

118. The antibody of claim 1, wherein the epitope consists essentially of the amino acid sequence of SEQ ID NO. 29.

119. The antibody of claim 1, wherein the epitope consists essentially of the amino acid sequence of SEQ ID NO. 30.

120. The antibody of claim 1, wherein the epitope consists essentially of the amino acid sequence of SEQ ID NO. 31.

121. The antibody of claim 1, wherein the epitope consists essentially of the amino acid sequence of SEQ ID NO. 32.

122. The antibody of claim 1, wherein the epitope consists essentially of the amino acid sequence of SEQ ID NO. 33.

123. The antibody of claim 1, wherein the epitope consists essentially of the amino acid sequence of SEQ ID NO. 34.

124. The antibody of claim 1, wherein the epitope consists essentially of the amino acid sequence of SEQ ID NO. 35.

125. The antibody of claim 1, wherein the epitope consists essentially of the amino acid sequence of SEQ ID NO. 36

126. The antibody of claim 1, wherein the epitope consists essentially of the amino acid sequence of SEQ ID NO. 37.

127. The antibody of claim 1, wherein the epitope consists essentially of the amino acid sequence of SEQ ID NO. 38.

128. The antibody of claim 1, wherein the epitope consists essentially of the amino acid sequence of SEQ ID NO. 39.

129. The antibody of claim 1, wherein the epitope consists essentially of the amino acid sequence of SEQ ID NO. 40.

130. The antibody of claim 1, wherein the epitope consists essentially of the amino acid sequence of SEQ ID NO. 41.

131. The antibody of claim 1, wherein the epitope consists essentially of the amino acid sequence of SEQ ID NO. 42.

132. The antibody of claim 1, wherein the epitope consists essentially of the amino acid sequence of SEQ ID NO. 43.

133. The antibody of claim 1, wherein the epitope consists essentially of the amino acid sequence of SEQ ID NO. 44.

134. The antibody of claim 1, wherein the epitope consists essentially of the amino acid sequence of SEQ ID NO. 45.

135. The antibody of claim 1, wherein the epitope consists essentially of the amino acid sequence of SEQ ID NO. 46.

136. The antibody of claim 1, wherein the epitope consists essentially of the amino acid sequence of SEQ ID NO. 47.

137. The antibody of claim 1, wherein the epitope consists essentially of the amino acid sequence of SEQ ID NO. 48.

138. The antibody of claim 1, wherein the epitope consists essentially of the amino acid sequence of SEQ ID NO. 49.

139. The antibody of claim 1, wherein the epitope consists essentially of the amino acid sequence of SEQ ID NO. 50.

140. The antibody of claim 1, wherein the epitope consists essentially of the amino acid sequence of SEQ ID NO. 51.

141. The antibody of claim 1, wherein the epitope consists essentially of the amino acid sequence of SEQ ID NO. 52.

142. The antibody of claim 1, wherein the epitope consists essentially of the amino acid sequence of SEQ ID NO. 53.

143. The antibody of claim 1, wherein the epitope consists essentially of the amino acid sequence of SEQ ID NO. 54.

144. The antibody of claim 1, wherein the epitope consists essentially of the amino acid sequence of SEQ ID NO. 55.

145. The antibody of claim 1, wherein the epitope consists essentially of the amino acid sequence of SEQ ID NO. 56.

146. The antibody of claim 1, wherein the epitope consists essentially of the amino acid sequence of SEQ ID NO. 57.

147. The antibody of claim 1, wherein the epitope consists essentially of the amino acid sequence of SEQ ED NO. 58.

148. The antibody of claim 1, wherein the epitope consists essentially of the amino acid sequence of SEQ ID NO. 59.

149. The antibody of claim 1, wherein the epitope consists essentially of the amino acid sequence of SEQ ID NO. 60.

150. The antibody of claim 1, wherein the epitope consists essentially of the amino acid sequence of SEQ ID NO. 61.

151. The antibody of claim 1, wherein the epitope consists essentially of the amino acid sequence of SEQ ID NO. 62.

152. The antibody of claim 1, wherein the epitope consists essentially of the amino acid sequence of SEQ ID NO. 63.

153. The antibody of claim 1, wherein the epitope consists essentially of the amino acid sequence of SEQ ID NO. 64.

154. The antibody of claim 1, wherein the epitope consists essentially of the amino acid sequence of SEQ ID NO. 65.

155. The antibody of claim 1, wherein the epitope consists essentially of the amino acid sequence of SEQ ID NO. 66

156. The antibody of claim 1, wherein the epitope consists essentially of the amino acid sequence of SEQ ID NO. 67.

157. The antibody of claim 1, wherein the epitope consists essentially of the amino acid sequence of SEQ ID NO. 68.

158. The antibody of claim 1, wherein the epitope consists essentially of the amino acid sequence of SEQ ID NO. 69.

159. The antibody of claim 1, wherein the epitope consists essentially of the amino acid sequence of SEQ ID NO. 70.

160. The antibody of claim 1, wherein the epitope consists essentially of the amino acid sequence of SEQ ID NO. 71.

161. The antibody of claim 1, wherein the epitope consists essentially of the amino acid sequence of SEQ ID NO. 72.

162. The antibody of claim 1, wherein the epitope consists essentially of the amino acid sequence of SEQ ID NO. 73.

163. The antibody of claim 1, wherein the epitope consists essentially of the amino acid sequence of SEQ ID NO. 74.

164. The antibody of claim 1, wherein the epitope consists essentially of the amino acid sequence of SEQ ID NO. 75.

165. The antibody of claim 1, wherein the epitope consists essentially of the amino acid sequence of SEQ ID NO. 76.

166. The antibody of claim 1, wherein the epitope consists essentially of the amino acid sequence of SEQ ID NO. 77.

167. The antibody of claim 1, wherein the epitope consists essentially of the amino acid sequence of SEQ ID NO. 78.

168. The antibody of claim 1, wherein the epitope consists essentially of the amino acid sequence of SEQ ID NO. 79.

169. The antibody of claim 1, wherein the epitope consists essentially of the amino acid sequence of SEQ ID NO. 80.

170. The antibody of claim 1, wherein the epitope of the cell surface FGFR is encoded by a polynucleotide sequence chosen from a polynucleotide that encodes a fragment of or the entirety of one or more of SEQ ID NOs. 1-80.

171. The antibody of claim 1, comprising at least one F_{ab} fragment of a first antibody linked to an F_{c} fragment of a second antibody, wherein the first and second antibodies specifically bind to different epitopes.

172. The antibody of claim 1, wherein the antibody is a polyclonal antibody.

173. The antibody of claim 1, wherein the antibody is a monoclonal antibody.

174. The antibody of claim 1, wherein the antibody is a single chain antibody.

175. The antibody of claim 1, wherein the antibody is selected from a human antibody, a humanized non-human animal antibody, a primatized antibody, and a chimeric antibody.

176. The antibody of claim 175, wherein the non-human animal antibody is a non-human primate antibody, a rabbit antibody, or a mouse antibody.

177. The antibody of claim 1, wherein the antibody is derived from a non-human animal genetically engineered to produce antibodies that comprise substantially human antibody sequences.

178. The antibody of claim 1, wherein the antibody comprises an active fragment chosen from an F_{ab} fragment, a variable region of a heavy chain, a variable region of a light chain, a cdr region, and a framework fragment.

179. The antibody of claim 1, wherein the antibody is not cytotoxic to at least one of kidney and liver cells.

180. The agonist antibody of claim 1, wherein the antibody stimulates cell growth, cell proliferation, and/or cell repair.

181. Al antibody composition comprising the antibody of claim 1 and a pharmaceutically acceptable carrier or excipient.

182. The antibody composition of claim 181, wherein the antibody is an antagonist antibody.

183. The antibody composition of claim 181, wherein the antibody is an agonist antibody.

184. A method of making the antibody of claim 1 comprising:
(a) immunizing an animal with an epitope of the cell surface FGFR;
(b) selecting a spleen cell that produces an antibody that specifically binds to or interferes with the function of the cell surface FGFR;
(c) producing a hybridoma that secretes the antibody; and
(d) culturing the hybridoma to reproduce the antibody.

185. A method of treating disease in a subject comprising:
(a) providing the antibody composition of claim 181; and
(b) administering a therapeutically effective amount of the antibody composition to the subject.

186. The method of claim 185, wherein the disease is a proliferative disease.

187. The method of claim 186, wherein the proliferative disease is cancer.

188. The method of claim 187, wherein the cancer is breast cancer.

189. The method of claim 185, wherein the disease is refractory to treatment with an anti-HER2 antibody.

190. The method of claim 185, wherein the disease is refractory to treatment with an anti-EGFR antibody.

191. The method of claim 185, wherein the disease is mucositis.

192. The method of claim 185, wherein the disease is an inflammatory disease.

193. The method of claim 192, wherein the inflammatory disease is selected from rheumatoid arthritis, osteoarthritis, psoriasis, inflammatory bowel disease, multiple sclerosis, systemic lupus erythematosis, myocardial infarction, stroke, and fulminant liver failure.

194. The method of claim 185, wherein the disease is a metabolic disorder.

195. The method of claim 194, wherein the metabolic disorder is type II diabetes, obesity, phosphatemia, or osteoporosis.

196. The method of claim 185, wherein the antibody is administered to the subject locally or systemically.

197. The method of claim 196, wherein the antibody is administered intravenously, intraarticular, intraperitoneally, subcutaneously, topically, or transdermally.

198. The method of claim 185, wherein the gene encoding the cell surface FGFR in the subject is amplified when compared to a subject without the disease.

199. A method of detecting the presence of an amplified gene encoding a cell surface FGFR in a subject comprising:
(a) providing a polynucleotide probe that hybridizes under stringent conditions to a nucleic acid molecule encoding the cell surface FGFR;
(b) providing a sample obtained from the subject;
(c) allowing the polynucleotide probe and the sample to interact under conditions that allow for specific hybridization; and
(d) determining whether specific hybridization has occurred.

200. The method of claim 199, wherein the polynucleotide probe is chosen from a polynucleotide that encodes a fragment of or the entirety of one or more of SEQ ID NOs. 1-80.

201. A method of detecting the presence of an amplified gene encoding a cell surface FGFR in a subject comprising:
(a) providing an antibody that specifically binds to the amplified gene;
(b) providing a sample obtained from the subject;
(c) allowing the antibody and the sample to interact under conditions that allow for specific binding; and
(d) determining whether specific binding has occurred.

202. The method of claim 201, wherein the polynucleotide probe is chosen from a polynucleotide that encodes a fragment of or the entirety of one or more of SEQ ID NOs. 1-80.

203. A method of detecting the presence of an amplified cell surface FGFR gene in a subject comprising:
(a) providing an antibody that specifically binds to the amplified gene;
(b) providing a sample obtained from the subject;
(c) allowing the antibody and the sample to interact under conditions that allow for specific binding; and
(d) determining whether specific binding has occurred.

204. The antibody of claim 1, wherein the fragment lacks its naturally occurring leader sequence.

205. The antibody of claim 204, wherein the leader sequence is MWSWKCLLFWAVLVTATLC.

206. The antibody of claim 204, wherein the leader sequence is MWSWKCLLFWAVLVTATLCTA.

207. The antibody of claim 204, wherein the leader sequence is MWSWKCLLFWAVLVTATLCTARP.

208. The antibody of claim 204, wherein the leader sequence is MVSWGRFICLVVVTMATLSLA.

209. The antibody of claim 204, wherein the leader sequence is MVSWGRFICLVVVTMATLSLARP.

210. The antibody of claim 204, wherein the leader sequence is MGAPACALALCVAVA.

211. The antibody of claim 204, wherein the leader sequence is MGAPACALALCVAVAIVA.

212. The antibody of claim 204, wherein the leader sequence is MGAPACALALCVAVAIVAGA.

213. The antibody of claim 204, wherein the leader sequence is MGAPACALALCVAVAIVAGASS.

214. The antibody of claim 204, wherein the leader sequence is MRLLLALLGILLS.

215. The antibody of claim 204, wherein the leader sequence is MRLLLALLGILLSVP.

216. The antibody of claim 204, wherein the leader sequence is MRLLLALLGILLSVPG.
